(19) United States
(12) Patent Application Publication
(10) Pub. No.: US 2024/0384244 A1
(43) Pub. Date: Nov. 21, 2024

(54) METHODS AND COMPOSITIONS FOR TREATING DNMT3A DEFICIENCY-ASSOCIATED DISEASES

(71) Applicant: Timothy Ley, St. Louis, MO (US)

(72) Inventor: Timothy Ley, St. Louis, MO (US)

(73) Assignee: Washington University, St. Louis, MO (US)

(21) Appl. No.: 18/620,632

(22) Filed: Mar. 28, 2024

Related U.S. Application Data
(60) Provisional application No. 63/492,529, filed on Mar. 28, 2023.

(51) Int. Cl. C12N 9/10; A61K 31/706; A61K 38/07; A61K 48/00; C12N 15/86

(52) U.S. Cl. CPC C12N 9/1007; A61K 31/706; A61K 38/07; A61K 48/0008; C12N 15/86; C12N 2740/10043; C12Y 201/01037

(57) ABSTRACT

Treatment and prevention of DNMT3A deficiency-associated diseases are provided. Compositions for treatment or prevention include at least one cDNA vector and/or at least one therapeutic agent capable of restoring DNMT3L activity, restoring DNMT3L expression, increasing DNMT3L activity, increasing DNMT3L expression, and/or increasing DNMT3A enzymatic activity. Methods for treatment or prevention include administering to a subject a composition including the at least one cDNA vector and/or at least one therapeutic agent. Methods of increasing DNMT3A activity in a subject having a DNMT3A mutation, methods of reversing a hypomethylation phenotype in bone marrow cells of a subject having a DNMT3A mutation, and methods of promoting cancer cell death in a subject having Acute Myeloid Leukemia (AML) are also provided.

### CROSS-REFERENCE TO RELATED APPLICATIONS

This application claims priority from U.S. Provisional Application Ser. No. 63/492,529 filed on 28 Mar. 2023, which is incorporated herein by reference in its entirety.

### STATEMENT REGARDING FEDERALLY SPONSORED RESEARCH OR DEVELOPMENT

This invention was made with government support under CA101937 and CA197561 awarded by the National Institutes of Health. The government has certain rights in the invention.

### FIELD OF THE DISCLOSURE

The present disclosure generally relates to methods and compositions for treating or preventing DNMT3A deficiency-associated diseases.

### BRIEF DESCRIPTION OF THE DISCLOSURE

Among the various aspects of the present disclosure are methods and compositions for treating DNMT3A deficiency-associated diseases.

In one aspect of the present disclosure, a method of increasing DNMT3A activity in a subject having a DNMT3A mutation is provided. The method comprises: reactivating expression of DNMT3L by administering a retroviral vector comprising DNMT3L cDNA to the subject.

In some embodiments, the DNMT3A mutation is selected from a loss-of function mutation and a dominant negative mutation; the DNMT3A mutation is selected from R882H and R878H; and/or the subject has a DNMT3A deficiency-associated disease selected from leukemia, acute myeloid leukemia (AML), a lymphoid malignancy, clonal hematopoiesis, and DNMT3A Overgrowth Syndrome. In some embodiments, the method further comprises administering a therapeutic agent selected from an histone deacetylase (HDAC) inhibitor and a hypomethylating agent; and/or the therapeutic agent is selected from Azacitidine, Romidepsin, and 5-azacytidine.

In another aspect of the present disclosure, a method of reversing a hypomethylation phenotype in bone marrow cells of a subject having a DNMT3A mutation is provided. The method comprises: increasing DNMT3A activity by administering a retroviral vector comprising one or cDNA vectors selected from DNMT3L, a combination of DNMT3A and DNMT3L, and a combination of DNMT3A and DNMT3B.

In some embodiments, the retroviral vector comprising the DNMT3L cDNA is administered directly to the bone marrow cells of the subject; the DNMT3A mutation is selected from a loss-of function mutation and a dominant negative mutation; the DNMT3A mutation is selected from R882H and R878H; and/or the subject has a DNMT3A deficiency-associated disease selected from leukemia, acute myeloid leukemia (AML), a lymphoid malignancy, clonal hematopoiesis, and DNMT3A Overgrowth Syndrome. In some embodiments, the method further comprises administering a therapeutic agent selected from an histone deacetylase (HDAC) inhibitor and a hypomethylating agent; and/or the therapeutic agent is selected from Azacitidine, Romidepsin, and 5-azacytidine.

In a further aspect of the present disclosure, a method of promoting cancer cell death in a subject having Acute Myeloid Leukemia (AML) is provided. The method comprises: increasing DNMT3A activity by administering a retroviral vector comprising one or cDNA vectors selected from DNMT3L, a combination of DNMT3A and DNMT3L, and a combination of DNMT3A and DNMT3B.

In some embodiments, the retroviral vector comprising the DNMT3L cDNA is administered directly to the bone marrow cells of the subject; the DNMT3A mutation is a loss-of function mutation; the DNMT3A mutation is a dominant negative mutation; and/or the DNMT3A mutation is selected from R882H and R878H. The method comprises: increasing DNMT3A activity by administering a retroviral vector comprising one or cDNA vectors selected from DNMT3L, a combination of DNMT3A and DNMT3L, and a combination of DNMT3A and DNMT3B.

Yet further aspects of the present disclosure provide for methods of treating or preventing a DNMT3A deficiency-associated disease in a subject in need thereof, comprising administering to the subject a therapeutic agent capable of restoring or increasing DNMT3L activity or expression or increasing DNMT3A enzymatic activity. In some embodiments, the DNMT3A-deficiency associated disease is leukemia, acute myeloid leukemia (AML), a lymphoid malignancy, clonal hematopoiesis, or DNMT3A Overgrowth Syndrome. In some embodiments, the therapeutic agent is a genetic vector comprising a gene encoding DNMT3L or variants thereof. In some embodiments, administering the genetic vector to the subject results in overexpression of a DNMT3L gene product (e.g., DNMT3L) or variants thereof. In some embodiments, the subject has a loss-of-function mutation (e.g., a dominant negative mutation) in the DNMT3A gene. In some embodiments, the mutation is R882H or R878H. In some embodiments, the therapeutic agent is an HDAC1 inhibitor. In some embodiments, the therapeutic agent is Azacitidine or Romidepsin. In some embodiments, administering the therapeutic agent results in remethylation of bone marrow cells; remethylation, depletion, or death of diseased (e.g., cancer) cells, or correction of abnormal myeloid skewing. In some embodiments, the therapeutic agent is specifically delivered to hematopoietic cells or diseased (e.g., cancer) cells. In some embodiments, the therapeutic agent modulates a regulatory element or trans-acting factor responsible for DNMT3L silencing. In some embodiments, the therapeutic agent is CRISPR/Cas9 to inactivate a negative regulatory element or CRISPR/Cas9a to activate DNMT3L to restore DNMT3L gene activity.

Other objects and features will be in part apparent and in part pointed out hereinafter.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is an exemplary embodiment of Western blot showing levels of DNMT3L protein or ACTIN in K562 cells treated with Romidepsin in accordance with the present disclosure.

FIG. 2(A-B) is an exemplary embodiment of floxing efficiency in Dnmt×Vav1-Cre bone marrow cells, and complete blood counts from mice in accordance with the present disclosure. FIG. 2A: Floxing efficiency for Dnmt deficient mice and R878H mice, deduced from WGBS data, or RNA-seq data from the bone marrow cells of each genotype. FIG. 2B: Peripheral blood cell counts from 6-8-week-old, 3a KO, 3b KO, or DKO mice, or R878H mice. ANOVA testing was used to compare the blood counts among mice from different genotypes. None of the values were significant different among genotypes.

FIG. 3(A-H) is an exemplary embodiment of DNA methylation phenotypes of Dnmt3a, Dnmt3b, or doubly deficient mouse bone marrow cells in accordance with the present disclosure. FIG. 3A: Heatmap showing the methylation values for 10,724 DMRs defined by comparing WGBS data from wild-type (WT, n=9) versus Dnmt3a^{flox/flox}×Vav1-Cre (3a KO, n=4) samples. Values for the same DMRs were plotted passively for Dnmt3b^{flox/flox}×Vav1-Cre (3b KO), Dnmt3a^{flox/flox}×Dnmt3b^{flox/flox}×Vav1-Cre (DKO), and Dnmt3a^{R878H/+}×Vav1-Cre (R878H) samples. FIG. 3B: Aggregate (mean) methylation values from the 10,724 DMRs identified in FIG. 3A for each genotype. FIG. 3C: Heatmap showing the methylation values of 2012 DMRs defined by comparing WGBS data from WT (n=9) versus 3b KO (n=3) samples. Values for the same DMRs were plotted passively for the 3a KO, 3b KO, DKO, and R878H samples. FIG. 3D: Aggregate (mean) methylation values from the 2012 DMRs identified in (C) for each genotype. FIG. 3E: Heatmap showing the methylation values of 23,411 DMRs defined by comparing WGBS data from WT (n=9) versus DKO (n=3) samples. Values for the same DMRs were plotted passively for the 3a KO, 3b KO, and R878H samples. FIG. 3F: Aggregate (mean) methylation values from the 23,411 DMRs identified in FIG. 3E for each genotype. FIG. 3G: Heatmap showing the methylation values of 4453 DMRs defined by comparing WGBS data from WT (n=9) versus R878H (n=6) samples. Values for the same DMRs were plotted passively for the 3a KO, 3b KO, and DKO samples. FIG. 3H: Aggregate (mean) methylation values from the 4453 DMRs identified in FIG. 3G for each genotype.

FIG. 4(A-G) is an exemplary embodiment of characterization of methylation and cellular phenotypes in DNA methyltransferase-deficient mouse bone marrow cells in accordance with the present disclosure. FIG. 4A: Heatmap showing the methylation values for individual samples of DMRs from combining all the DMRs (“Unified” DMRs) generated from the comparisons of WT versus 3a KO, WT versus 3b KO, WT versus DKO, and WT versus R878H samples. FIG. 4B: Density plot of mean methylation values from the unified DMRs for 3a KO, 3b KO, DKO, and R878H samples. FIG. 4C: Mean CpG methylation values from WGBS of bone marrow cells from WT, 3a KO, 3b KO, DKO, and R878H samples. Mean values across all CpGs in annotated regions of the genome are shown. FIG. 4D: IGV view of a representative region of the Hoxa gene cluster. Mean methylation values for each CpG are shown as a bar ranging from 0 to 100% methylated for each genotype. Blue bars at the bottom indicate DMRs identified in this region. FIG. 4E: t-distributed Stochastic Neighbor Embedding (t-SNE) projections of merged scRNA-seq data from whole-bone marrow cells derived from WT (n=3, two males and one female), 3a KO, 3b KO, and DKO mice, 6 to 8 weeks of age (n=2 for each for each knockout genotype; all samples were from males) Cell populations were assigned according to the Haemopedia algorithm and manual review. B progenitors include pro-B cells and pre-B cells; DC, dendritic cell; GMP, granulocyte-monocyte progenitors; MDP, monocyte dendritic cell progenitors; MEP, megakaryocyte erythrocyte progenitor; MPP, multipotent progenitor; NK, natural killer cells. FIG. 4F: Progenitor population distributions for each genotype using the scRNA-seq data in FIG. 4E. *P<0.05 and **P<0.01 via analysis of variance (ANOVA) test. FIG. 4G: Mature cell population distributions for each genotype, using the scRNA-seq data in FIG. 4E. PMN, polymorphonuclear leukocyte, including neutrophils and basophils in FIG. 4E; Macro, macrophages; Mono, monocytes.

FIG. 5(A-D) is an exemplary embodiment of characterization of phenotypes in DNA methyltransferase deficient mouse bone marrow cells in accordance with the present disclosure. FIG. 5A: Density plots of mean methylation values for all CpGs for 3a KO, 3b KO, DKO, and R878H mice. FIG. 5B: Density plot of mean methylation values from the non-DMRs CpGs from the same samples as in (FIG. 5A). FIG. 5C: IGV view of a representative region of the Hoxa gene cluster. Each row represents a sample from a unique mouse. Methylation values for each CpG are shown as a bar ranging from 0-100% methylated in individual samples. Blue bars at the bottom indicate DMRs identified in this region. The red rectangle highlights the methylation phenotypes in different genotypes. FIG. 5D: t-SNE projections of merged scRNA-seq data from whole bone marrow cells derived from each genotype (WT, 3a KO, 3b KO, and DKO, 6-8 weeks of age) (n=2 each). Cell populations were assigned according to the Haemopedia algorithm, and manual review. B progenitors include Pro-B cells and Pre-B cells; DC, dendritic cell; GMP, granulocytemonocyte progenitors; MDP, monocyte dendritic cell progenitor; MEP, megakaryocyte erythrocyte progenitor; MPP, multipotent progenitor; NK, natural killer cells.

FIG. 6(A-D) is an exemplary embodiment of isoforms of Dnmt3a and Dnmt3b mRNA in mouse bone marrow samples from unmanipulated WT vs R878H samples in accordance with the present disclosure. FIG. 6A: Comparisons of the structure and homology of the major isoforms of DNMT3A, DNMT3B, and DNMT3L in humans and mice. FIG. 6B: Bulk RNA-seq data was obtained from multiple unique mice with each genotype. Each bar represents data from one mouse. Dnmt3a1 is the dominant isoform in both WT (n=10) vs. R878H (n=12) bone marrow samples. Dnmt3a1 represents 88.6% and 88.5% Dnmt3a transcripts in WT and R878H, respectively. FIG. 6C: Dnmt3b3 is the dominant isoform in both WT and R878H bone marrow samples. The differences between isoform use between the WT and R878H marrow samples were not significant for either gene. Dnmt3b3 represents 78.2% and 78.1% Dnmt3b transcript in WT and R878H, respectively. FIG. 6D: Expression of total Dnmt3a vs. Dnmt3b mRNAs in WT mouse bone marrow samples (n=10), using the same data used in Panels B and C. Dnmt3a has a normalized expression value of 58.5+/−6.9 CPM (counts per million), vs. 10.8+/−1.8 CPM for Dnmt3b.

FIG. 7(A-G) is an exemplary embodiment of the effects of retroviral addback ex vivo in primary 3a KO bone marrow cells in accordance with the present disclosure. FIG. 7A: Schematic workflow for an ex vivo addback experiment using Dnmt3a^{-/-} primary mouse bone marrow cells. FIG. 7B: ProteinSimple “Western blot” showing DNA methyltransferase protein abundance in Dnmt3a^{-/-} lineage depleted bone marrow cells, 2 days after transduction. FIG. 7C: Fraction of transduced (i.e., GFP+) Dnmt3a^{-/-} bone marrow cells at 2 and 14 days after transduction. FIG. 7D: Heatmap showing the methylation values for 10,724 DMRs (from FIG. 3A) defined by comparing WGBS data from WT (n=9) versus 3a KO (n=4) samples. DNA methylation values for the same DMRs were plotted passively for purified GFP⁺ cells from the indicated retroviral vectors into Dnmt3a^{-/-} bone marrow progenitors, after 14 days in liquid culture. “3A,” DNMT3A1 cDNA-expressing vector; “3B1,” DNMT3B1 cDNA vector; “3B3,” DNMT3B3 cDNA vector; “R882H,” DNMT3A1^{R882H} cDNA vector. EV, empty vector (i.e., no cDNA inserted). FIG. 7E: Mean methylation values for all of the 3a KO DMRs in WT, 3a KO, and transduced 3a KO samples from the WGBS data shown in FIG. 7D. FIG. 7F: Extent of methylation correction (i.e., the methylation difference between addback and 3a KO scaled by the methylation difference between WT and 3a KO) for all the 3a KO DMRs in non-transduced or transduced 3a KO samples. The vertical line at 1.0 indicates remethylation of DMRs to WT levels. FIG. 7G: Extent of remethylation of Dnmt3a^{-/-} DMRs in DNMT3A1-transduced 3a KO bone marrow cells versus DNMT3B1-transduced Dnmt3a^{-/-} bone marrow cells.

FIG. 8(A-G) is an exemplary embodiment of the effects of retroviral addback ex vivo in primary 3b KO bone marrow cells in accordance with the present disclosure. FIG. 8A: Schematic workflow for an ex vivo addback experiment using 3b KO primary mouse bone marrow cells. FIG. 8B: ProteinSimple Western blot showing DNA methyltransferase protein abundance in 3b KO lineage depleted bone marrow cells, 2 days after transduction. FIG. 8C: Percentage of transduced, GFP+3b KO bone marrow cells at 2 and 14 days after transduction. FIG. 8D: Heatmap showing the methylation values for 2012 DMRs (from FIG. 3B) defined by comparing WGBS data from WT (n=9) versus 3b KO (n=3) samples. DNA methylation values for the same DMRs were plotted passively for purified GFP⁺ cells from the indicated retroviral vectors into 3b KO bone marrow progenitors, after 14 days in liquid culture. FIG. 8E: Mean methylation values for all the 3b KO DMRs in WT, 3b KO, and transduced 3b KO samples from the WGBS data shown in FIG. 8D. FIG. 8F: Extent of correction of methylation values for all the 3b KO DMRs in non-transduced or transduced 3b KO samples. The vertical line drawn at 1.0 indicates the level of methylation for these DMRs in WT bone marrow cells. FIG. 8G: Extent of remethylation of 3b KO DMRs in DNMT3A1-transduced 3b KO bone marrow cells, versus DNMT3B1-transduced 3b KO bone marrow cells.

FIG. 9 is an exemplary embodiment of DNMT3A, DNMT3B3, and DNMT3L abundance in unmanipulated vs. cultured WT bone marrow cells in accordance with the present disclosure. Protein Simple “western blot” showing DNA methyltransferase protein abundance in WT whole bone marrow cells that were unmanipulated (i.e., obtained directly from a mouse, first 3 lanes), or lineage depleted cells from unmanipulated marrow (next 3 lanes), or lineage depleted cells that were cultured for 14 days in “transplant media” ex vivo (last 3 lanes). The levels of DNMT3A and DNMT3B3 cannot be directly compared, since the antibodies used to detect these proteins may have different affinities for their cognate targets. Lin−: lineage depleted.

FIG. 10(A-G) is an exemplary embodiment of the effects of retroviral addback ex vivo in primary DKO bone marrow cells in accordance with the present disclosure. FIG. 10A: Schematic workflow for an ex vivo addback experiment using DKO primary mouse bone marrow cells. FIG. 10B: ProteinSimple Western blot showing DNA methyltransferase protein abundance in DKO lineage depleted bone marrow cells, 2 days after transduction. FIG. 10C: Percentage of transduced, GFP+ DKO bone marrow cells at 2 and 14 days after transduction. FIG. 10D: Heatmap showing the methylation values for 23,411 DMRs (from FIG. 3E) defined by comparing WGBS data from WT (n=9) versus DKO (n=3) samples. DNA methylation values for the same DMRs were plotted passively for purified GFP+ cells from the indicated retroviral vectors into DKO bone marrow progenitors, after 14 days in liquid culture. FIG. 10E: Mean methylation values for all the DKO DMRs in WT, DKO, and transduced DKO samples from the WGBS data shown in FIG. 10D. FIG. 10F: Extent of correction of methylation values for all the DKO DMRs in non-transduced or transduced DKO samples. The vertical line drawn at 1.0 indicates the level of methylation for these DMRs in WT bone marrow cells. FIG. 10G: Extent of remethylation of DKO DMRs in DNMT3A1-transduced DKO bone marrow cells versus DNMT3B1-transduced DKO bone marrow cells.

FIG. 11(A-C) is an exemplary embodiment of DNMT3A activity is augmented by copurified DNMT3B3 or DNMT3L in accordance with the present disclosure. FIG. 11A: Normalized, relative abundance of DNMT3A, DNMT3B, and DNMT3L in purified protein preparations from transfected NIH3T3 cells. DNMT3A was purified on Nickel resin columns by virtue of a 5×His tag fused in frame to human DNMT3A1 cDNA. Plasmids containing human DNMT3B3 and DNMT3L cDNAs did not have His tags. Their copurification was therefore caused by the “pulldown” of these proteins by DNMT3A. Protein abundance was quantified by mass spectrometry after tryptic digestion of the purified protein preparations. FIG. 11B: In vitro methylation assay of purified DNMT3A1 (3A) from cells transfected with DNMT3A only, or co-transfected with DNMT3B (3A/3B), or DNMT3L (3A/3L). Cells transfected with DNMT3B3 or DNMT3L only had no methyltransferase activity above background. Equal amounts of purified DNMT3A protein (defined by western blot-defined immunoreactive DNMT3A levels) were used to determine the methyltransferase activity. The activity of WT DNMT3A co-purified with DNMT3B3 is 1.49× compared with WT alone, and 5.47× when co-purified with DNMT3L.

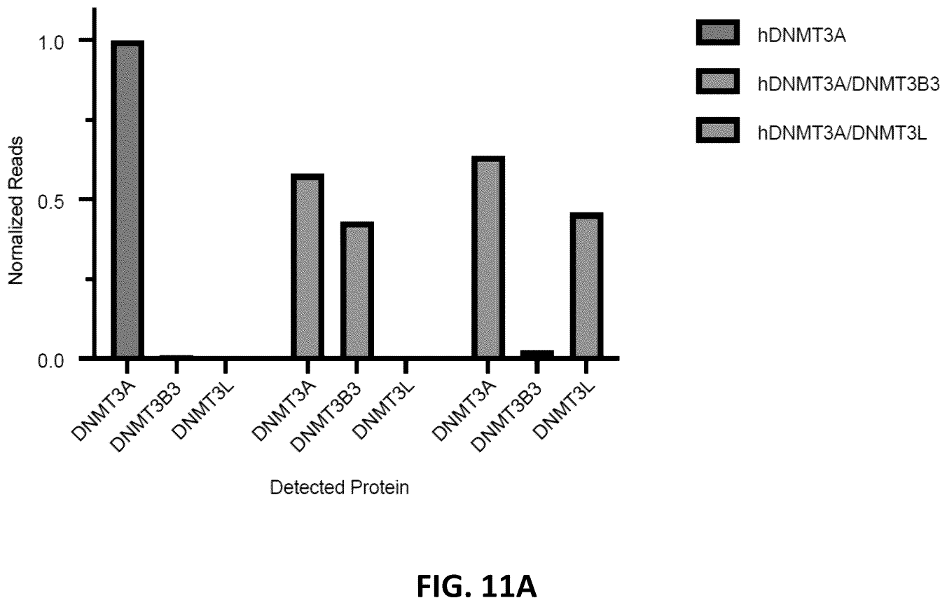

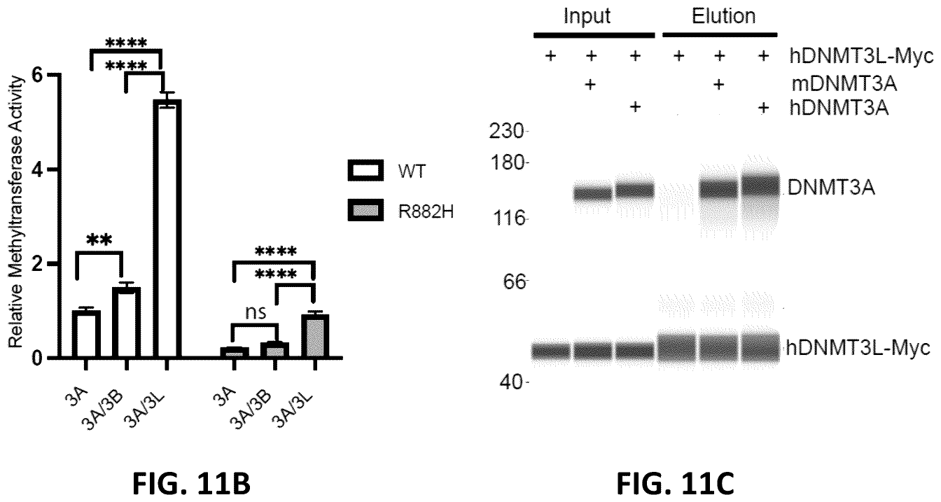

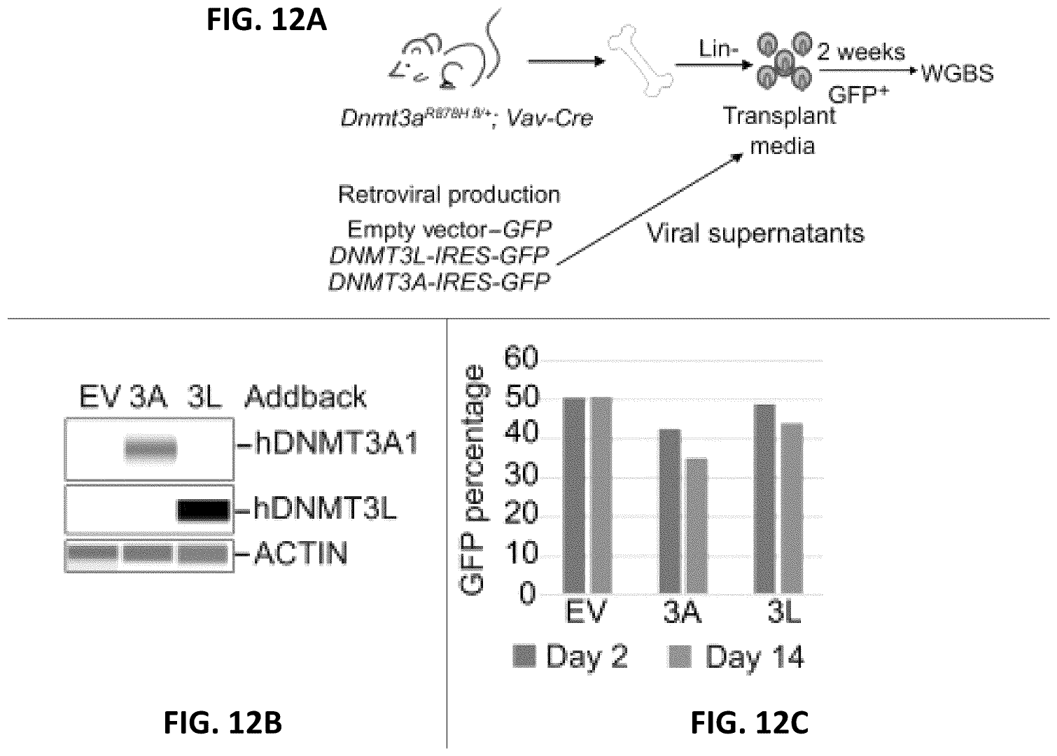

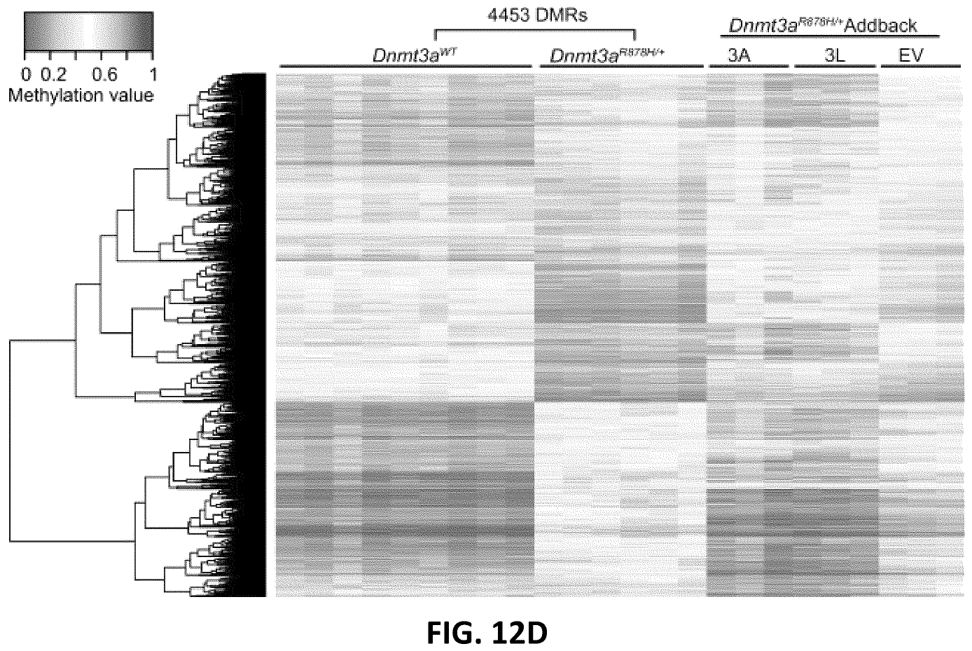

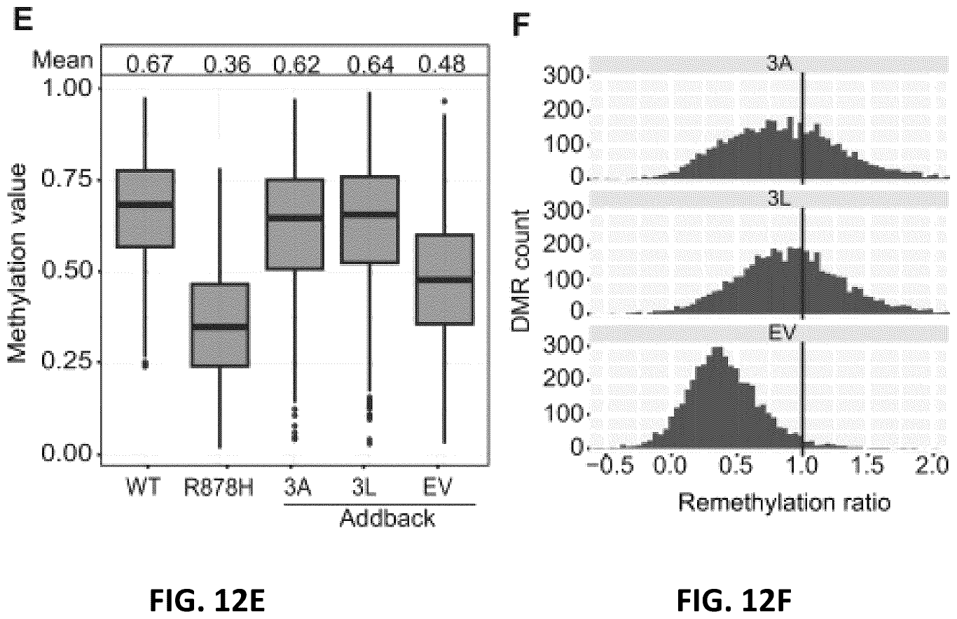

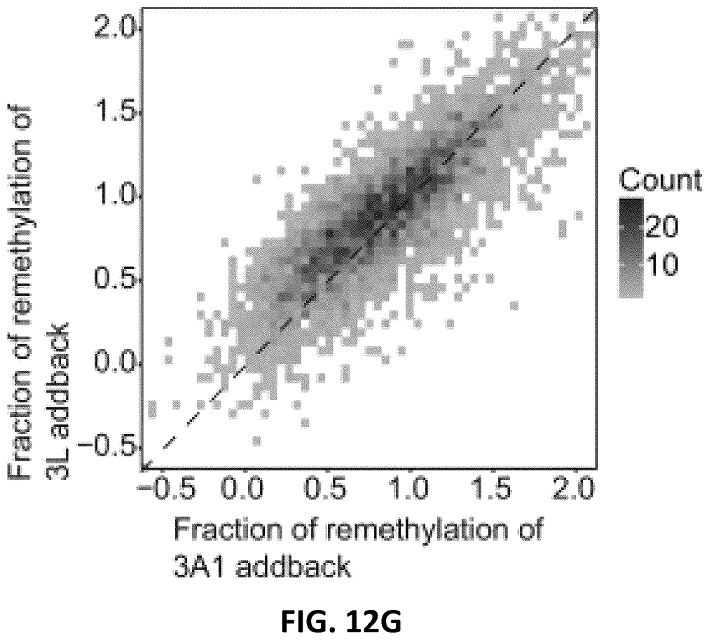

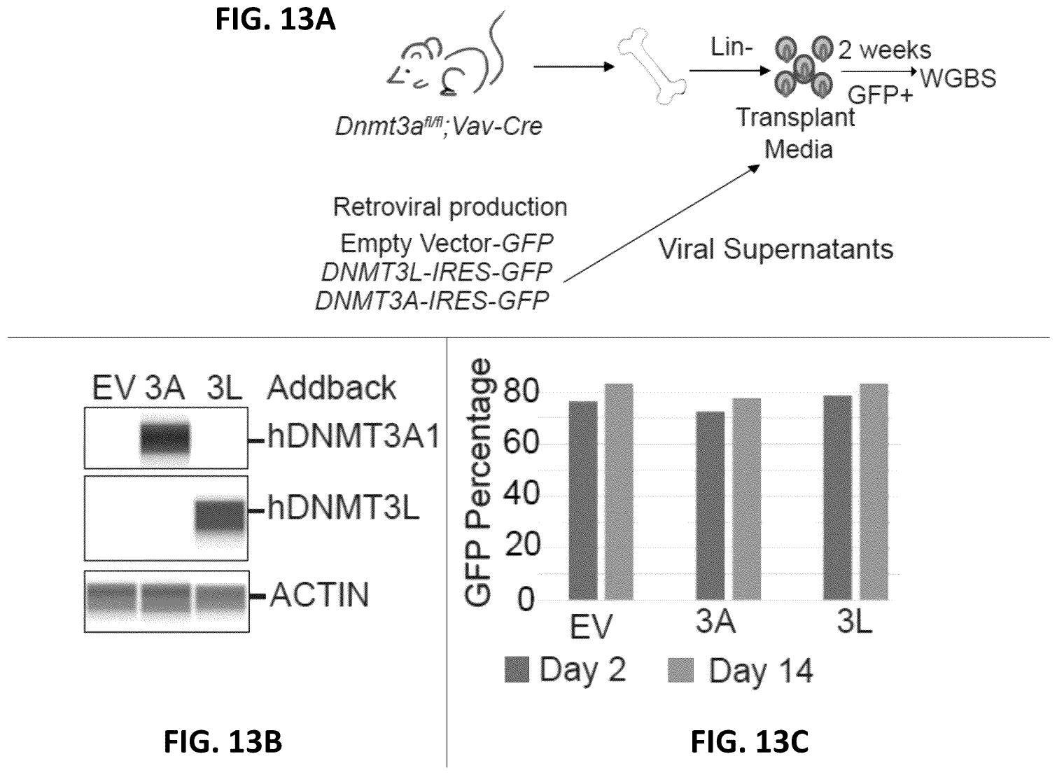

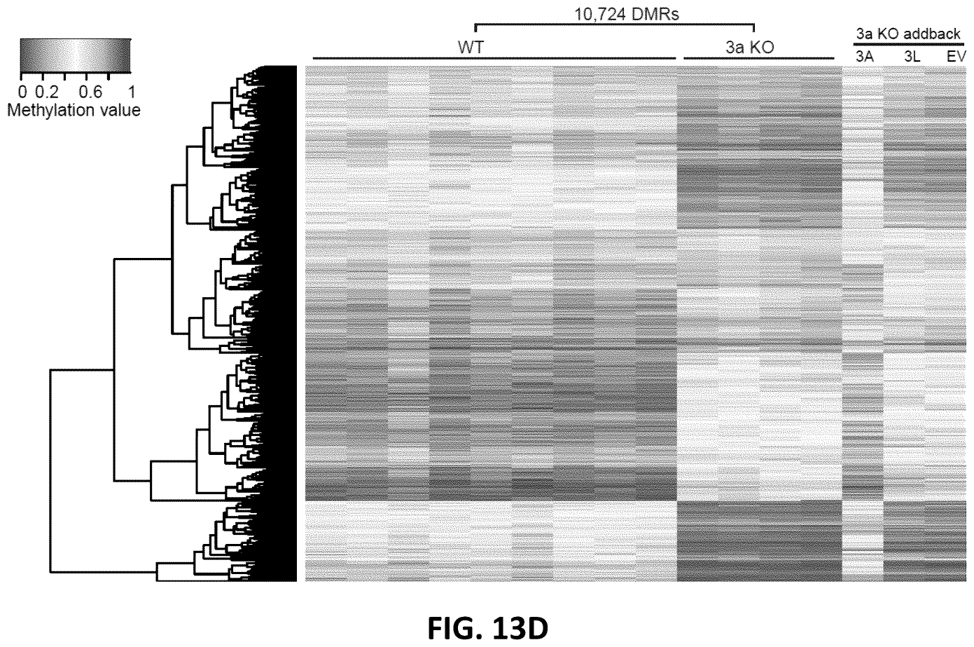

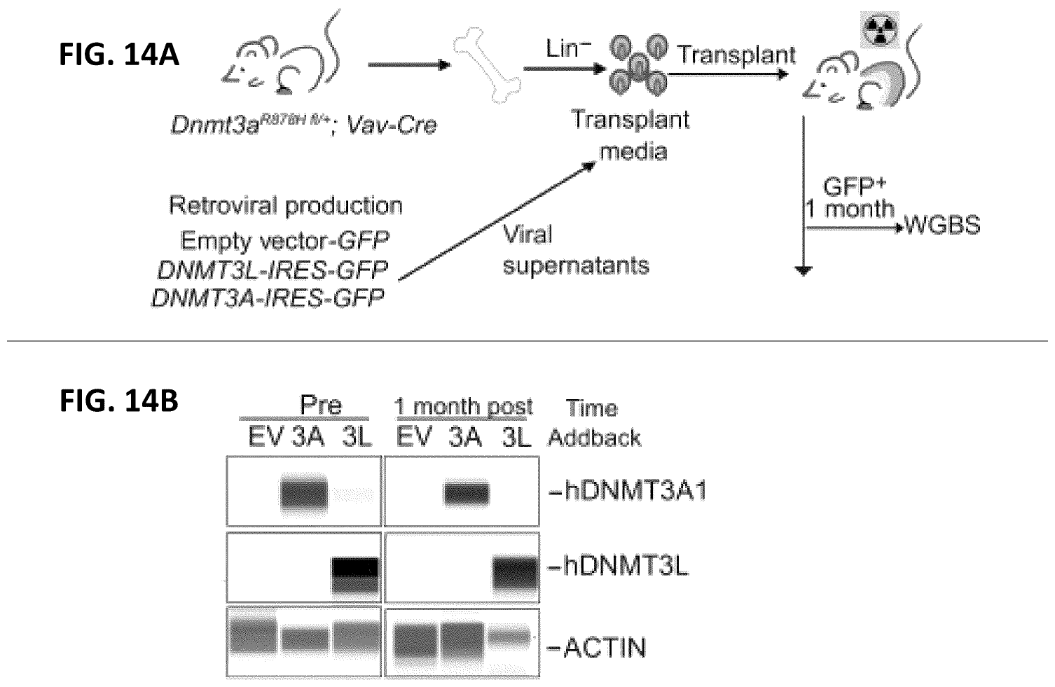

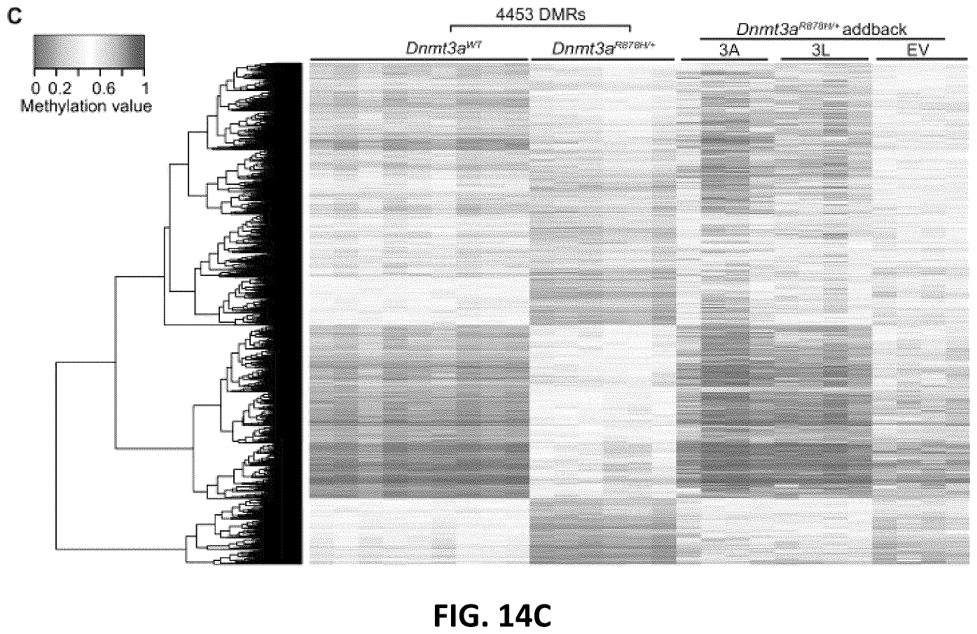

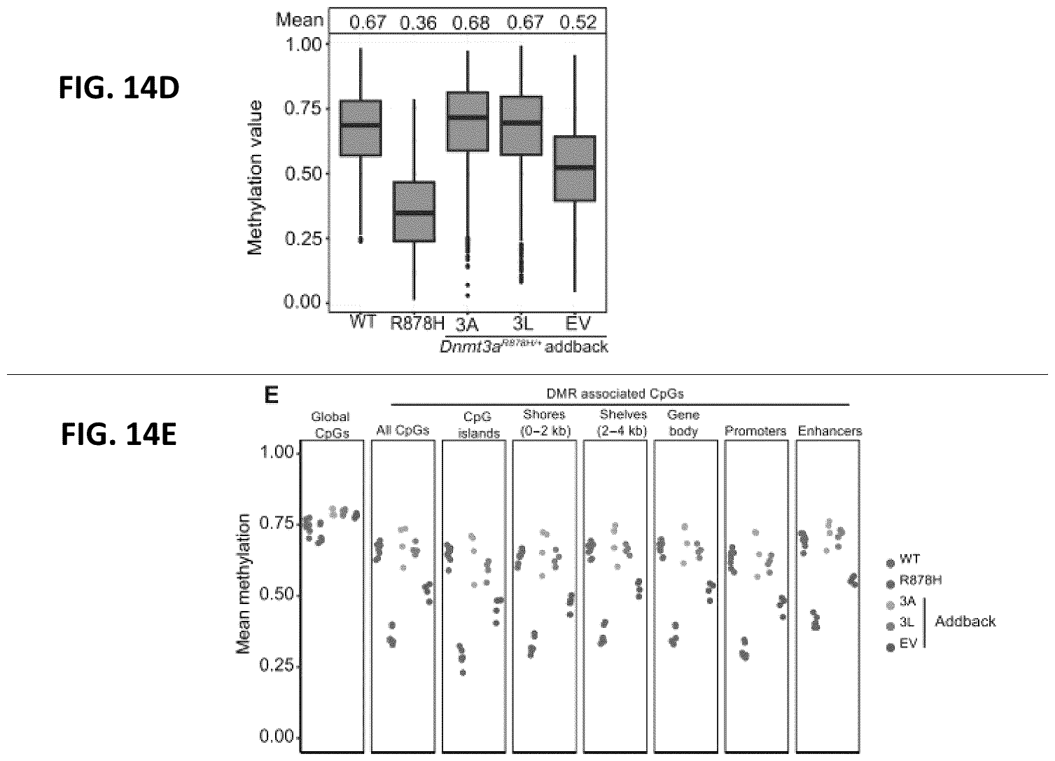

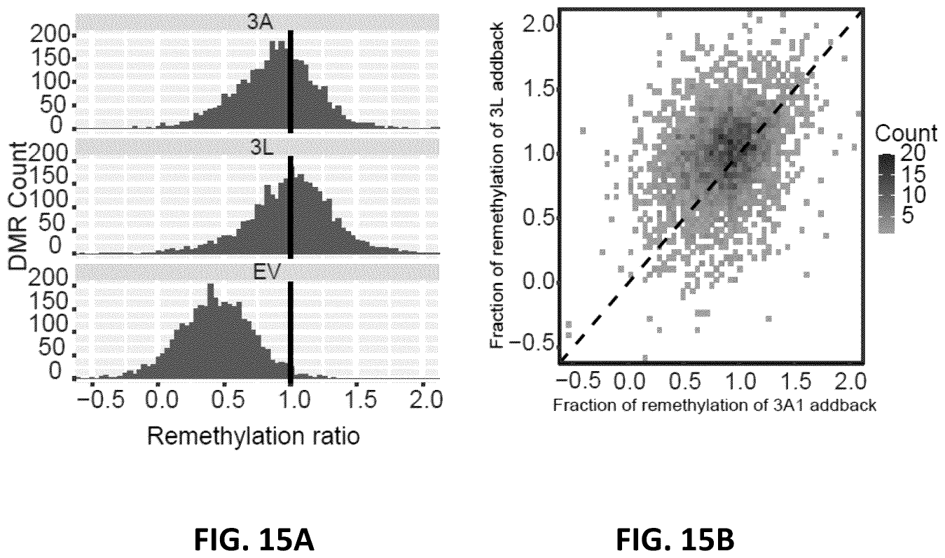

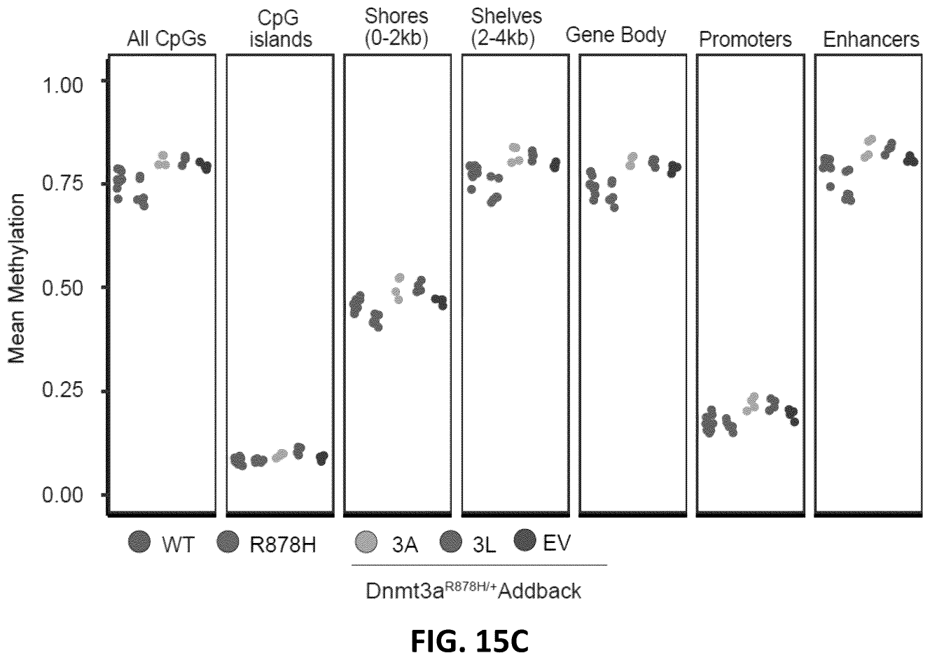

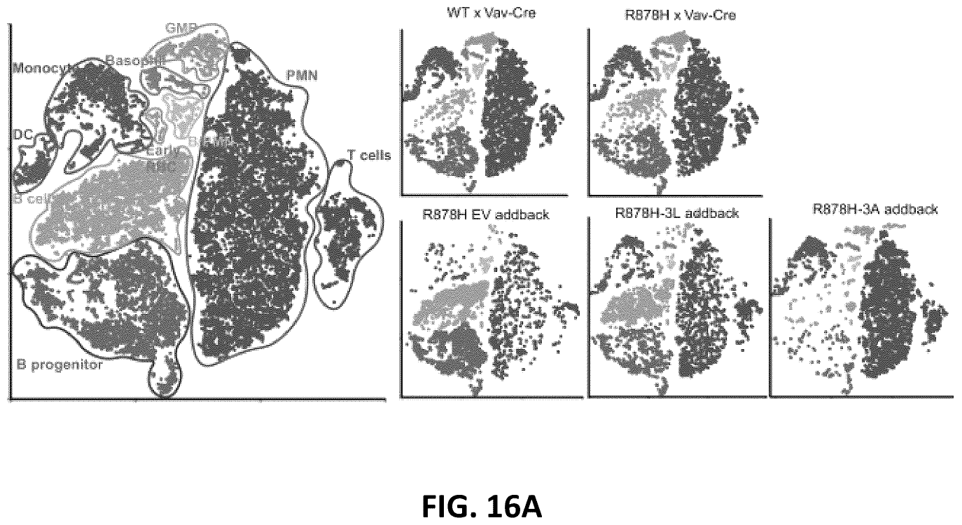

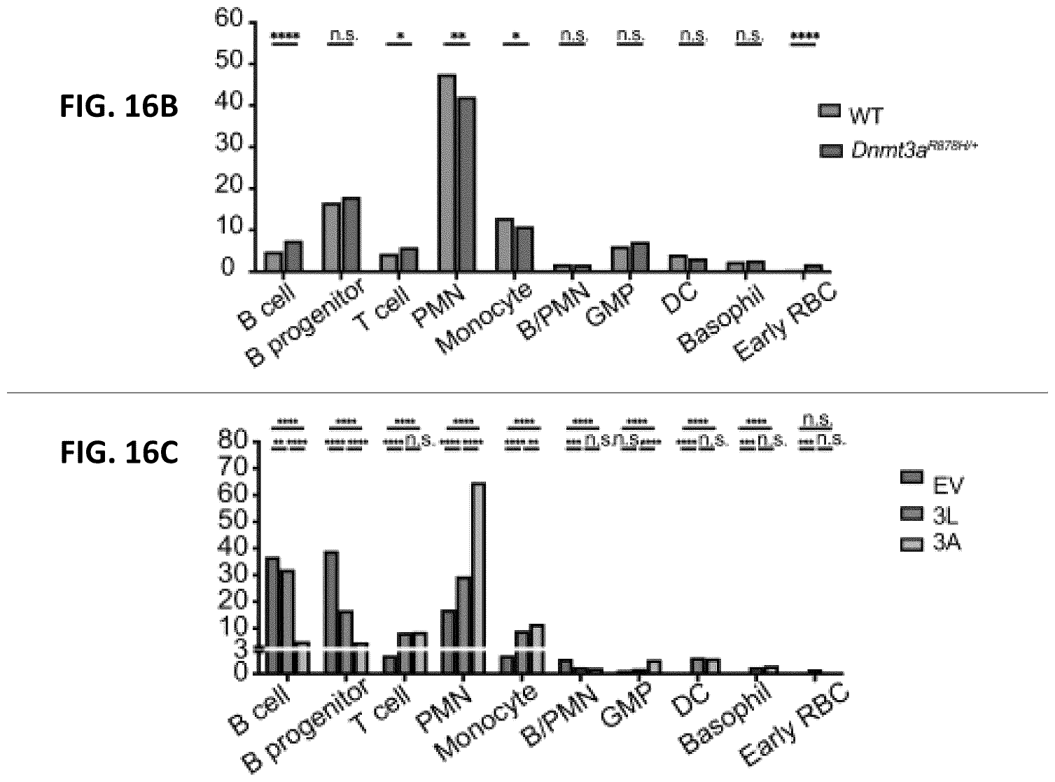

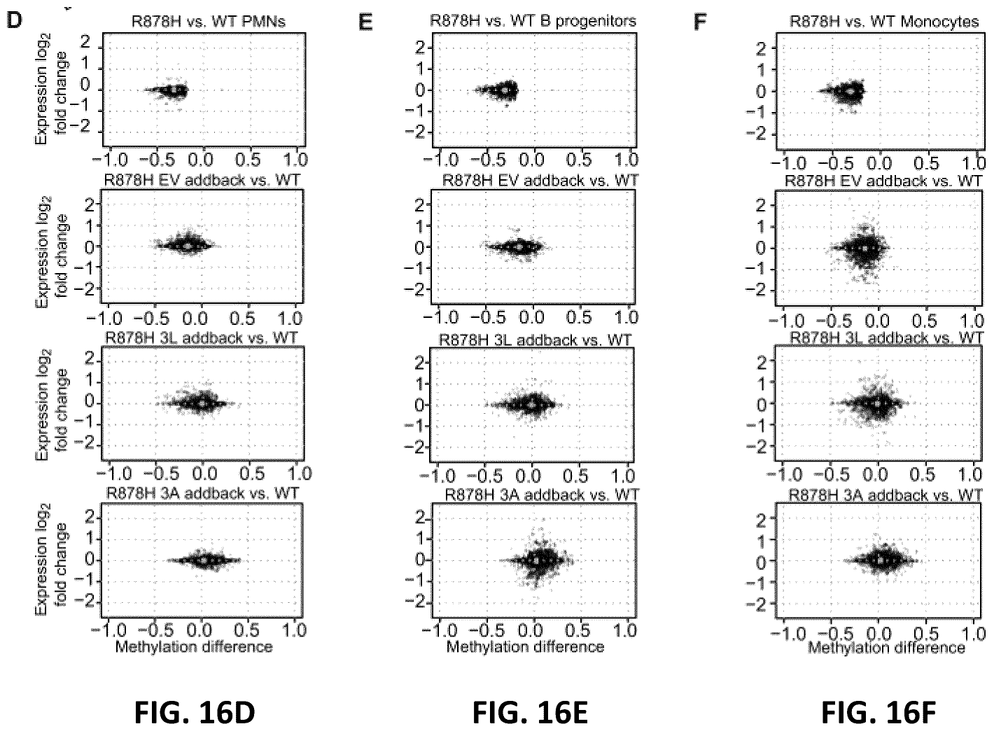

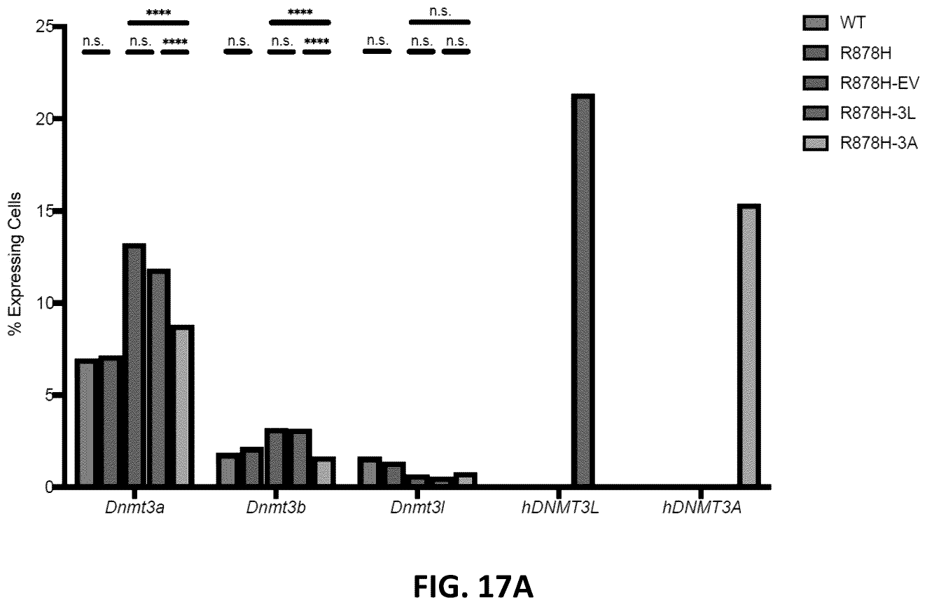

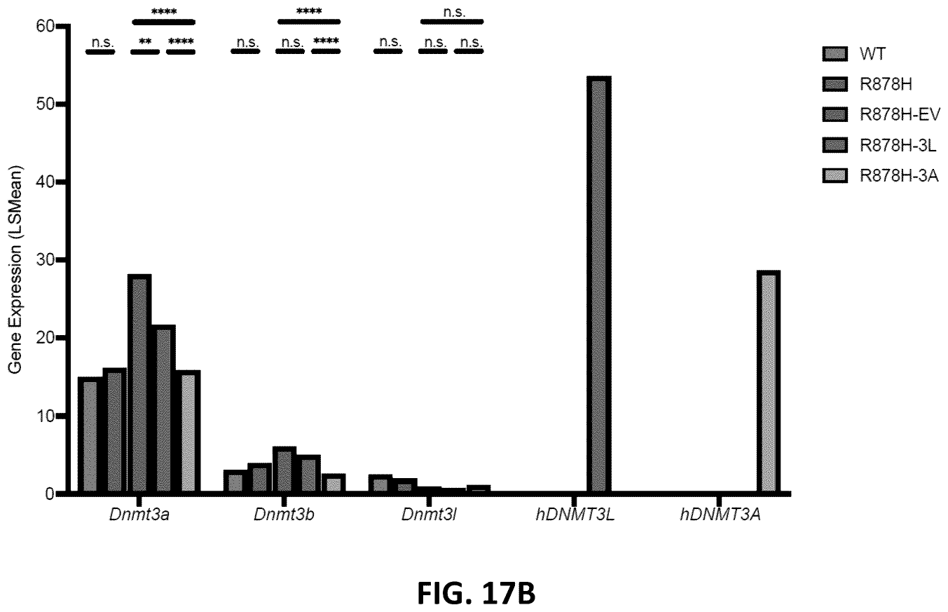

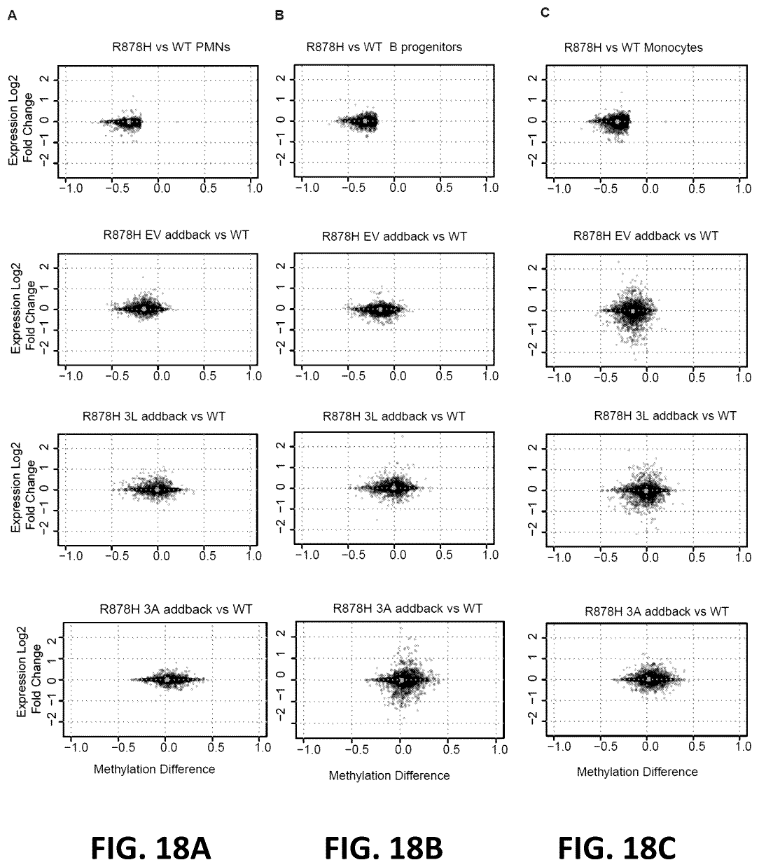

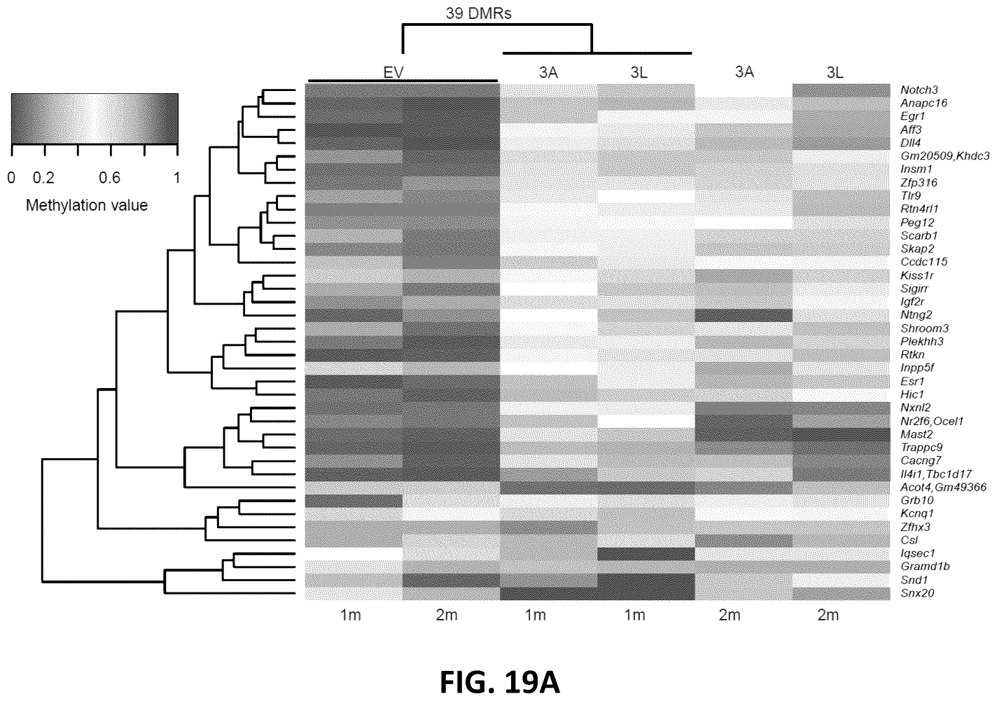

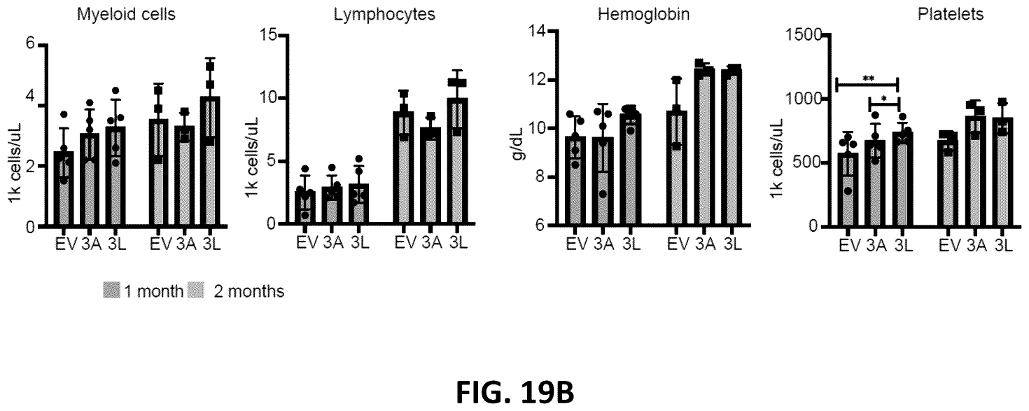

The activity of R882H DNMT3A co-purified with DNMT3B3 is 1.45× compared with R882H alone, and 4.16× when co-purified with DNMT3L. Hypothesis testing was performed using an ordinary one-way ANOVA test to compare each group of purified proteins' activity. ns p. 0.05; ** p≤0.005; **** p≤0.001. FIG. 11C: Immunoprecipitation studies of Myc-tagged human DNMT3L in K562 human cells. Cells were co-electroporated with full length human or mouse DNMT3A1 cDNAs, and MYC-tagged human DNMT3L cDNA as indicated, and 24 hours later, protein extracts were immunoprecipitated using an antibody directed against the MYC tag. Western blots were then performed with the Protein Simple system using an antibody specific for the MYCtag to identify DNMT3L (red), or an antibody that interacts with either human or mouse DNMT3A (black). Protein input levels are shown in the left panel (“Input”), and proteins pulled down with the anti-MYC antibody are shown in the right panel (“Elution”).

FIG. 12(A-G) is an exemplary embodiment of the effects of retroviral addback ex vivo in primary R878H bone marrow cells in accordance with the present disclosure. FIG. 12A: Schematic workflow for an ex vivo addback experiment using R878H primary mouse bone marrow cells. FIG. 12B: ProteinSimple Western blot showing DNA methyltransferase protein abundance in R878H lineage depleted bone marrow cells, 2 days after transduction. FIG. 12C: Percentage of transduced, GFP+R878H bone marrow cells at 2 and 14 days after transduction. FIG. 12D: Heatmap showing the methylation values for 4453 DMRs defined by comparing WGBS data from WT (n=9) versus R878H (n=6) samples. DNA methylation values for the same DMRs were plotted passively for purified GFP+ cells (i.e., transduced) from the indicated retroviral vectors into R878H bone marrow progenitors, after 14 days in liquid culture. “3L,” DNMT3L. FIG. 12E: Mean methylation values for all the R878H DMRs in WT, R878H, and transduced R878H samples from the WGBS data shown in FIG. 12D. FIG. 12F: Extent of correction of methylation values for all the R878H DMRs in non-transduced or transduced R878H samples. The vertical line drawn at 1.0 indicates the level of methylation for these DMRs in WT bone marrow cells. FIG. 12G: Extent of remethylation of R878H DMRs in DNMT3A1-transduced R878H bone marrow cells versus DNMT3L-transduced R878H bone marrow cells.

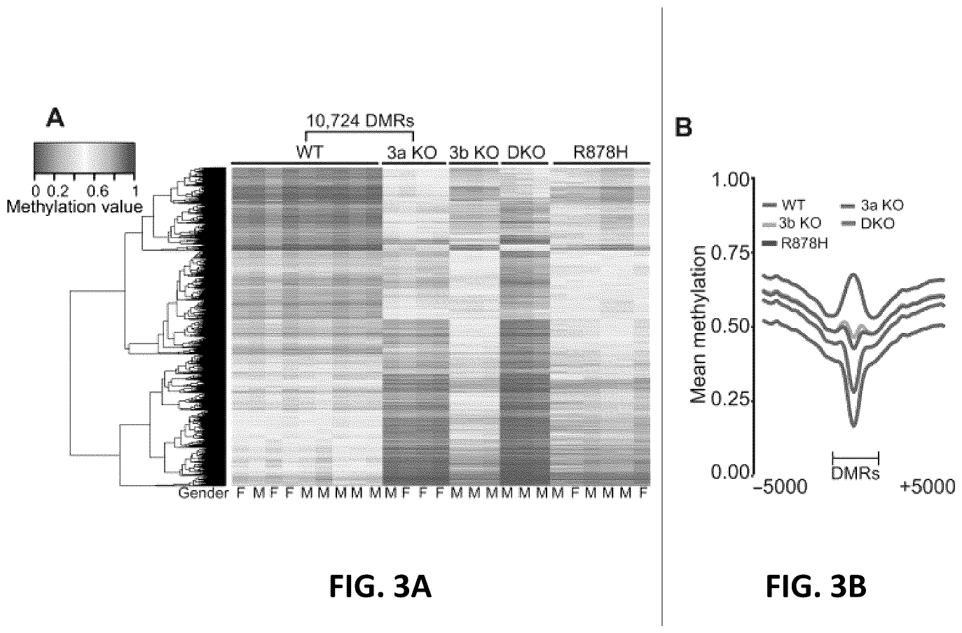

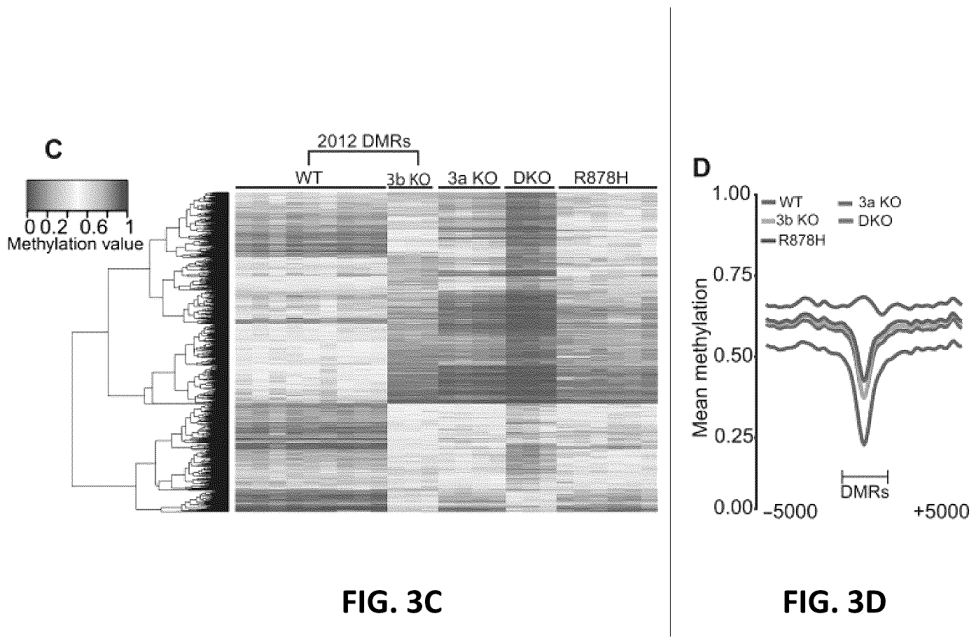

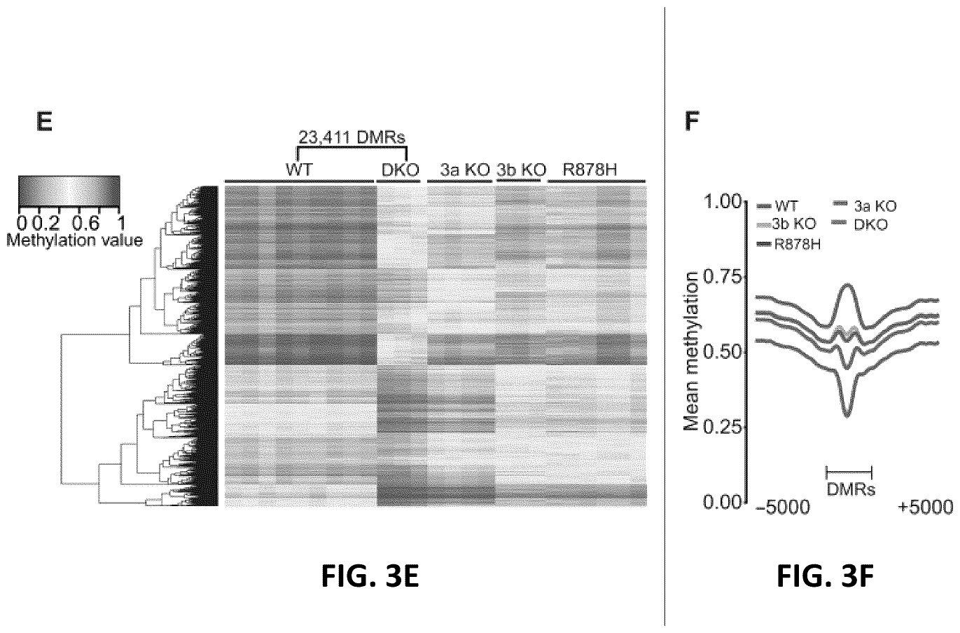

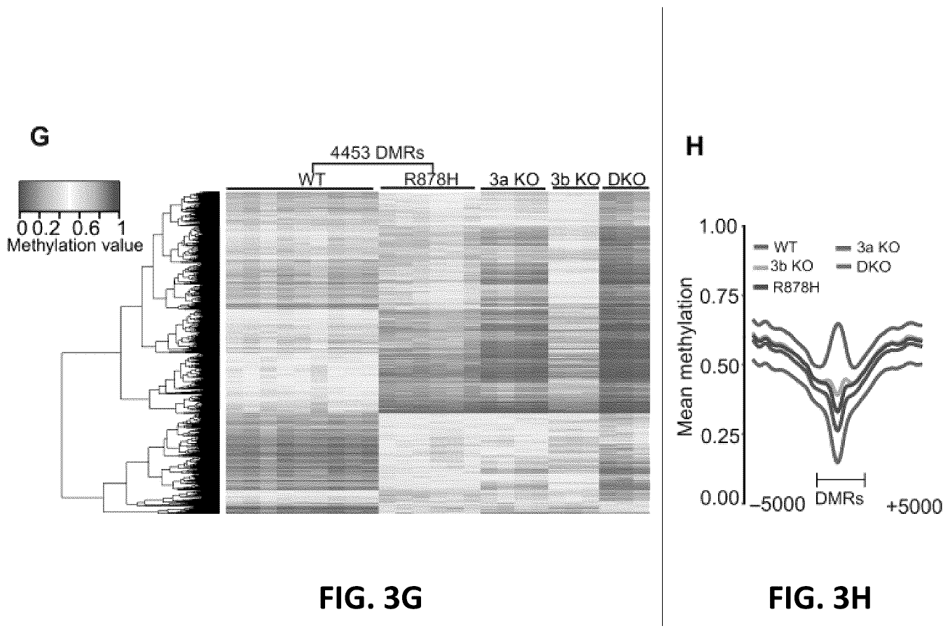

FIG. 13(A-D) is an exemplary embodiment of the effects of retroviral addback ex vivo in primary Dnmt3a^{flox/flox}×Vav1-Cre bone marrow cells in accordance with the present disclosure. FIG. 13A: Schematic workflow for an ex vivo addback experiment using Dnmt3a^{-/-} primary mouse bone marrow cells. FIG. 13B: Protein Simple western blot showing DNA methyltransferase protein abundance in lineage depleted 3a KO bone marrow cells two days post transduction. FIG. 13C: Percentage of GFP+3a KO bone marrow cells at 2 days and 14 days post transduction. FIG. 13D: Heatmap showing the methylation values for 10,724 DMRs (from FIG. 3A) defined by comparing WGBS data from WT (n=9) vs. 3a KO (n=4) samples. DNA methylation values for the same DMRs were plotted passively for purified GFP+ cells (i.e., transduced) from the indicated retroviral vectors into 3a KO bone marrow progenitors, after 14 days in liquid culture. “3A”=DNMT3A1 cDNA vector. “3L”=DNMT3L cDNA vector. “EV”=empty vector. Note that DNMT3L has no intrinsic methyltransferase activity in bone marrow cells, in the absence of DNMT3A.

FIG. 14(A-E) is an exemplary embodiment of the effects of retroviral addback in vivo with R878H bone marrow cells in accordance with the present disclosure. FIG. 14A: Schematic workflow for in vivo addback experiment using R878H mouse bone marrow cells. FIG. 14B: ProteinSimple Western blot showing DNA methyltransferase protein abundance in R878H lineage depleted bone marrow cells 2 days after transduction (Pre) and 1 month after transplantation of GFP⁺ bone marrow cells. FIG. 14C: Heatmap showing the methylation values for individual samples of 4453 DMRs defined by comparing WGBS data from WT (n=9) versus R878H (n=6) samples. Values for the same DMRs were plotted passively for R878H samples that were retrovirally transduced, transplanted to recipient mice, and harvested at 1 month after transplantation. WGBS was performed on purified GFP+ cells from each mouse. FIG. 14D: Mean methylation values for the R878H DMRs in WT, R878H, and transduced R878H samples from WGBS data used in FIG. 14C. FIG. 14E: Mean methylation values of CpGs from WGBS of WT, R878H, and addback samples. Mean values for all CpGs and DMR associated CpGs in annotated regions of the genome are shown.

FIG. 15(A-C) is an exemplary embodiment of the effects of retroviral addback in vivo in R878H bone marrow cells in accordance with the present disclosure. FIG. 15A: Extent of correction of methylation values for all the R878H DMRs in non-transduced or transduced R878H samples. The vertical line drawn at 1.0 indicates remethylation to wild-type levels. FIG. 15B: Extent of remethylation of R878H DMRs in DNMT3A1 transduced Dnmt3a^{R878H/+} bone marrow cells vs. DNMT3L transduced R878H bone marrow cells. FIG. 15C: Mean CpG methylation levels from WGBS of bone marrow cells from WT, R878H, and transduced R878H samples. Mean values for all CpGs and designated regions of the genome are shown.

FIG. 16(A-F) is an exemplary embodiment of the effects of DNMT3L or DNMT3A addback on cellular populations and gene expression in R878H bone marrow in accordance with the present disclosure. FIG. 16A: t-SNE projections of merged and separate scRNA-seq data from unmanipulated WT versus R878H bone marrow cells from 2-month-old mice or from retrovirally transduced (EV, DNMT3L, or DNMT3A), GFP+R878H bone marrow cells 1 month after transplantation, as in FIG. 14(A-E). Cell populations were assigned according to the Haemopedia algorithm and graphic-based manual review. B/Mac, biphenotypic B/macrophages; early red blood cells (RBCs) include MEPs and other erythroid progenitors. B progenitor cells include pro-B cells and pre-B cells. FIG. 16B and FIG. 16C: Cell population distributions for WT, R878H, and GFP+ cells from each addback, using the scRNA-seq data in FIG. 16A. Fisher's exact test was used for statistical analysis. *P<0.05; **P<0.01; ***P<0.001; ****P<0.0001; n.s., not significant. FIG. 16D, FIG. 16E, and FIG. 16F: Top panel in each is correlation between methylation and expression differences for individual genes within 1 kb of a DMR defined by the comparison of R878H and WT bone marrow. Panels below represent the differences between WT values at these DMRs, and the addback samples for EV, DNMT3L, or DNMT3A. X axes: Methylation differences at DMRs, showing the difference between WT and addback values for each DMR. Y axes: expression differences for genes within 1 kb of these DMRs, comparing the values in WT cells and addback cells. FIG. 16D: Methylation versus expression differences in PMNs, FIG. 16E: methylation versus expression differences in B cell progenitors, and FIG. 16F: methylation versus expression differences in monocytes.

FIG. 17(A-B) is an exemplary embodiment of endogenous and exogenous expression levels of Dnmt3a, Dnmt3b, and Dnmt3l, in WT, Dnmt3a^{R878/+}, and addback bone marrow cells from scRNA-seq data in accordance with the present disclosure. FIG. 17A: Fraction of bone marrow cells expressing endogenous mouse Dnmt3a, Dnmt3b, or Dnmt3l, vs. exogenous, retrovirally expressed human DNMT3A and DNMT3L in the same samples. Addback data was obtained from the scRNA-seq data shown in FIG. 16(A-F), obtained one month after retroviral transduction and engraftment in mice. FIG. 17B: Relative gene expression values in expressing cells for the same genes in the same samples.

FIG. 18(A-C) is an exemplary embodiment of DNA methylation and gene expression in R878H in vivo addback BM samples in accordance with the present disclosure. Top panel in each is correlation between methylation and expression differences for individual genes within 10 Kb of a DMR defined by the comparison of R878H and WT bone marrow. Panels below represent the differences between WT values at these DMRs, and the addback samples for EV, DNMT3L, or DNMT3A. X axes: methylation differences at DMRs, showing the difference between WT and addback values for each DMR. Y axes: expression differences for genes within 10 Kb of these DMRs, comparing the values in WT cells and addback cells. FIG. 18A: methylation vs. expression differences in PMNs, FIG. 18B: methylation vs. expression differences in B cell progenitors, and FIG. 18C: methylation vs. expression differences in monocytes.

FIG. 19(A-B) is an exemplary embodiment of the effects of retroviral addback on DNA methylation in WT bone marrow cells in vivo in accordance with the present disclosure. FIG. 19A: Heatmap showing the methylation values for 39 DMRs defined by comparing pooled 1 month and 2-month EV-GFP addback samples, to 1-month DNMT3A1-GFP or DNMT3L-GFP retroviral addback samples transduced into WT bone marrow cells. DNA methylation values for the same DMRs were passively plotted for the 3A and 3L addback samples harvested 2 months after transduction. WGBS was performed on the GFP+ cells purified from the bone marrow samples from each vector at each time point. “EV”=empty vector. “3A”=DNMT3A1 cDNA vector. “3L”=DNMT3L cDNA vector. FIG. 19B: Data from the complete blood counts of transplanted mice from the same in vivo addback experiment. Myeloid cells include neutrophils, monocytes, eosinophils, and basophils. * indicates p<0.05, and ** indicates p<0.01, determined by an ANOVA test.

### DETAILED DESCRIPTION OF THE DISCLOSURE

The present disclosure is based, at least in part, on the discovery that retroviral add back of DNMT3L restores DNMT3A activity in acute myeloid leukemia (AML). Further, retroviral add back has been performed in mouse AML samples initiated by the DNMT3A R878H mutation, and it was found that these cells are slowly cleared in vitro, indicating that remethylation of these cells results in their demise. As shown herein, retroviral restoration of DNMT3L in an AML mouse model results in remethylation and clearance of malignant cells (see e.g., Example 1).

### Reactivation of DNMT3L to Restore DNMT3A Activity in Disease States

The present disclosure provides for a therapeutic platform technology directed to reactivating a gene called DNMT3L. DNMT3L is developmentally silenced in all adult somatic tissues, but it is believed that pharmacological reactivation of DNMT3L via Romidepsin (Bristol Myer Squib) or a functionally similar HDAC inhibitor can promote nearly total cancer cell death in approximately 25% of AML cases, while sparing healthy cells. Other ways to reactivate DNMT3L can include gene editing (via CRISPR-CAS9) of the cis and trans-acting elements in the DNMT3L loci or gene therapy.

Loss-of-function mutations (including dominant negative mutations) in the gene encoding the de novo DNA methyltransferase, DNMT3A, are the most common cause of clonal hematopoiesis in elderly people, and the most common initiating mutation in patients with Acute Myeloid Leukemia (AML). Mutations in this gene also initiate some B and T cell malignancies. Germline mutations in DNMT3A cause the DNMT3A Overgrowth Syndrome, a disease associated with obesity, behavioral abnormalities, and an increased risk of leukemia. All of these mutations in DNMT3A reduce the function of the enzyme, resulting in very focal, canonical reductions in the methylation of DNA at thousands of discrete locations in the genome. The precise consequences of these regions of DNA hypomethylation are not yet clear, but they precede the development of leukemia, and are thought to be important for its pathogenesis.

DNMT3A has long been known to interact with a co-factor/chaperone called DNMT3L, which is related to DNMT3A; however, it lacks a methyltransferase domain, and is inert as a DNA methyltransferase. The DNMT3L gene is expressed in embryonic tissues, where it dramatically increases the activity of DNMT3A and DNMT3B, the two de novo DNA methyltransferases. However, the DNMT3L gene is developmentally silenced in all adult tissues, including hematopoietic tissues, and leukemia cells.

As described herein, DNA hypomethylation in the bone marrow cells of mice that are DNMT3A deficient can be restored by genetic ‘add back’ of wild type human DNMT3A. Restoration of enzyme function is relatively rapid, and very accurate, resulting in the partial correction of the abnormal myeloid skewing associated with DNMT3A loss. The publication entitled “Remethylation of Dnmt3a−/− hematopoietic cells is associated with partial correction of gene dysregulation and reduced myeloid skewing” to Ketkar et al. (2020), Proc Natl Acad Sci USA, v.117(6), pp. 3123-3134, and all associated supplemental material is herein incorporated by reference as support of the present disclosure. Also shown herein is that genetic add back of DNMT3A using retroviral overexpression in mouse hematopoietic cells can also overcome the dominant negative effects of the most common DNMT3A mutation found in AML patients, R882H (in the mouse, R878H), causing the complete and accurate remethylation of bone marrow cells expressing this mutation. Remarkably, retroviral add back of DNMT3L can also overcome the effect of the dominant negative R878H mutation, and remethylate bone marrow cells—presumably by restoring DNMT3A activity. Further, retroviral add back has been performed in mouse AML samples initiated by the DNMT3A R878H mutation, and it was found that these cells are slowly cleared in vitro, indicating that remethylation of these cells results in their demise.

This provides a strong rationale for developing approaches to reactivate the DNMT3L gene in tumor cells with loss-of-function mutations in DNMT3A. The data described herein reveal that this approach would at least be relevant in AMLs initiated by DNMT3A mutations, which comprises about 25% of all cases. Further, there are hundreds of thousands of elderly patients with loss-of-function mutations in DNMT3A and clonal hematopoiesis, which increases the risk of AML development more than 100 fold, to about 1% of patients each year.

Described herein is a targeted therapy for the most common initiating event causing AML, many lymphoid malignancies, clonal hematopoiesis of the elderly, and the DNMT3A Overgrowth Syndrome.

Described herein is a method of restoring the wild-type function of a tumor suppressor gene called Dnmt3a. Loss of function mutations in Dnmt3a, especially dominant negative mutations, are the key initiating events behind acute myeloid leukemia (AML), many lymphoid malignancies, clonal hematopoiesis of the elderly, and the DNMT3A overgrowth syndrome. Therefore, this invention is ultimately a method of treatment or prophylaxis for these blood disorders.

One means of restoring Dnmt3a function in loss of function or dominant negative mutations (R882H, etc.) is an overexpression of wild type Dnmt3a gene using cancer-targeting viruses. However, a much more surprising approach involves activation of Dnmt3L, a chaperone gene that greatly increases the anti-tumor activity of haploinsufficient DNMT3A; (even with dominant negative mutations). Since Dnmt3L is developmentally silenced in all adult somatic cells, including AML, the inventors propose to re-activate Dnmt3L in one of two ways: 1) pharmacologic activation using HDAC inhibitors such as Romidepsin or certain hypomethylating agents, or 2) gene editing of cis-acting elements or trans-acting factors that represses somatic Dnmt3L expression. Re-activation of Dnmt3L in K562 leukemia cells has been shown to lead to cancer cell death.

The invention describes methods of restoring DNMT3A function, thereby contributing to a treatment or prevention of certain hematopoietic malignancies, including AML. DNMT3A function can be directly restored using gene therapy means or indirectly restored using pharmacologic compounds or gene therapy targeting the constitutively silenced chaperone Dnmt3L.

Current standard of care for AML, especially in the elderly, involves chemotherapeutic agents that disrupt DNA replication (e.g., cytarabine, anthracycline, idarubicin, mitoxantrone) or mini stem cell transplant (non-myeloablative). Nevertheless, many elderly AML patients still have difficulty tolerating chemotherapy or stem cell transplant.

### DNMT3L and DNMT3A Modulation Agents

As described herein, DNMT3L or DNMT3A expression have been implicated in various diseases, disorders, and conditions. As such, modulation of DNMT3L or DNMT3A can be used for treatment of such conditions. A DNMT3L or DNMT3A modulation agent can modulate DNMT3L expression or activity or DNMT3A expression or activity. DNMT3L or DNMT3A modulation can comprise modulating the expression of DNMT3L or DNMT3A on cells, modulating the quantity of cells that express DNMT3L or DNMT3A, or modulating the quality of the DNMT3L or DNMT3A cells.

DNMT3L or DNMT3A modulation agents can be any composition or method that can modulate DNMT3L or DNMT3A expression on cells. For example, a DNMT3L or DNMT3A modulation agent can be an activator, an inhibitor, an agonist, or an antagonist. As another example, the DNMT3L or DNMT3A modulation can be the result of gene editing.

In some embodiments, the DNMT3L or DNMT3A modulation agent restores or increases DNMT3L or DNMT3A activity or expression.

For example, the DNMT3L or DNMT3A modulation agent can be a genetic vector comprising a gene encoding DNMT3L or variants thereof.

As another example, the DNMT3L or DNMT3A modulation agent can be an HDAC1 inhibitor, such as Azacitidine or Romidepsin.

As another example, the DNMT3L or DNMT3A modulation agent can be CRISPR/Cas9 to inactivate a negative regulatory element of DNMT3L or to activate DNMT3L.

### Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The term “transfection,” as used herein, refers to the process of introducing nucleic acids into cells by non-viral methods. The term “transduction,” as used herein, refers to the process whereby foreign DNA is introduced into another cell via a viral vector.

The terms “heterologous DNA sequence”, “exogenous DNA segment”, or “heterologous nucleic acid,” as used herein, each refers to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Sequences described herein can also be the reverse, the complement, or the reverse complement of the nucleotide sequences described herein. The RNA goes in the reverse direction compared to the DNA, but its base pairs still match (e.g., G to C). The reverse complementary RNA for a positive strand DNA sequence will be identical to the corresponding negative strand DNA sequence. Reverse complement converts a DNA sequence into its reverse, complement, or reverse-complement counterpart.

| Base | Name | Bases Represented | Complementary Base |
|---|---|---|---|
| A | Adenine | A | T |
| T | Thymidine | T | A |
| U | Uridine(RNA only) | U | A |
| G | Guanidine | G | C |
| C | Cytidine | C | G |
| Y | pYrimidine | C T | R |
| R | puRine | A G | Y |
| S | Strong(3Hbonds) | G C | S* |
| W | Weak(2Hbonds) | A T | W* |
| K | Keto | T/U G | M |
| M | aMino | A C | K |
| B | not A | C G T | V |
| D | not C | A G T | H |
| H | not G | A C T | D |
| V | not T/U | A C G | B |
| N | Unknown | A C G T | N |

Complementarity is a property shared between two nucleic acid sequences (e.g., RNA, DNA), such that when they are aligned antiparallel to each other, the nucleotide bases at each position will be complementary. Two bases are complementary if they form Watson-Crick base pairs.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

An “expression vector”, otherwise known as an “expression construct”, is generally a plasmid or virus designed for gene expression in cells. The vector is used to introduce a specific gene into a target cell, and can commandeer the cell's mechanism for protein synthesis to produce the protein encoded by the gene. Expression vectors are the basic tools in biotechnology for the production of proteins. The vector is engineered to contain regulatory sequences that act as enhancer and/or promoter regions and lead to efficient transcription of the gene carried on the expression vector. The goal of a well-designed expression vector is the efficient production of protein, and this may be achieved by the production of significant amount of stable messenger RNA, which can then be translated into protein. The expression of a protein may be tightly controlled, and the protein is only produced in significant quantity when necessary through the use of an inducer, in some systems however the protein may be expressed constitutively. As described herein, Escherichia coli is used as the host for protein production, but other cell types may also be used.

In molecular biology, an “inducer” is a molecule that regulates gene expression. An inducer can function in two ways, such as:
(i) By disabling repressors. The gene is expressed because an inducer binds to the repressor. The binding of the inducer to the repressor prevents the repressor from binding to the operator. RNA polymerase can then begin to transcribe operon genes.
(ii) By binding to activators. Activators generally bind poorly to activator DNA sequences unless an inducer is present. An activator binds to an inducer and the complex binds to the activation sequence and activates target gene. Removing the inducer stops transcription. Because a small inducer molecule is required, the increased expression of the target gene is called induction.

Repressor proteins bind to the DNA strand and prevent RNA polymerase from being able to attach to the DNA and synthesize mRNA. Inducers bind to repressors, causing them to change shape and preventing them from binding to DNA. Therefore, they allow transcription, and thus gene expression, to take place.

For a gene to be expressed, its DNA sequence must be copied (in a process known as transcription) to make a smaller, mobile molecule called messenger RNA (mRNA), which carries the instructions for making a protein to the site where the protein is manufactured (in a process known as translation). Many different types of proteins can affect the level of gene expression by promoting or preventing transcription. In prokaryotes (such as bacteria), these proteins often act on a portion of DNA known as the operator at the beginning of the gene. The promoter is where RNA polymerase, the enzyme that copies the genetic sequence and synthesizes the mRNA, attaches to the DNA strand.

Some genes are modulated by activators, which have the opposite effect on gene expression as repressors. Inducers can also bind to activator proteins, allowing them to bind to the operator DNA where they promote RNA transcription. Ligands that bind to deactivate activator proteins are not, in the technical sense, classified as inducers, since they have the effect of preventing transcription.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as many as several thousand base pairs from the start site of transcription.

A “ribosome binding site”, or “ribosomal binding site (RBS)”, refers to a sequence of nucleotides upstream of the start codon of an mRNA transcript that is responsible for the recruitment of a ribosome during the initiation of translation. Generally, RBS refers to bacterial sequences, although internal ribosome entry sites (IRES) have been described in mRNAs of eukaryotic cells or viruses that infect eukaryotes. Ribosome recruitment in eukaryotes is generally mediated by the 5′ cap present on eukaryotic mRNAs.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A construct of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”. “Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A. For example, the percent identity can be at least 80% or about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

Substitution refers to the replacement of one amino acid with another amino acid in a protein or the replacement of one nucleotide with another in DNA or RNA. Insertion refers to the insertion of one or more amino acids in a protein or the insertion of one or more nucleotides with another in DNA or RNA. Deletion refers to the deletion of one or more amino acids in a protein or the deletion of one or more nucleotides with another in DNA or RNA. Generally, substitutions, insertions, or deletions can be made at any position so long as the required activity is retained.

So-called conservative exchanges can be carried out in which the amino acid which is replaced has a similar property as the original amino acid, for example, the exchange of Glu by Asp, Gln by Asn, Val by lie, Leu by lie, and Ser by Thr. For example, amino acids with similar properties can be Aliphatic amino acids (e.g., Glycine, Alanine, Valine, Leucine, Isoleucine); hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of these artificially generated polypeptide sequences, a corresponding nucleic acid molecule coding for such a modulated polypeptide can be synthesized in-vitro using the specific codon-usage of the desired host cell. “Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41 (fraction G/C content)−0.63(% formamide)−(600/1). Furthermore, the Tm of a DNA:DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transformed cells can be selected and propagated to provide recombinant host cells that comprise the expression vector stably integrated in the host cell genome.

Conservative Substitutions I

| Side Chain Characteristic | Amino Acid |
|---|---|
| Aliphatic Non-polar | G A P I L V |
| Polar-uncharged | C S T M N Q |
| Polar-charged | D E K R |
| Aromatic | H F W Y |
| Other | N Q D E |

Conservative Substitutions II

| | |
|---|---|
| Side Chain | |
| Characteristic | Amino Acid |

Non-polar (hydrophobic)

| | |
|---|---|
| A. Aliphatic: | A L I V P |
| B. Aromatic: | F W |
| C. Sulfur-containing: | M |
| D. Borderline: | G |

Uncharged-polar

| | |
|---|---|
| A. Hydroxyl: | S T Y |
| B. Amides: | N Q |
| C. Sulfhydryl: | C |
| D. Borderline: | G |
| Positively Charged (Basic): | K R H |
| Negatively Charged | D E |
| (Acidic): | |

Conservative Substituations III

| | Exemplary |
|---|---|
| Original Residue | Substitution |
| Ala (A) | Val, Leu, Ile |
| Arg (R) | Lys, Gln, Asn |
| Asn (N) | Gln, His, Lys, Arg |
| Asp (D) | Glu |
| Cys (C) | Ser |
| Gln (Q) | Asn |
| Glu (E) | Asp |
| His (H) | Asn, Gln, Lys, Arg |
| Ile (I) | Leu, Val, Met, Ala, |
| | Phe, |
| Leu (L) | Ile, Val, Met, Ala, Phe |
| Lys (K) | Arg, Gln, Asn |
| Met(M) | Leu, Phe, Ile |
| Phe (F) | Leu, Val, Ile, Ala |
| Pro (P) | Gly |
| Ser (S) | Thr |
| Thr (T) | Ser |
| Trp(W) | Tyr, Phe |
| Tyr (Y) | Trp, Phe, Tur, Ser |
| Val (V) | Ile, Leu, Met, Phe, Ala |

Exemplary nucleic acids that may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA that is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides (ASOs), protein aptamers, nucleotide aptamers, and RNA interference (RNAi) (e.g., small interfering RNAs (siRNA), short hairpin RNA (shRNA), single guide RNA (sgRNA), and micro RNAs (miRNA) (see e.g., Rinaldi and Wood (2017) Nature Reviews Neurology 14, describing ASO therapies; Fanning and Symonds (2006) Handb Exp Pharmacol. 173, 289-303G, describing hammerhead ribozymes and small hairpin RNA; Helene, et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinformatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

### Genome Editing

As described herein, DNMT3A or DNMT3L signals can be modulated (e.g., reduced, eliminated, or enhanced) using genome editing. Processes for genome editing are well known; see e.g., Aldi 2018 Nature Communications 9(1911). Except as otherwise noted herein, therefore, the process of the present disclosure can be carried out in accordance with such processes.

For example, genome editing can comprise CRISPR/Cas9, CRISPR-Cpf1, TALEN, or ZNFs. Adequate enhancement of DNMT3L activity or expression or DNMT3A enzymatic activity by genome editing can result in protection from DNMT3A-associated disease.

As an example, clustered regularly interspaced short palindromic repeats (CRISPR)/CRISPR-associated (Cas) systems are a new class of genome-editing tools that target desired genomic sites in mammalian cells. Recently published type II CRISPR/Cas systems use Cas9 nuclease that is targeted to a genomic site by complexing with a synthetic guide RNA that hybridizes to a 20-nucleotide DNA sequence and immediately preceding an NGG motif recognized by Cas9 (thus, a (N)₂₀NGG target DNA sequence). This results in a double-strand break three nucleotides upstream of the NGG motif. The double strand break instigates either non-homologous end-joining, which is error-prone and conducive to frameshift mutations that knock out gene alleles, or homology-directed repair, which can be exploited with the use of an exogenously introduced double-strand or single-strand DNA repair template to knock in or correct a mutation in the genome. Thus, genomic editing, for example, using CRISPR/Cas systems could be useful tools for therapeutic applications for DNMT3A-associated disease to target cells by the addition of DNMT3L or DNMT3A signals (e.g., activate (e.g., CRISPRa), upregulate, overexpress) or by inhibiting a regulatory element or trans-acting factor responsible for DNMT3L silencing.

For example, the methods as described herein can comprise a method for altering a target polynucleotide sequence in a cell comprising contacting the polynucleotide sequence with a clustered regularly interspaced short palindromic repeats-associated (Gas) protein.

### Gene Therapy and Genome Editing

Gene therapies can include inserting a functional gene with a viral vector. Gene therapies for DNMT3A-associated diseases are rapidly advancing.

There has recently been an improved landscape for gene therapies. For example, in the first quarter of 2019, there were 372 ongoing gene therapy clinical trials (Alliance for Regenerative Medicine, 5/9/19).

Any vector known in the art can be used. For example, the vector can be a viral vector selected from retrovirus, lentivirus, herpes, adenovirus, adeno-associated virus (AAV), rabies, Ebola, lentivirus, or hybrids thereof.

### Gene Therapy Strategies.

| | |
|---|---|
| | Associated experimental |
| Strategy | models |

Viral Vectors

| | | |
|---|---|---|
| Retroviruses | Retroviruses are RNA viruses | Murine model of MPS VII |
| | transcribing their single-stranded | Canine model of MPS VII |
| | genome into a double-stranded | |
| | DNA copy, which can integrate | |
| | into host chromosome | |
| Adenoviruses (Ad) | Ad can transfect a variety of | Murine model of Pompe, |
| | quiescent and proliferating | Fabry, Walman diseases, |
| | cell types from various species | aspartylglucosaminuria |
| | and can mediate robust | and MPS VII |
| | gene expression | |
| Adeno-associated | Recombinant AAV vectors contain | Murine models of Pompe, Fabry |
| Viruses (AAV) | no viral DNA and can carry ~4.7 kb | diseases, Aspartylglucosaminuria, |
| | of foreign transgenic material. | Krabbe disease, Metachromatic |
| | They are replication defective | leukodystrophy, MPS I, MPSII, |
| | and can replicate only while | MPSIIIA, MPSIIIB, MPSIV, |
| | coinfecting with a helper virus | MPSVI, MPS VII, CLN1, CLN2, |
| | | CLN3, CLN5, CLN6 |

Non-viral vectors

| | | |
|---|---|---|
| plasmid DNA | pDNA has many desired | Mouse model of Fabry disease |
| (pDNA) | characteristics as a gene | |
| | therapy vector; there are no limits | |
| | on the size or genetic constitution | |
| | of DNA, it is relatively | |
| | inexpensive to supply, and unlike | |
| | viruses, antibodies are not | |
| | generated against DNA in normal | |
| | individuals | |
| RNAi | RNAi is a powerful tool for gene | Transgenic mouse strain |
| | specific silencing that could | Mouse models of acute liver failure |
| | be useful as an enzyme reduction | Mice with hepatitis B virus |
| | therapy or means to promote | Fabry mouse |
| | read-through of a premature | |
| | stop codon | |

Gene therapy can allow for the constant delivery of the enzyme directly to target organs and eliminates the need for weekly infusions. Also, correction of a few cells could lead to the enzyme being secreted into the circulation and taken up by their neighboring cells (cross-correction), resulting in widespread correction of the biochemical defects. As such, the number of cells that must be modified with a gene transfer vector is relatively low.

Genetic modification can be performed either ex vivo or in vivo. The ex vivo strategy is based on the modification of cells in culture and transplantation of the modified cell into a patient. Cells that are most commonly considered therapeutic targets for monogenic diseases are stem cells. Advances in the collection and isolation of these cells from a variety of sources have promoted autologous gene therapy as a viable option.

The use of endonucleases for targeted genome editing can solve the limitations presented by the usual gene therapy protocols. These enzymes are custom molecular scissors, allowing cutting DNA into well-defined, perfectly specified pieces, in virtually all cell types. Moreover, they can be delivered to the cells by plasmids that transiently express the nucleases, or by transcribed RNA, avoiding the use of viruses.

### Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent, and consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

### Therapeutic Methods

Also provided is a process of treating, preventing, or reversing a DNMT3A deficiency-associated disease in a subject in need of administration of a therapeutically effective amount of a therapeutic agent capable of restoring or increasing DNMT3L activity or expression or increasing DNMT3A enzymatic activity, so as to result in remethylation of bone marrow cells; remethylation, depletion, or death of diseased (e.g., cancer) cells, or correction of abnormal myeloid skewing.

Methods described herein are generally performed on a subject in need thereof. A subject in need of the therapeutic methods described herein can be a subject having, diagnosed with, suspected of having, or at risk for developing a DNMT3A deficiency-associated disease. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of a therapeutic agent capable of restoring or increasing DNMT3L activity or expression or increasing DNMT3A enzymatic activity is, for example, an amount that would cause the desired therapeutic effect in a subject while minimizing undesired side effects. In various embodiments, an effective amount of a therapeutic agent capable of restoring or increasing DNMT3L activity or expression or increasing DNMT3A enzymatic activity described herein can result in remethylation of bone marrow cells; remethylation, depletion, or death of diseased (e.g., cancer) cells, or correction of abnormal myeloid skewing, and therefore substantially inhibit DNMT3A deficiency-associated disease, slow the progress of DNMT3A deficiency-associated disease, or limit the development of DNMT3A deficiency-associated disease.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration.

When used in the treatments described herein, a therapeutically effective amount of a therapeutic agent capable of restoring or increasing DNMT3L activity or expression or increasing DNMT3A enzymatic activity can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to result in remethylation of bone marrow cells; remethylation, depletion, or death of diseased (e.g., cancer) cells, or correction of abnormal myeloid skewing.

The amount of a composition described herein that can be combined with a pharmaceutically acceptable carrier to produce a single dosage form will vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing, reversing, or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of a therapeutic agent capable of restoring or increasing DNMT3L activity or expression or increasing DNMT3A enzymatic activity can occur as a single event or over a time course of treatment. For example, a therapeutic agent capable of restoring or increasing DNMT3L activity or expression or increasing DNMT3A enzymatic activity can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to or before, concurrent with, or after conventional treatment modalities for a DNMT3A deficiency-associated disease.

A therapeutic agent capable of restoring or increasing DNMT3L activity or expression or increasing DNMT3A enzymatic activity can be administered simultaneously or sequentially with another agent, such as an antibiotic, an anti-inflammatory, or another agent. For example, a therapeutic agent capable of restoring or increasing DNMT3L activity or expression or increasing DNMT3A enzymatic activity can be administered simultaneously with another agent, such as an antibiotic or an anti-inflammatory. Simultaneous administration can occur through administration of separate compositions, each containing one or more of a therapeutic agent capable of restoring or increasing DNMT3L activity or expression or increasing DNMT3A enzymatic activity, an antibiotic, an anti-inflammatory, or another agent. Simultaneous administration can occur through administration of one composition containing two or more of a therapeutic agent capable of restoring or increasing DNMT3L activity or expression or increasing DNMT3A enzymatic activity, an antibiotic, an anti-inflammatory, or another agent. A therapeutic agent capable of restoring or increasing DNMT3L activity or expression or increasing DNMT3A enzymatic activity can be administered sequentially with an antibiotic, an anti-inflammatory, or another agent. For example, a therapeutic agent capable of restoring or increasing DNMT3L activity or expression or increasing DNMT3A enzymatic activity can be administered before or after administration of an antibiotic, an anti-inflammatory, or another agent.

Active compounds are administered at a therapeutically effective dosage sufficient to treat a condition associated with a condition in a patient. For example, the efficacy of a compound can be evaluated in an animal model system that may be predictive of efficacy in treating the disease in a human or another animal, such as the model systems shown in the examples and drawings.

An effective dose range of a therapeutic can be extrapolated from effective doses determined in animal studies for a variety of different animals. In general, a human equivalent dose (HED) in mg/kg can be calculated in accordance with the following formula (see e.g., Reagan-Shaw et al., FASEB J., 22(3):659-661, 2008, which is incorporated herein by reference):

$$H E D ( mg / kg ) = Animal dose ( mg / kg ) \times ( Animal {K}_{m} / Human {K}_{m} )$$

Use of the Kₘ factors in conversion results in more accurate HED values, which are based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are peculiar to each individual. Nonetheless, a calculated HED dose provides a general guide. Other factors affecting the dose include the physical and clinical state of the patient, the route of administration, the intended goal of treatment, and the potency, stability, and toxicity of the particular therapeutic formulation.

The actual dosage amount of a compound of the present disclosure or composition comprising a compound of the present disclosure administered to a subject may be determined by physical and physiological factors such as type of animal treated, age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

In some embodiments, a therapeutic agent capable of restoring or increasing DNMT3L activity or expression or increasing DNMT3A enzymatic activity may be administered in an amount from about 1 mg/kg to about 100 mg/kg, or about 1 mg/kg to about 50 mg/kg, or about 1 mg/kg to about 25 mg/kg, or about 1 mg/kg to about 15 mg/kg, or about 1 mg/kg to about 10 mg/kg, or about 1 mg/kg to about 5 mg/kg, or about 3 mg/kg. In some embodiments, a therapeutic agent capable of restoring or increasing DNMT3L activity or expression or increasing DNMT3A enzymatic activity such as a compound described herein may be administered in a range of about 1 mg/kg to about 200 mg/kg, or about 50 mg/kg to about 200 mg/kg, or about 50 mg/kg to about 100 mg/kg, or about 75 mg/kg to about 100 mg/kg, or about 100 mg/kg.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day.

In other non-limiting examples, a dose may also comprise from about 1 micro-gram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Cell Therapy

Cells generated according to the methods described herein can be used in cell therapy. Cell therapy (also called cellular therapy, cell transplantation, or cytotherapy) can be a therapy in which viable cells are injected, grafted, or implanted into a patient in order to effectuate a medicinal effect or therapeutic benefit. For example, transplanting T-cells capable of fighting cancer cells via cell-mediated immunity can be used in the course of immunotherapy, grafting stem cells can be used to regenerate diseased tissues, or transplanting beta cells can be used to treat diabetes.

Stem cell and cell transplantation has gained significant interest by researchers as a potential new therapeutic strategy for a wide range of diseases, in particular for degenerative and immunogenic pathologies.

Allogeneic cell therapy or allogenic transplantation uses donor cells from a different subject than the recipient of the cells. A benefit of an allogeneic strategy is that unmatched allogenic cell therapies can form the basis of “off the shelf” products.

Autologous cell therapy or autologous transplantation uses cells that are derived from the subject's own tissues. It could also involve the isolation of matured cells from diseased tissues, to be later re-implanted at the same or neighboring tissues. A benefit of an autologous strategy is that there is limited concern for immunogenic responses or transplant rejection.

Xenogeneic cell therapies or xenotransplantation uses cells from another species. For example, pig derived cells can be transplanted into humans. Xenogeneic cell therapies can involve human cell transplantation into experimental animal models for assessment of efficacy and safety or enable xenogeneic strategies to humans as well.

### Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intratumoral, intranasal, inhalation (e.g., in an aerosol), implanted, intramuscular, intraperitoneal, intravenous, intrathecal, intracranial, intracerebroventricular, subcutaneous, intranasal, epidural, intrathecal, ophthalmic, transdermal, buccal, and rectal.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

### Screening

Also provided are screening methods.

The subject methods find use in the screening of a variety of different candidate molecules (e.g., therapeutic candidate molecules). Candidate substances for screening according to the methods described herein include, but are not limited to, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules online; and electronic libraries of commercial compounds provided by vendors, for example, ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict the bioavailability of a compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8A to about 15A.

### Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a therapeutic agent capable of restoring or increasing DNMT3L activity or expression or increasing DNMT3A enzymatic activity, genetic vectors, etc. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline each of which has been packaged under a neutral non-reacting gas, such as nitrogen. Ampules may consist of any suitable material, such as glass, organic polymers, such as polycarbonate, polystyrene, ceramic, metal, or any other material typically employed to hold reagents. Other examples of suitable containers include bottles that may be fabricated from similar substances as ampules and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or another substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or sample, a wild-type subject or sample, or from populations thereof. A reference value can be used in place of a control or reference sample, which was previously obtained from a healthy subject or a group of healthy subjects or a wild-type subject or sample. A control sample or a reference sample can also be a sample with a known amount of a detectable compound or a spiked sample.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Reactivation of DNMT3L to Restore DNMT3A Activity in Disease States, Including Retroviral Add Back of DNMT3L

This Example describes how restoring DNMT3L expression can be used to treat several DNMT3A deficiency-associated diseases, including leukemia, acute myeloid leukemia (AML), a lymphoid malignancy, clonal hematopoiesis, and DNMT3A Overgrowth Syndrome.

Retroviral add back of DNMT3L in mouse hematopoietic cells overcame the dominant negative effects of the most common DNMT3A mutation found in AML patients, R882H (in the mouse, R878H). Specifically, restoration of DNMT3L resulted in remethylation of bone marrow cells—presumably by restoring DNMT3A activity. Further, retroviral add back in mouse AML samples initiated by the DNMT3A R878H mutation resulted in clearance of these cells in vitro, indicating that remethylation of these cells results in their demise.

These results show that reactivation of the DNMT3L gene in tumor cells with loss-of-function mutations in DNMT3A is a strong rationale for treating DNMT3A deficiency-associated diseases, such as AML initiated by DNMT3A mutations or clonal hematopoiesis.

Therefore, several approaches to reactivate the DNMT3L gene are being explored (including in human hematopoietic cells and leukemic cells):

(i) Drugs and/or small molecules can reactivate DNMT3L expression. More than 20 years ago, reactivation of DNMT3L was described using histone deacetylase (HDAC) inhibitors and/or hypomethylating agents in tissue culture cells. As described herein, Romidepsin, an HDAC1 inhibitor currently in clinical trials for AML and cutaneous T cell lymphomas, can strongly reactivate DNMT3L in K562 leukemia cells, and a reporter cell line was created to allow for screening small molecule libraries for their ability to reactivate DNMT3L. A clinical trial of Azacitidine and Romidepsin for refractory/relapsed AML patients with DNMT3A mutations with correlative scientific studies are designed to determine whether clinical responses are correlated with DNMT3L reactivation, including reactivation in AML blasts.

(ii) The DNMT3L locus is being evaluated to identify the cis-acting and trans-acting factors responsible for DNMT3L silencing in somatic tissues. The identification of a negative regulatory element may allow for restoration of DNMT3L gene activity by inactivating the element with CRISPR/Cas9 mediated gene editing approaches, e.g., by delivering these reagents specifically to AML cells in vivo.

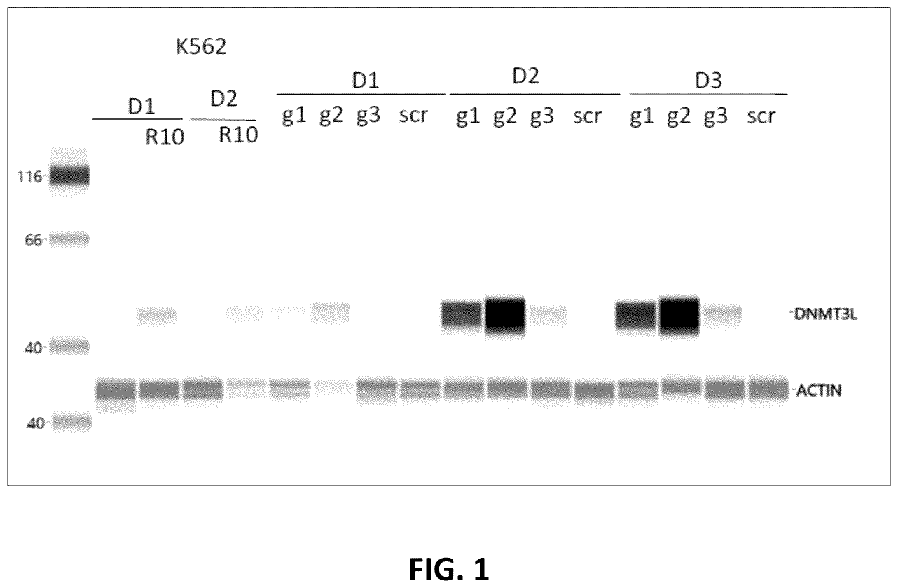

(iii) Use of CRISPR/a technology is being explored to reactivate the endogenous DNMT3L locus, in some embodiments using commercially available reagents. By co-transfecting plasmids containing (i) individual guide RNAs upstream from the DNMT3L gene, and (ii) a “dead” Cas9 cDNA engineered to organize a transcription complex at that site in the genome, along with a second plasmid designed to express an appropriate transcriptional complex, the present disclosure demonstrates that these reagents can strongly activate DNMT3L expression in K562 human erythroleukemia cells, as shown in FIG. 1. Western blot showing levels of DNMT3L protein (black) or ACTIN (red) in K562 cells treated for 1 or 2 days (D1 or D2) with 0 or 10 nM Romidepsin (R10, left-most lanes). To the right, levels of DNMT3L protein are shown 1, 2, or 3 days after electroporation of plasmids containing dead Cas9 and one of three guide RNAs targeted upstream from the endogenous DNMT3A gene (g1, g2, and g3), or a scrambled guide RNA (scr). All were co-transfected with a second plasmid containing the transcriptional activator complex. Note the very high levels of activation of DNMT3L protein with guides g2 and g3 at days 2 and 3. This level of expression persisted until at least 7 days after transfection (data not shown).

(iv) Use of novel methods is explored herein to deliver the CRISPR/a plasmids to AML cells in vitro and in vivo, using both liposomal and/or nanoparticle based methods to deliver these plasmids to AML cells. These particles could be modified to deliver their payloads specifically to AML cells defined by cell surface proteins that are specific for AML.

(v) Use of modified mRNAs is also explored to express DNMT3L in AML cells. These RNAs, which contain specific modifications designed to reduce their immunogenicity, and increase their stability, may be delivered by nanoparticle-based systems that employ short peptides, dendrimers, or other strategies (depending upon the embodiment) to deliver payloads to AML cells.

### Example 2: Rapid and Accurate Remethylation of Dnmt3a Deficient Hematopoietic Cells with Restoration of Dnmt3a Activity

This Example describes a novel strategy for increasing the activity of DNMT3A in hematopoietic cells (including AML cells) as necessitated by dominant negative or loss-of-function DNMT3A mutations.

Heterozygous loss-of-function mutations in the DNMT3A gene are the most common cause of clonal hematopoiesis, and among the most common initiating events for Acute Myeloid Leukemia (AML). A reduction of DNMT3A activity causes a canonical, focal hypomethylation phenotype in hematopoietic cells, which is associated with immortalization of hematopoietic stem cells, and age-dependent myeloid skewing. Over a period of several months, the methylation defect can be partially reversed with restoration of physiologic levels of DNMT3A expression. Herein is characterized the DNA methylation phenotypes of bone marrow cells from mice with hematopoietic cell deficiency of Dnmt3a, Dnmt3b (or both enzymes), or expressing the dominant negative Dnmt3aR878H mutation (R882H in humans; the most common mutation found in AML patients). Using these bone marrow cells as substrates, the patterns and completeness of DNA remethylation after “adding back” supraphysiologic levels of wild type DNMT3A1, DNMT3B1, DNMT3B3 (an inactive splice isoform of DNMT3B), or DNMT3L (a catalytically inactive “chaperone” for DNMT3A and DNMT3B in early embryogenesis) were defined. High level expression of DNMT3A for two weeks can accurately reverse the hypomethylation phenotype of Dnmt3a deficient cells, or cells expressing the R878H mutation.

Remarkably, overexpression of DNMT3L (which is not expressed in AML cells) can likewise correct the hypomethylation phenotype of Dnmt3aR878H/+ bone marrow cells ex vivo and in vivo, by augmenting the activity of WT DNMT3A encoded by the residual WT allele. These data suggest that reactivation of DNMT3L represents a novel approach for restoring DNMT3A activity in AML initiated by DNMT3A mutations.

A finalized publication contributing to the present disclosure entitled “Rapid and accurate remethylation of DNA in Dnmt3a-deficient hematopoietic cells with restoration of DNMT3A activity” to Li et al. (2024), Sci Adv., v.10(5), and all associated supplemental materials are herein incorporated by reference as support of the present disclosure.

Herein is characterized the DNA methylation phenotypes of bone marrow cells from mice with hematopoietic deficiency of Dnmt3a or Dnmt3b (or both enzymes) or expressing the dominant-negative Dnmt3a^{R878H} mutation [R882H in humans; the most common DNMT3A mutation found in acute myeloid leukemia (AML)]. Using these cells as substrates, DNA remethylation was defined after overexpressing wild-type (WT) DNMT3A1, DNMT3B1, DNMT3B3 (an inactive splice isoform of DNMT3B), or DNMT3L (a catalytically inactive “chaperone” for DNMT3A and DNMT3B in early embryogenesis). Overexpression of DNMT3A for 2 weeks reverses the hypomethylation phenotype of Dnmt3a-deficient cells or cells expressing the R878H mutation. Overexpression of DNMT3L (which is minimally expressed in AML cells) also corrects the hypomethylation phenotype of Dnmt3aR878H/+ marrow, by augmenting the activity of WT DNMT3A encoded by the residual WT allele. DNMT3L reactivation may represent a previously unidentified approach for restoring DNMT3A activity in hematopoietic cells with reduced DNMT3A function.

### Introduction

DNMT3A and DNMT3B are the dominant mammalian de novo DNA methyltransferases, required for generating CpG methylation patterns in early embryogenesis, and are essential for tissue specification and development. In early embryogenesis, the methyltransferase activity of these proteins is greatly augmented by DNMT3L, a highly related chaperone protein that has no DNA methyltransferase activity per se; however, it forms heterotetramers with DNMT3A or DNMT3B that greatly increase their DNA methyltransferase activities. The DNMT3L gene is silenced by epigenetic mechanisms in adult tissues and is minimally expressed in normal or malignant hematopoietic cells. However, studies in tissue culture cells have suggested that DNMT3L can be reactivated by hypomethylating agents (HMAs; like 5-azacytidine) and/or histone deacetylase (HDAC) inhibitors, which can synergize to augment its expression. Further, when DNMT3L is co-expressed with DNMT3A (but not DNMT3B) in human 293 tissue culture cells, it greatly increases the activity of DNMT3A, increasing methylation at many sites throughout the genome.

In normal mouse and human hematopoietic cells, the active isoforms of DNMT3A (DNMT3A1 and DNMT3A2) are highly expressed during early development and then gradually silenced during terminal differentiation. Dnmt3a is not absolutely required for hematopoiesis in mice: A complete loss of Dnmt3a in the germline yields a phenotypically normal hematopoietic compartment that can be harvested at 2 to 3 weeks of age and transplanted into secondary recipients. These bone marrow cells have a focal, canonical DNA hypomethylation phenotype, but they can reconstitute all hematopoietic lineages in lethally irradiated recipients. Hematopoietic stem cells completely deficient for Dnmt3a appear to be immortalized with serial transplantation, and, over time, their progeny develop myeloid skewing and an increased risk of the development of hematopoietic malignancies.

Likewise, Dnmt3b-deficient bone marrow cells can reconstitute long-term hematopoiesis, but they have reduced B cells, mimicking what is seen in human patients with the “immunodeficiency, centromeric instability, and facial dysmorphism syndrome, type I” [Online Mendelian Inheritance in Man (OMIM), no. 242860], often caused by loss-of-function mutations in the DNMT3B gene. In adult hematopoietic cells and acute myeloid leukemia (AML) cells, the DNMT3B gene is expressed, but the dominant isoform is DNMT3B3, which does not contain part of the DNA methyltransferase domain. Although DNMT3B3 is inactive as a methyltransferase, recent studies have demonstrated its ability to interact with DNMT3A to augment its activity, signifying that it may have a “chaperone” function for DNMT3A in adult hematopoietic cells, akin to that of DNMT3L in embryonic cells. The DNA methylation phenotype in Dnmt3b-deficient bone marrow cells was inferred in a previous study, which measured methylation phenotypes in hematopoietic stem/progenitor cells (HSPCs) from Dnmt3a or Dnmt3a×Dnmt3b-deficient mice, but not from Dnmt3b-deficient mice per se. The study suggested that the two enzymes may have unique methylation target sites in the genome, but this has yet to be directly verified in humans or in mice.

While DNMT3B mutations are rarely detected in hematopoietic malignancies, DNMT3A mutations are very common in patients with clonal hematopoiesis and/or myeloid malignancies, and also cause the DNMT3A overgrowth syndrome (DOS). Nearly all DNMT3A mutations in these diseases appear to cause loss-of-function, resulting in focal, canonical DNA hypomethylation phenotypes that vary in severity. Deletions and premature stop codons (i.e., causing haploinsufficiency) can cause mild hypomethylation phenotypes that are very similar to that of many missense mutations, indicating that many of these also cause loss of function. Mutations at amino acid R882 in humans (R878 in mice) cause a more severe hypomethylation phenotype, reflecting the dominant-negative activity of these mutations; they directly reduce methyltransferase activity by ˜80%, and the mutant protein also preferentially interacts with wild-type (WT) DNMT3A, trapping it in inactive heterodimeric complexes. Despite the methylation consequences of these mutations, patients with DOS and patients with clonal hematopoiesis can live for many years (or even decades) with essentially normal blood counts, so the effects on blood cell development are relatively small and slow to develop. Because nearly all DNMT3A mutations are heterozygous, a residual WT allele is usually present and expressed, providing a source of WT DNMT3A to target in order to overcome the loss-of-function or dominant-negative effects of mutations if its activity could be increased.

In this study, whole-genome bisulfite sequencing (WGBS) was used to define the DNA methylation phenotypes of the bone marrow cells of adult mice that had undergone somatic inactivation of Dnmt3a or Dnmt3b (or both enzymes) at embryonic days 9 to 10, using Vav1-Cre to induce near-complete floxing of these alleles in hematopoietic cells. Likewise, the methylation phenotype of Dnmt3aR878H/+×Vav1-Cre bone marrow cells was defined, which is intermediate in severity between haploinsufficiency and complete loss of Dnmt3a expression. In all these studies, bone marrow cells were derived directly from unmanipulated mice, eliminating potential methylation phenotypes caused by the stress of transplantation (which increases HSPC cycling and the expression of Dnmt3a). It was previously shown that the differentially methylated regions (DMRs) in Dnmt3a-deficient HSPCs, progenitor populations, mature B cells, and mature myeloid cells are nearly identical, strongly suggesting that Dnmt3a must perform de novo methylation predominantly in early stem/progenitor cells; in some embodiments, this methylation phenotype is maintained in differentiated progeny by Dnmt1. These observations make it possible to examine the consequences of Dnmt3a mutations in unfractionated whole-bone marrow samples, because all of the purified populations have similar methylation phenotypes at regions where DNMT3A acts.

Last, the effects of retroviral overexpression of DNMT3A1, DNMT3B1, DNMT3B3, and DNMTL were explored on the DNA methylation phenotypes of bone marrow cells deficient for Dnmt3a, Dnmt3b, or both enzymes. Hypomethylation phenotypes were corrected within weeks by overexpressing DNMT3A1 or DNMT3B1. Methylation can also be completely and rapidly corrected in cells expressing a heterozygous Dnmt3aR878H/+ mutation by overexpressing DNMT3L, which may act to increase the activity of WT DNMT3A protein encoded by the residual WT allele. These data clarify the contributions of DNMT3A and DNMT3B to the methylation patterns of adult hematopoietic cells and designate a strategy for increasing the activity of DNMT3A in AML cells initiated by DNMT3A mutations.

### Results

### Focal, Canonical Hypomethylation Phenotypes in the Bone Marrow Cells of Dnmt3a- or Dnmt3b-Deficient Mice

Previous studies of the methylation patterns of Dnmt3a-deficient hematopoietic cells have predominantly used transplanted cells from mice with germline mutations or conditional knockout mice that have been serially transplanted. To better understand the consequences of the loss of DNA methyltransferases in unmanipulated hematopoietic cells, the Vav1-Cre transgene was used to induce floxing at embryonic days 9 to 10 in mice with Dnmt3aflox/flox, Dnmt3bflox/flox, or Dnmt3aflox/flox×Dnmt3bflox/flox alleles.

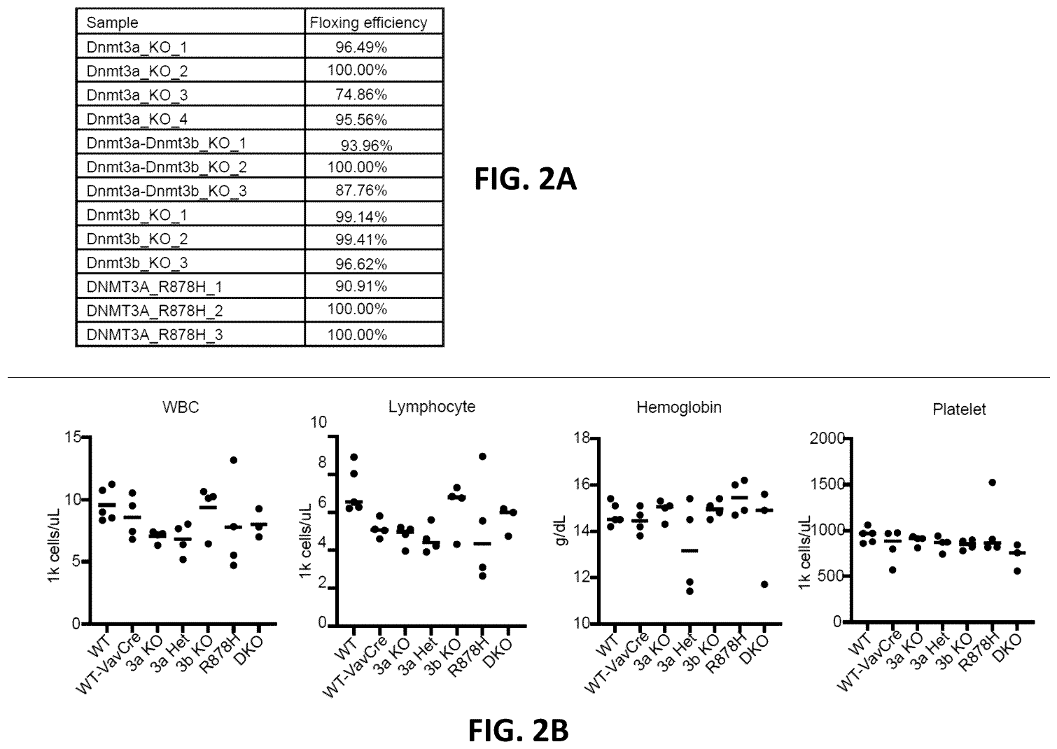

Floxing efficiency was determined in the bone marrow samples of every tested mouse using sequence coverage of the floxed (i.e., deleted) regions of each gene, measured in the whole-genome bisulfite data (FIG. 2A); the mean value for all mice was 96%. Mice from these crosses were born at the expected Mendelian frequencies and had no overt abnormalities in growth or development. At 6 to 8 weeks of age, their blood counts were not significantly different from those in WT mice (FIG. 2B). Whole-bone marrow cells were harvested from mice at 6 to 12 weeks of age for DNA methylation studies. WGBS was performed on all samples to define the CpG methylation phenotypes across the genome. For all WGBS samples combined in all studies, a mean genome coverage of 18.6× (minimum coverage, 13.1×) was achieved. DMRs were first defined by comparing four bone marrow samples from Dnmt3aflox/flox×Vav1-Cre mice (henceforth “3a KO,” one male and three females) to nine WT bone marrow samples (“WT,” six males and three females) using previously published methods, to identity Dnmt3a-dependent methylation changes (FIGS. 3A and 3B). DMRs were defined as having at least 10 CpGs, a mean methylation difference between two groups of 0.2 and a false discovery rate (FDR) of <0.05. Contiguous DMRs within 50 base pairs (bp) of each other were combined. 10,724 DMRs (FIG. 3A) were identified in the 3a KO mice; there were no differences based on the sex of the mice. The average width of these DMRs was 746 bp, and the mean number of CpGs per DMR was 19.5. In total, these DMRs encompassed ˜8 Mb of DNA, accounting for about 0.3% of the mouse genome. A total of 10,714 of the 10,724 DMRs (˜99.9%) were hypomethylated in the 3a KO bone marrow samples. Also, the mean methylation values were “passively” plotted for the same DMRs in Dnmt3bflox/flox×Vav1-cre bone marrow samples (“3b KO”), samples from mice deficient for both enzymes (“DKO”), or samples from Dnmt3aR878H/+×Vav1-Cre mice (“R878H”). The 3b KO and R878H samples had more subtle methylation changes at these 10,724 DMRs, and the DKO samples had more pronounced reductions in methylation. The aggregated mean methylation values for the 3a KO DMRs are shown in a summary “canyon plot” in FIG. 3B, which quantifies the average differences in methylation for each genotype at the 3a KO DMRs.

A comparison of the same nine WT samples and three 3b KO samples (all males) yielded 2012 DMRs (FIGS. 3C and 3D), with a mean size of 597 bp and an average of 17 CpGs per DMR, encompassing ˜1.2 Mb (0.045% of the mouse genome). A total of 2009 of the 2012 DMRs (˜99.9%) were hypomethylated. Passive plotting of the mean methylation values of the 3b KO DMRs was performed for the 3a KO samples, the DKO samples, and the R878H samples (FIG. 3C). In most of these regions, the 3a KO samples were likewise hypomethylated, suggesting that Dnmt3a and Dnmt3b may act at many of the same genomic regions. However, some regions in the 3b KO samples were more hypomethylated than the 3a KOs; manual review of these regions revealed that the vast majority also had reduced methylation in the 3a KO samples (only two DMRs appeared to be 3b KO specific). The mean methylation values of 3b KO DMRs were further reduced in the DKO samples, again showing that the DNMT3A and DNMT3B may be acting synergistically at many sites. The R878H methylation values at 3b KO DMRs were similar to those of the 3a KO samples. Comparative quantification of the aggregated mean methylation values of these DMRs for all genotypes is shown in the canyon plot and is consistent with the observations in the heatmap (FIG. 3D).

DMRs were next defined in the DKO bone marrow samples (three males) versus the same nine WT samples. 23,411 DMRs (FIGS. 3E and 3F) were detected, of which 23,408 of the 23,411 DMRs (99.99%) were hypomethylated. These DMRs span 21.4 Mb of DNA in total (˜0.8% of the mouse genome). The mean size of these DMRs was 916 bp, with an average of 23 CpGs per DMR. Passive plotting of these DMRs with data from the other genotypes revealed that the DKO DMRs were the most hypomethylated and that all genotypes have reduced DNA methylation at these sites in the genome (FIG. 3F).

The phenotypes of Dnmt3aR878H/+×Vav1-Cre mice were also evaluated using an established floxed allele. Using RNA sequencing (RNA-seq) data from the bone marrows of WT and R878H mice, the mean floxing efficiency was determined in these mice to be >96%. These mice had no gross phenotypic abnormalities at 6 to 12 weeks of age (i.e., no overgrowth or obesity) and had essentially normal complete blood counts (FIG. 2B). Bone marrow cells were harvested from six Dnmt3aR878H/+ mice (R878H, four males and two females) for WGBS. By comparing these data to WT bone marrow samples, 4453 DMRs were identified in the R878H samples (FIGS. 3G and 3H, and table S4), of which 4450 were hypomethylated (99.9%). The average size of R878H DMRs was 672 bp, and each contained a mean of 20 CpGs; these DMRs encompassed ˜3 Mb of DNA (˜0.1% of the mouse genome). Passive plotting of these DMRs for the other genotypes revealed that all had some reduction of methylation at virtually all sites; this reduction was uniformly more pronounced for the 3a KO and DKO samples. No differences could be ascribed to the sex of the animals.

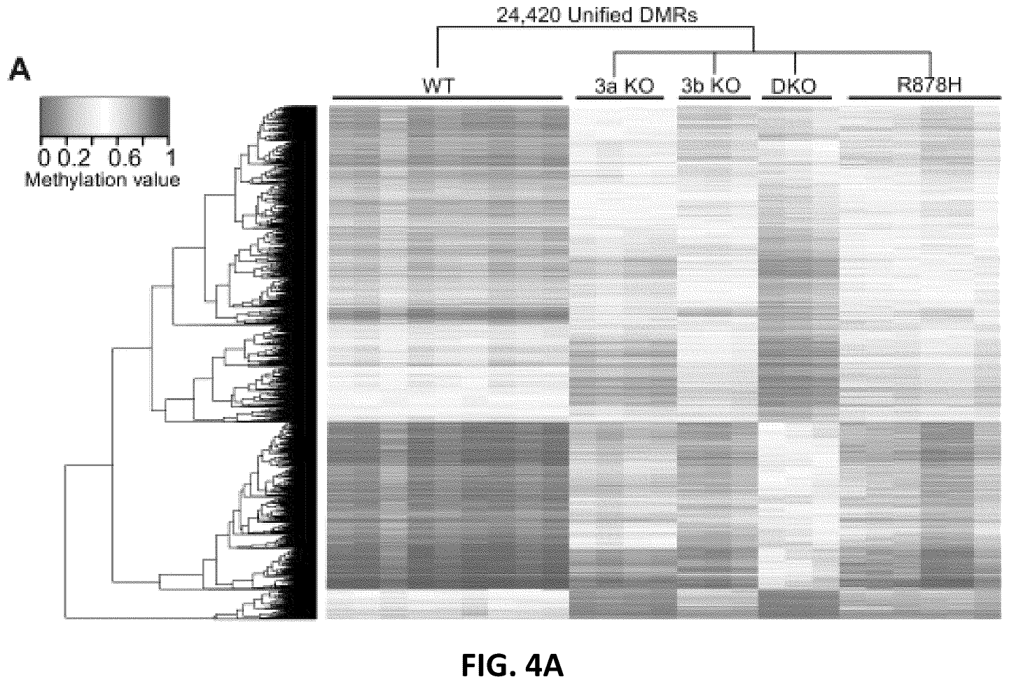

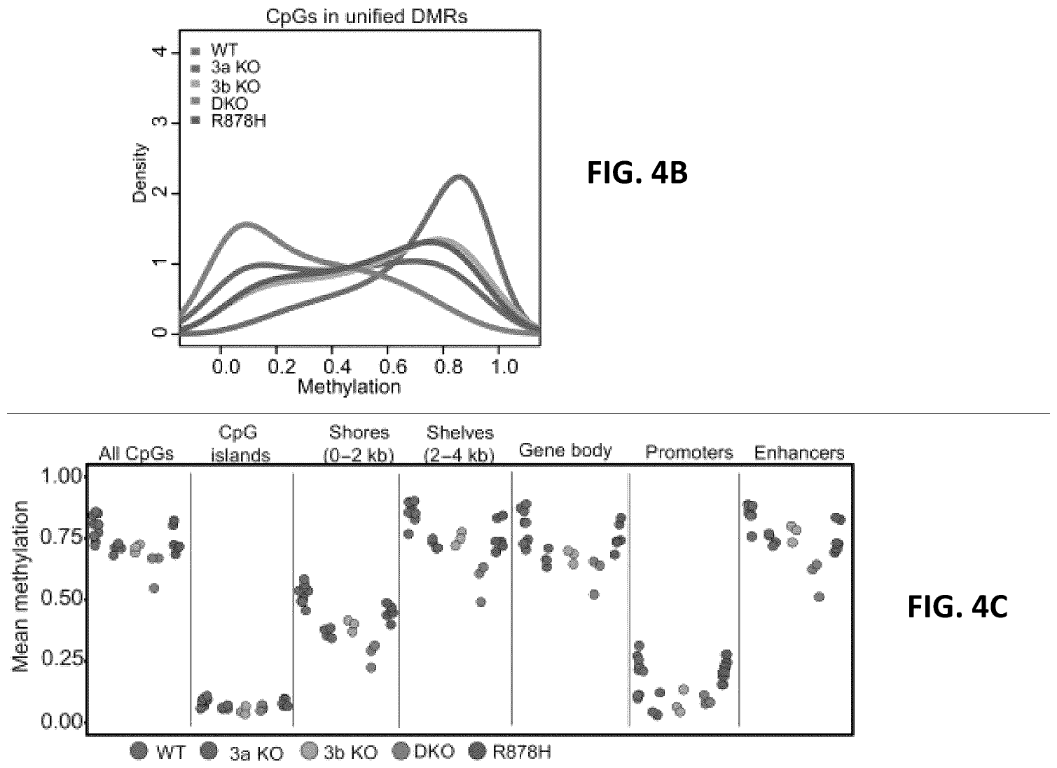

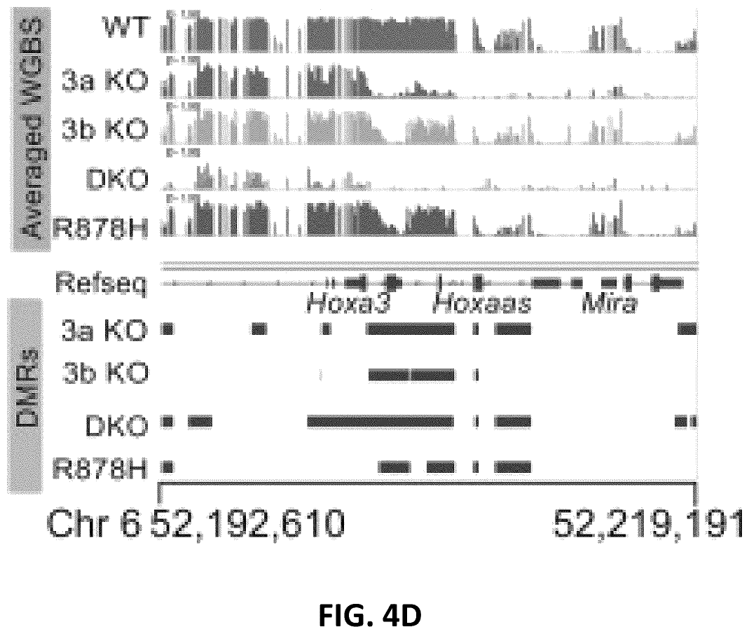

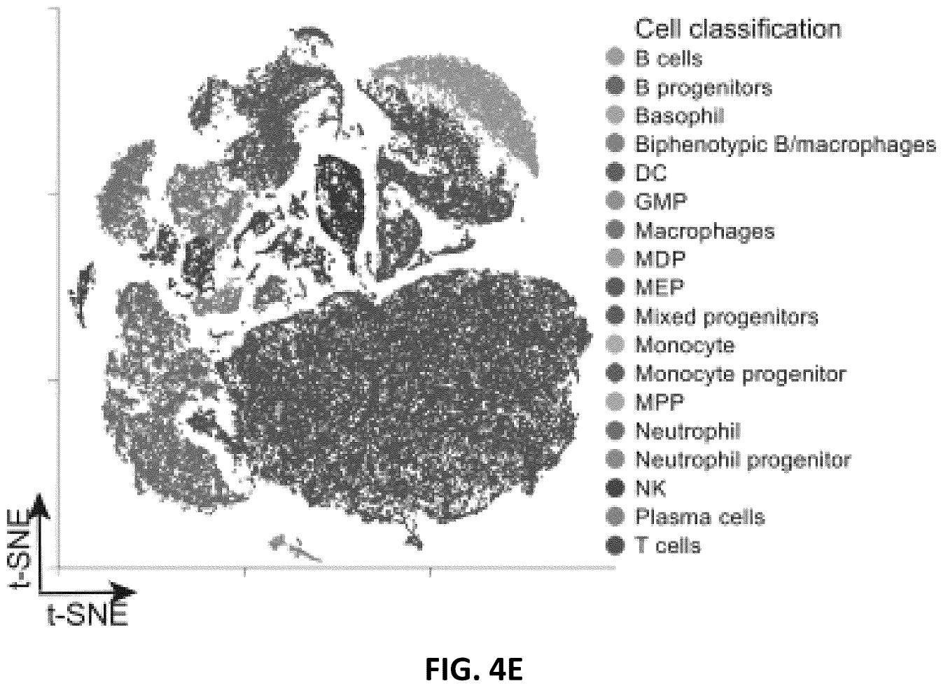

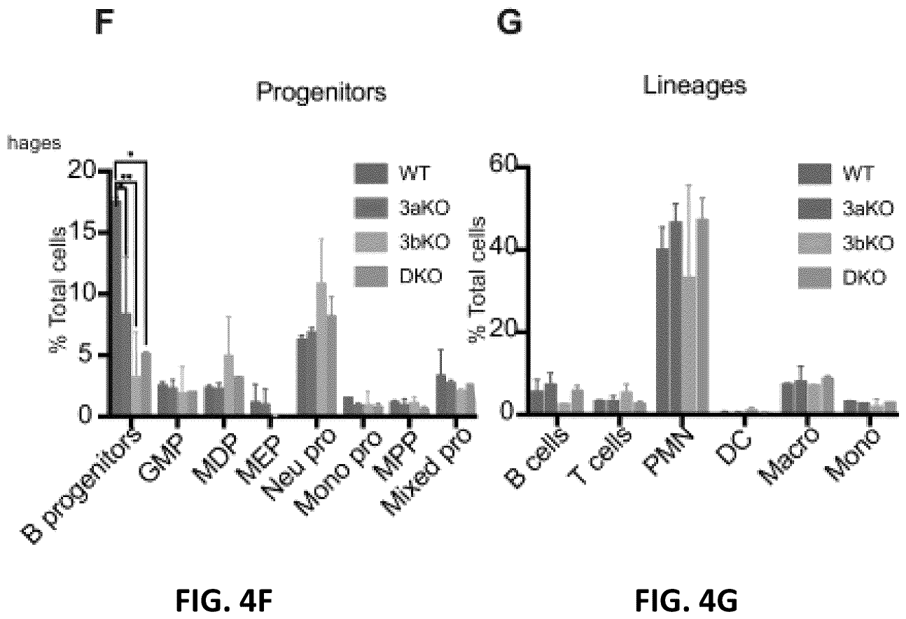

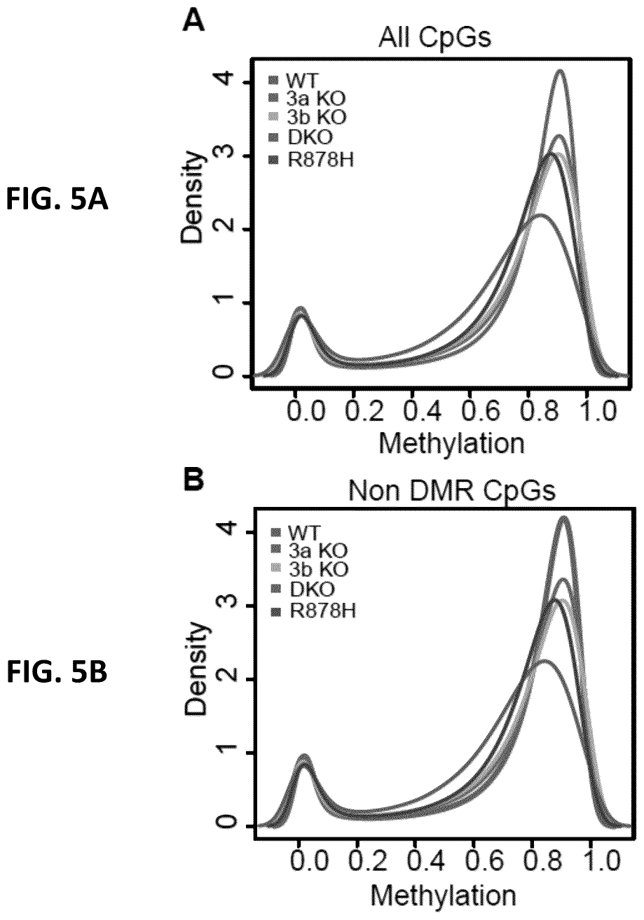

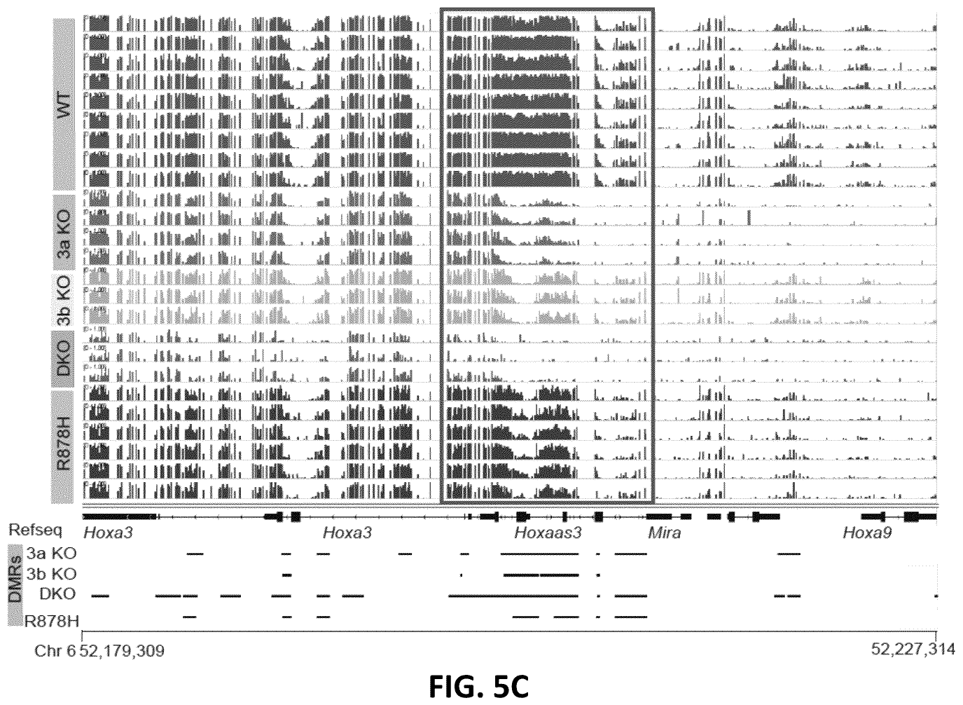

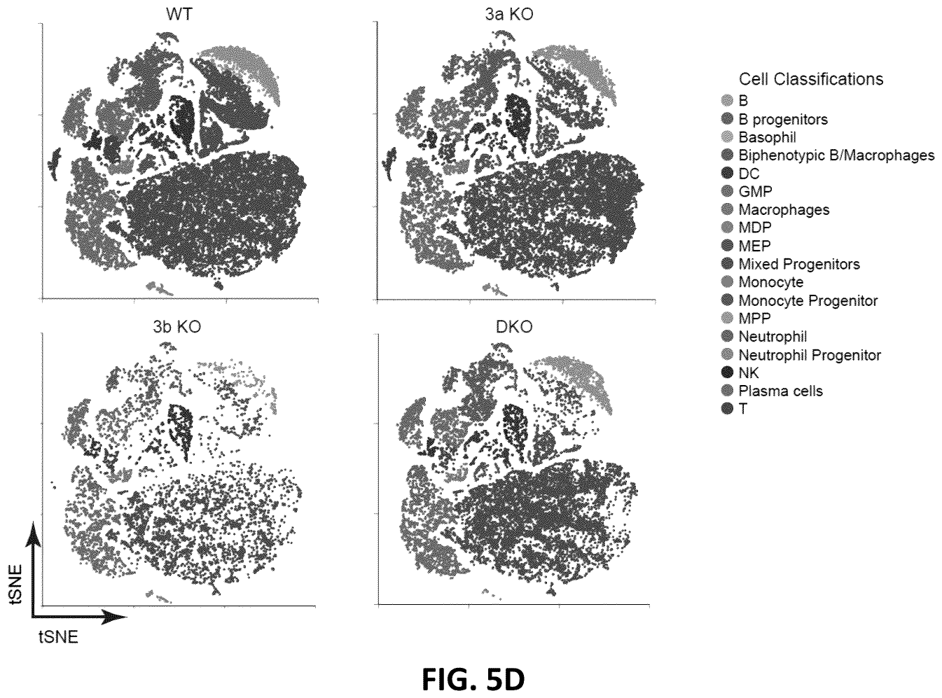

To evaluate the global methylation defects caused by loss of function of Dnmt3a and Dnmt3b, the union of all DMRs was taken from the above comparisons, merging overlaps to create a “unified set” of 24,420 DMRs, plotted for all genotypes in FIG. 4A (table S5). No consistent differences could be ascribed to the sexes of the mice. As expected, DKO samples had the lowest methylation levels at nearly all DMRs. 3a KO methylation values were the next most hypomethylated, thus pointing out the important role of Dnmt3a in defining these DMRs. To extend these findings to all CpGs (i.e., not just DMRs), a global analysis of CpG methylation values was first performed for all samples in the methylation density plot shown in FIG. 4B. The relative methylation values for all CpGs were WT>3b KO≥R878H>3a KO>DKO. A plot showing CpG methylation values in annotated regions of the genome for all samples is shown in FIG. 4C (also see FIGS. 5A and 5B); because DMRs encompass less than 1% of the genome, the overall differences from WT samples were relatively small. All of the differences from WT samples were statistically significant. The most notable changes in methylation were found in shores, shelves, gene bodies, and enhancers, which are much more methylated than CpG islands and promoters. In all regions, the DKO samples had the most notable reductions in methylation, and the R878H samples tended to have milder changes. To directly visualize the differential methylation phenotypes for all genotypes at a typical, informative DMR (in the HoxA gene cluster), an Integrative Genomics Viewer (IGV) view of the methylation values for all CpGs in this region is shown in FIG. 4D. The size and shape of the DMRs in this region reflect the depth and width of DMRs genome-wide for these genotypes, providing context for the average values shown for methylation in the heatmaps. An IGV view of each sample plotted individually for this locus is shown in FIG. 5C (highlighted by red box), demonstrating the reproducibility of this phenotype across many WGBS samples from different mice.

Last, to confirm that the altered methylation phenotypes are due to the cell-intrinsic loss of DNA methyltransferases per se (and not to shifts in hematopoietic cell populations with altered DNA methylation patterns), the 10× Genomics Chromium platform was used to perform single-cell RNA-seq (scRNA-seq) on whole-bone marrow cells derived from two independent mice each (6 to 12 weeks of age) for WT, 3a KO, 3b KO, and DKO mice. Cell identities were inferred on the basis of the Haemopedia Database and graphic-based manual review (FIG. 4E and FIG. 5D). The numbers of early B cells were significantly reduced in 3a KO, 3b KO, and DKO marrows compared to those in WT, with 3b KOs showing the most reduction (FIG. 4F). The mature B cell population in 3b KO cells was also reduced, but the change was not statistically significant (FIG. 4G). The sizes of all other populations were not statistically different from WT marrow samples. Collectively, these data suggest that the genotype-specific change in DNA methylation is not due to markedly altered marrow populations but rather due to the loss-of-function mutations themselves.

### Correction of Dnmt3a KO DMRs by Addback of DNA Methyltransferases

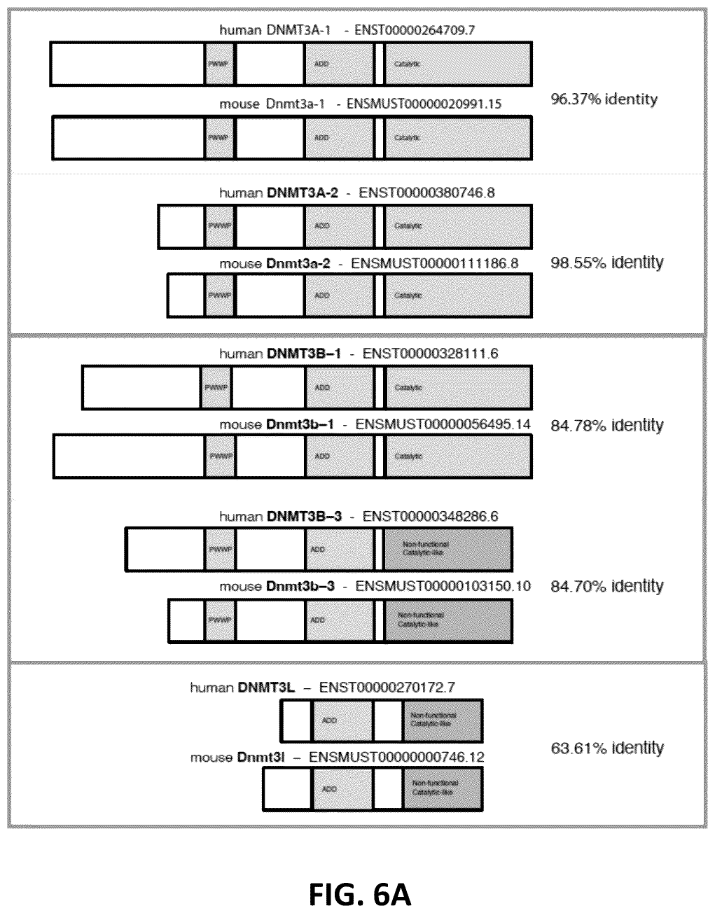

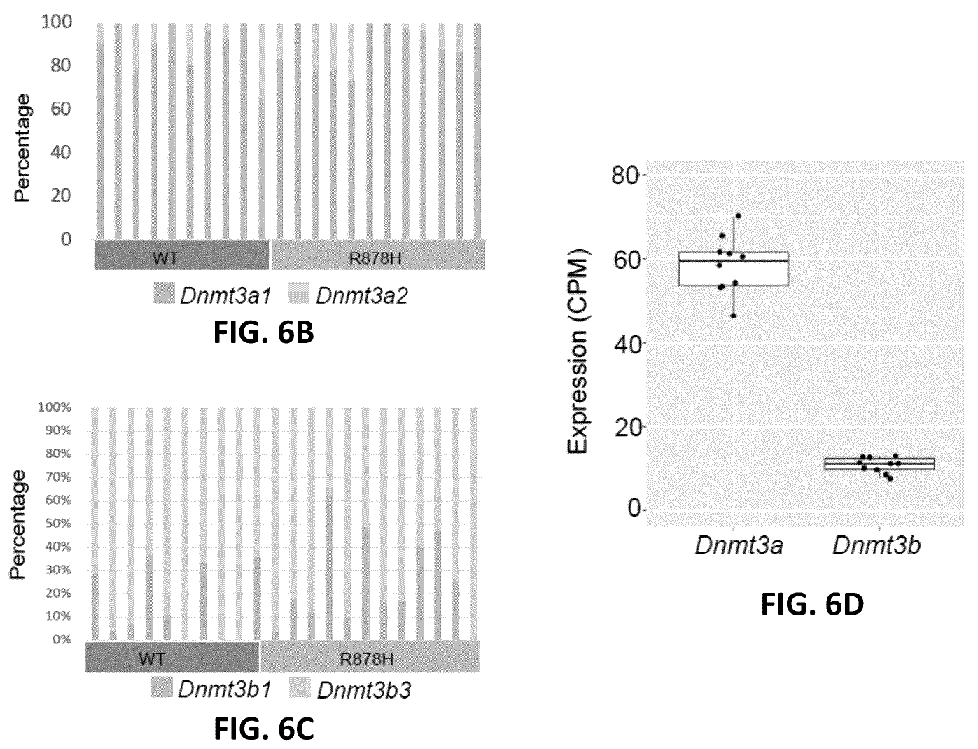

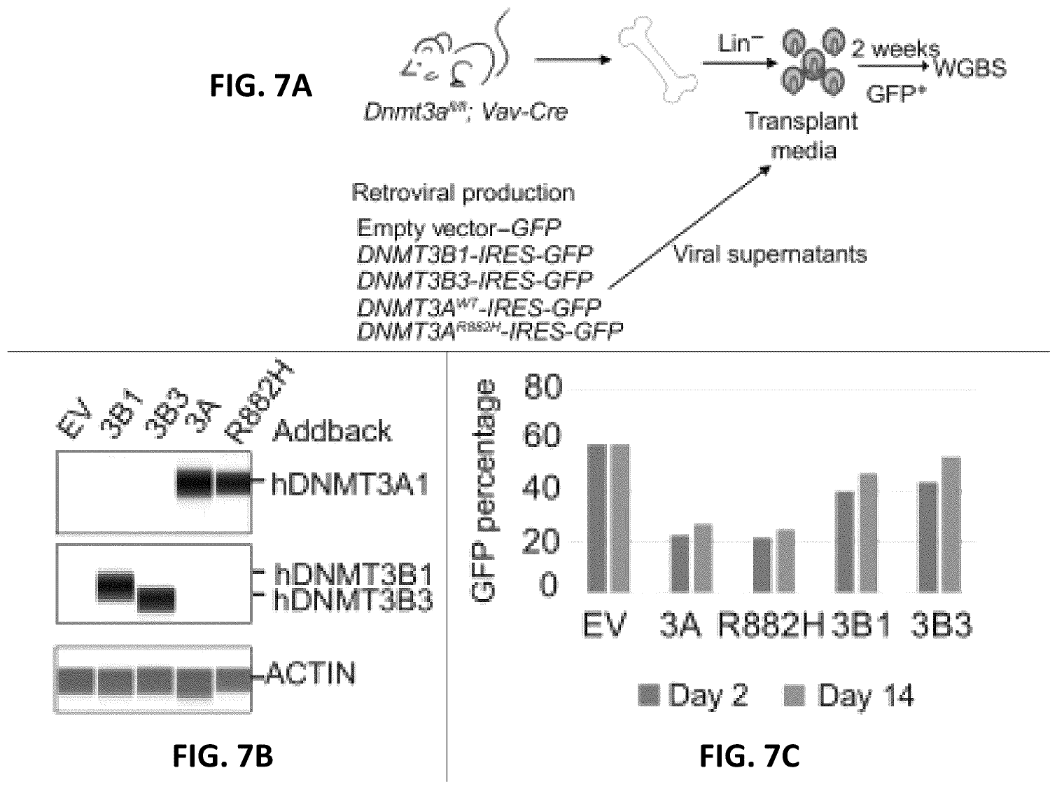

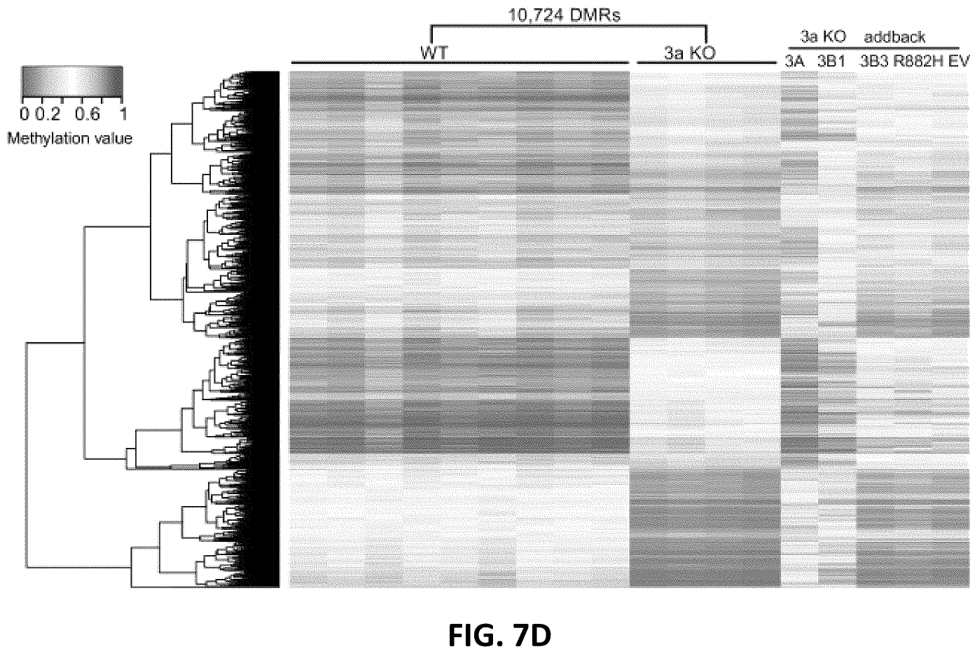

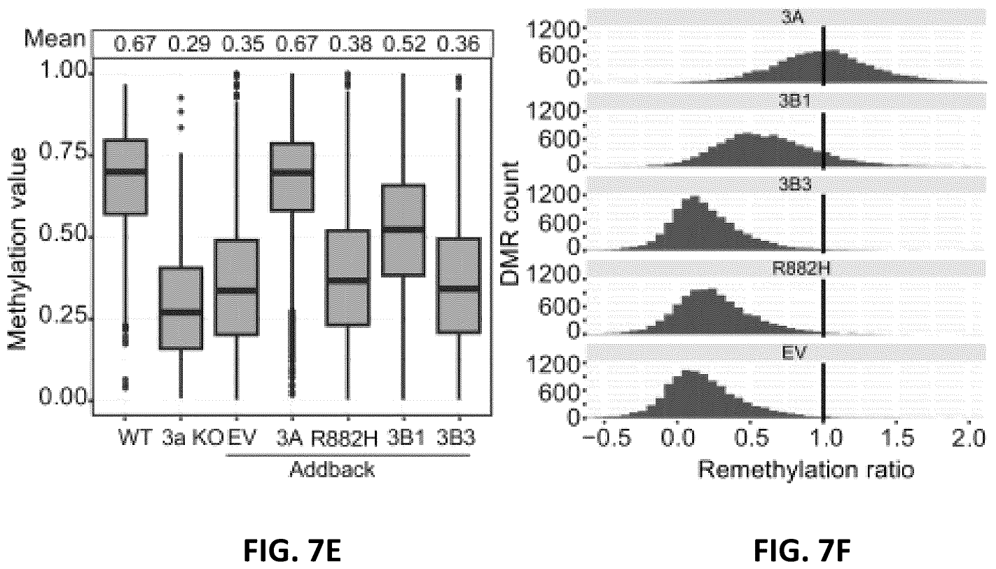

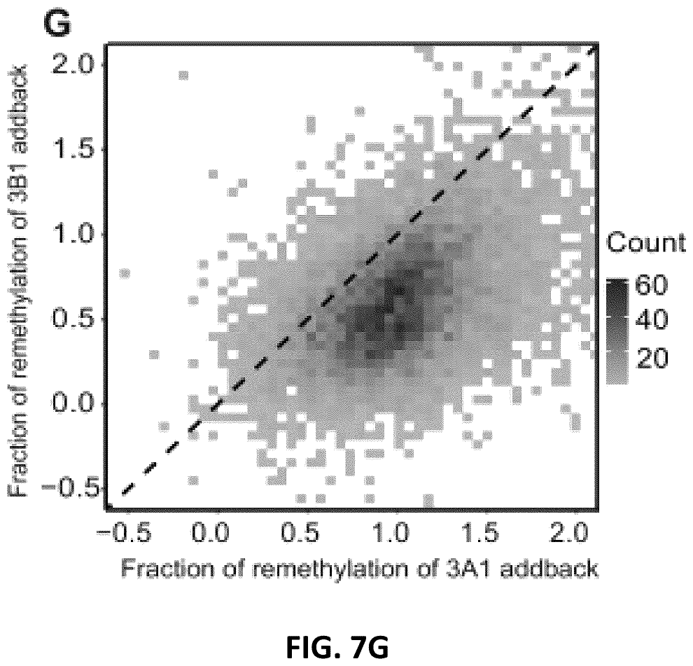

To determine whether the hypomethylation phenotype of Dnmt3a-deficient mouse bone marrow could be corrected with a genetic addback approach, retroviral vectors were created to express full-length human DNMT3A1, DNMT3B1, or DNMT3B3 cDNAs in mouse hematopoietic cells. Human cDNAs were used because these genes are highly conserved between mice and humans (FIG. 6A) and because it has been previously shown that a human DNMT3A1 transgene can completely correct the focal hypomethylation phenotype in Dnmt3a-deficient mouse hematopoietic cells; a further goal is to use proven cDNAs to restore DNMT3A function in human cells. To define the most common splicing isoforms expressed in mouse bone marrow cells, bulk RNA-seq data was analyzed from WT versus R878H mice, which revealed that the dominant isoform of Dnmt3a is Dnmt3a1 [88.6% of transcripts in WT (n=10; range, 63 to 100%) and 88.5% in R878H (n=12; range, 73 to 100%), P=1.0], and the dominant isoform of Dnmt3b is Dnmt3b3 [78.2% of transcripts in WT (n=10; range, 63 to 100%) and 78.1% in R878H (n=12; range, 37 to 100%), P=1.0] (FIG. 7A and FIGS. 6B and 6C). Transduced cells were harvested 2 days after back-to-back daily transductions to define the fractions of cells that were green fluorescent protein (GFP)+; purified GFP+ cells were used to perform quantitative Western blots (using the “ProteinSimple” platform) to ascertain the level of expression of proteins from each transduced retrovirus; no endogenous DNMT3A was detected in the 3a KO cells, but this protein was expressed in cells transduced either with WT DNMT3A1 or the same cDNA with the R882H mutation (which reduces methyltransferase activity by ˜80%) (FIGS. 7B and 7C); compared to endogenous DNMT3B3 levels detected in the empty vector (EV)-transduced cells on same Western blot (FIG. 7B), retroviral DNMT3B1 levels were increased ˜17-fold, and DNMT3B3 levels were increased ˜16-fold. Transduced cells were then placed in “transplant media” [RPMI 1640 with 15% fetal bovine serum (FBS) and murine interleukin-3 (IL-3), stem cell factor (SCF), FMS-like tyrosine kinase 3 ligand (FLT3L), and thrombopoietin (TPO)] for 2 weeks, and GFP percentages were determined; fractions of GFP+ cells were minimally altered from baseline values (FIG. 7C), suggesting that overexpression of these proteins does not select for or against expressing cells over this time frame. DNA methylation was assessed by performing WGBS from DNA harvested from purified GFP+ cells at day 14 and is displayed in heatmap form in FIG. 7D. DMRs previously defined for WT versus 3a KO bone marrow samples were used to calibrate DNA methylation changes caused by Dnmt3a deficiency, and methylation values for each of the “addback” samples were plotted passively at these genomic locations. DNMT3A1 addback to 3a KO samples remethylated nearly all of the 3a KO DMRs. Overexpression of DNMT3B1 partially remethylated the 3a KO DMRs. Overexpression of DNMT3B3, DNMT3AR882H, and the MSCV “EV” minimally altered methylation of the 3a KO DMRs, as expected. The data presented in the heatmap are quantified (FIG. 7E to 7G) to reveal that nearly all of the hypomethylated 3a DMRs are remethylated by DNMT3A1 overexpression for 2 weeks, without evidence for excess methylation at these sites. In contrast, DNMT3B1 overexpression remethylated a large fraction of the 3a KO DMRs but less efficiently than DNMT3A1 (FIGS. 7F and 7G). Overexpression of DNMT3B3 or DNMT3AR882H had minimal effects on remethylation, similar to that of the empty retroviral vector.

### DNMT3B3 Facilitates DNA Remethylation in a DNMT3A-Dependent Manner

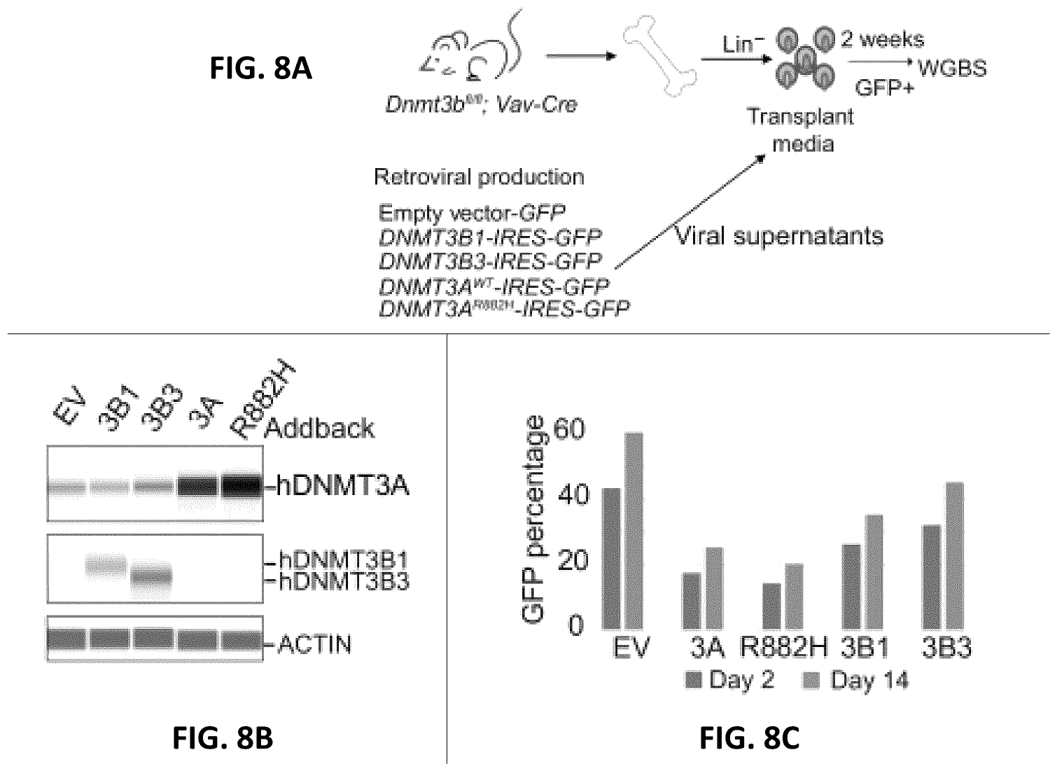

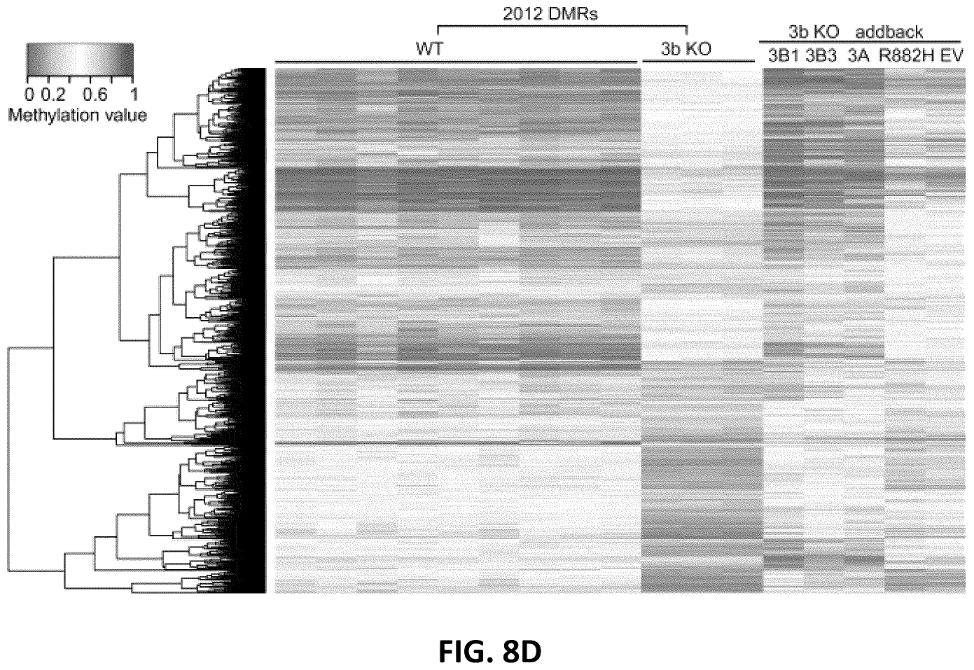

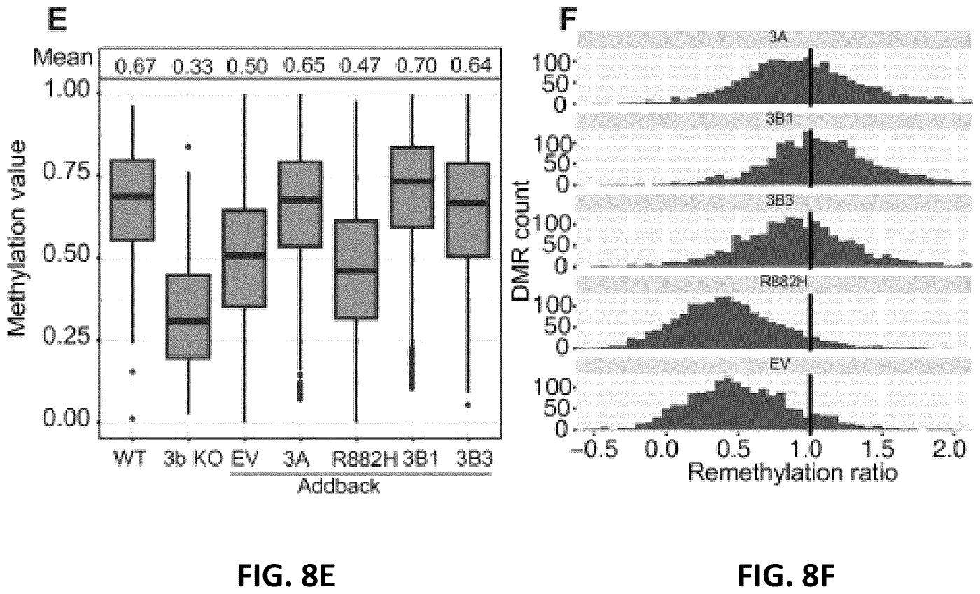

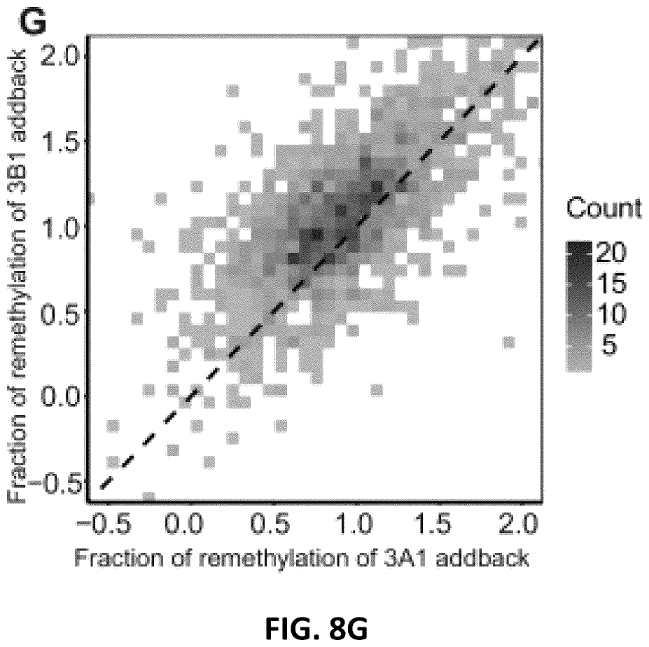

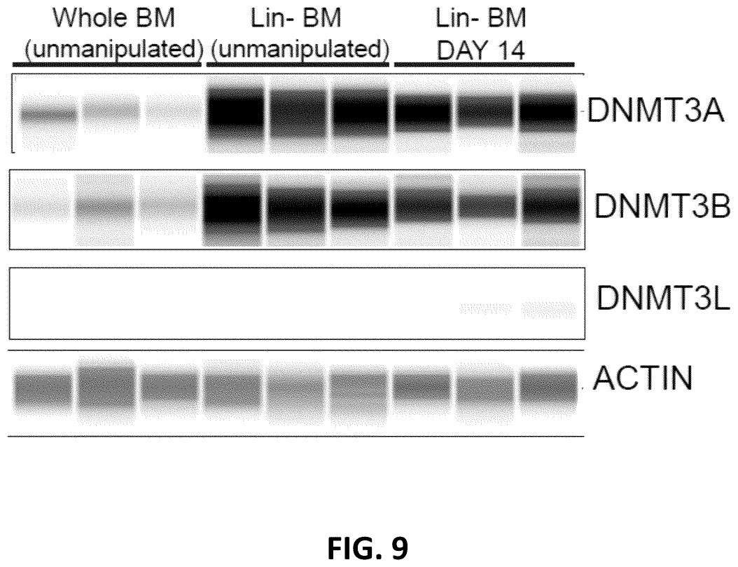

RNA-seq analysis of adult mouse bone marrow cells revealed that the majority of Dnmt3b transcripts are of the Dnmt3b3 isoform, which does not contain a large portion of the methyltransferase domain (FIG. 6C). To further define the functions of DNMT3B1 and DNMT3B3 in hematopoietic cells, an ex vivo addback experiment was conducted using Dnmt3b-deficient mouse bone marrow cells, using an experimental strategy identical to the one shown in FIG. 7(A-G) (FIG. 8A). Overexpression of appropriately sized DNMT3B proteins was confirmed by Western blotting of samples obtained 2 days after transduction (FIG. 8B); in these samples, the endogenous level DNMT3A protein expression and determined that WT DNMT3A1 was overexpressed 2.5-fold compared to the level detected in EV-transduced cells and that DNMT3A1 R882H was overexpressed ˜3.7-fold (the smaller apparent fold change for DNMT3A [˜3× versus ˜16× for 3B] reflects the 5.4-fold higher background level of endogenous Dnmt3a mRNA in mouse bone marrow cells, which reduces the apparent fold change that can be achieved for 7A; FIG. 6D). Flow cytometry revealed that the fraction of GFP+ cells was similar for all constructs at days 2 and 14 (FIG. 8C). DNA was harvested from GFP+ cells for all transductions on day 14 and subjected to WGBS (FIG. 8D-8G). Methylation values of DMRs defined by comparing WT versus 3b KO bone marrow cells (n=2012) were used to define the impact of the addbacks, which were passively plotted for the same DMRs. As expected, DNMT3B1 restored the methylation values of the 3b KO DMRs to levels that were nearly equivalent to WT samples (FIG. 8D, 8E, 8F). Likewise, DNMT3A1 restored methylation at the 3b KO DMRs to levels that were nearly equivalent to WT or DNMT3B1 addback cells, indicating that DNMT3A1 and DNMT3B1 can remethylate the same regions of the genome (FIG. 8G). DNMT3B3 overexpression likewise induced remethylation of the 3b KO DMRs to near WT levels, such that DNMT3B3 may be augmenting the activity of DNMT3A to cause remethylation at these sites; DNMT3B3 was shown to be completely inactive in addback experiments with 3a KO cells (see above) or DKO cells (see below). The EV and DNMT3AR882H addback also caused some remethylation at the 3b KO DMRs, suggesting that the proliferative stress of tissue culture expansion under the influence of four potent hematopoietic cytokines (IL-3, TPO, FLT3L, and SCF) increases the expression of endogenous Dnmt3a. DNMT3A protein levels are high in unmanipulated, lineage negative, WT bone marrow cells (due to progenitor enrichment, where Dnmt3a is expressed at its highest level) and high levels of DNMT3A persist for 14 days in this culture system, which contains high levels of hematopoietic growth factors that cause proliferative stress (FIG. 9).

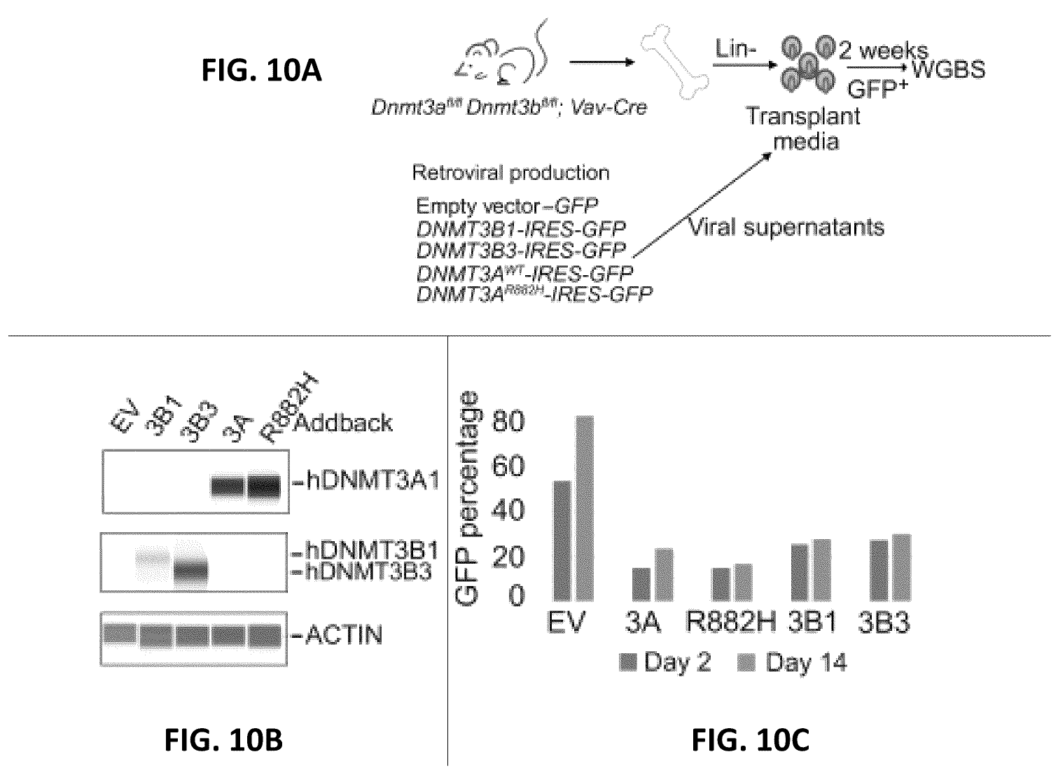

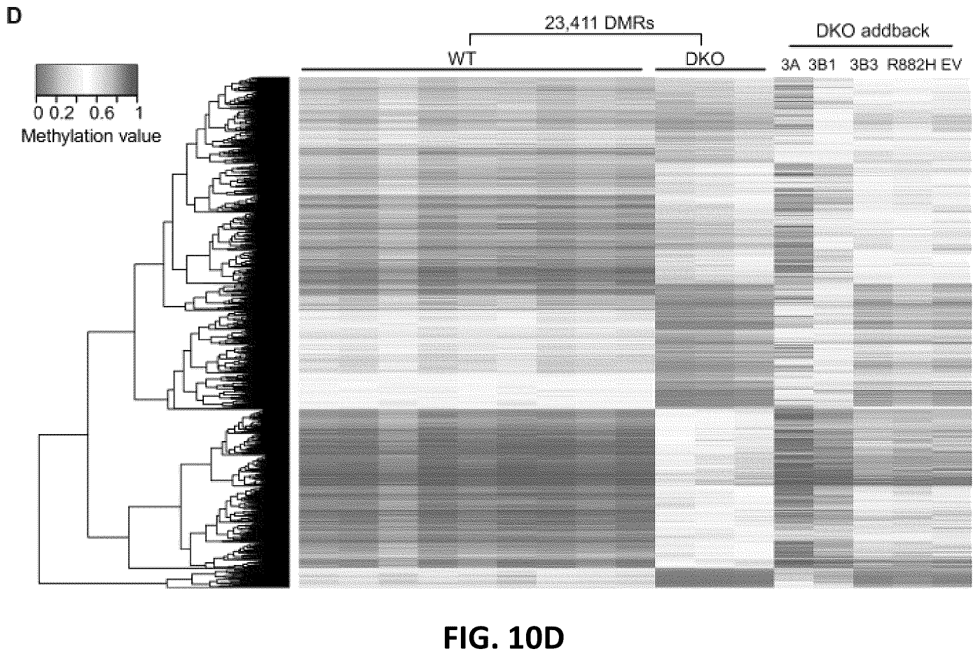

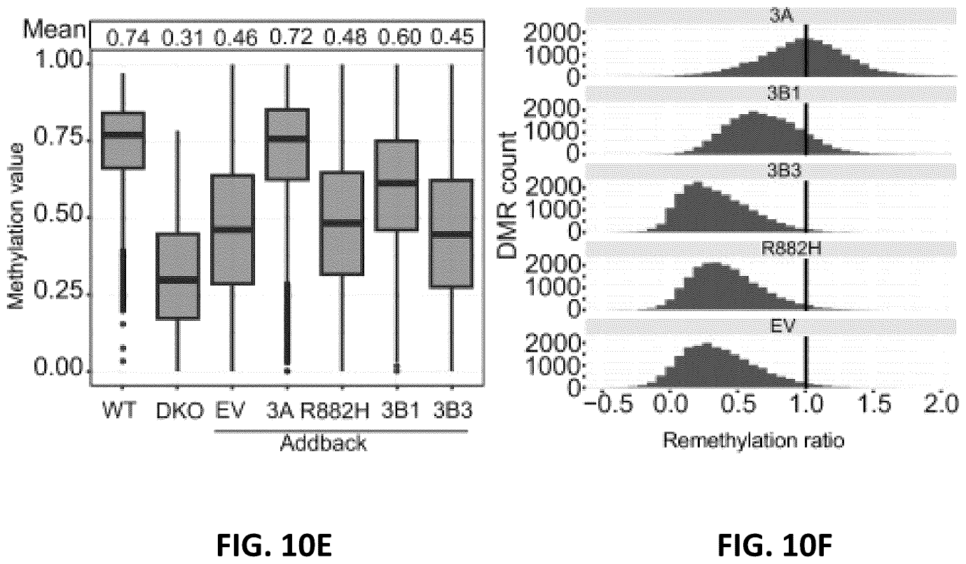

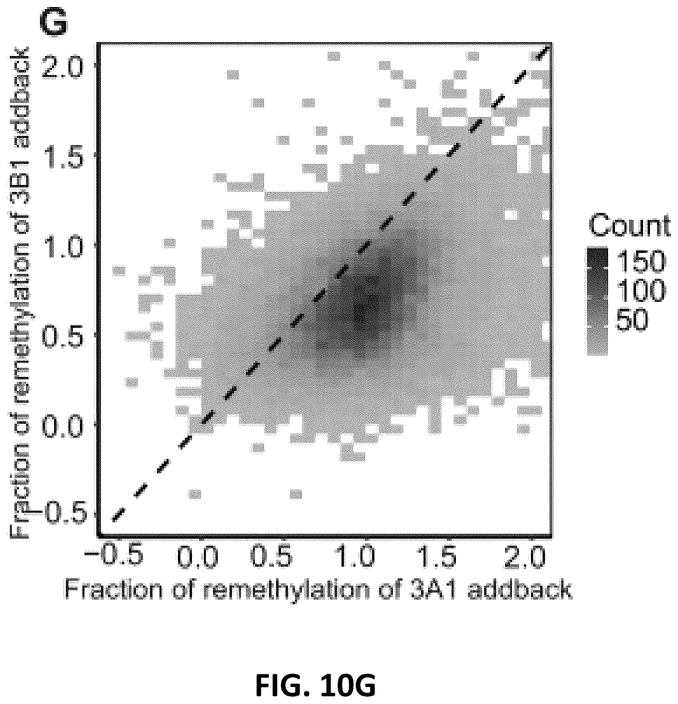

To extend these findings, the same experimental strategy was used to perform addback experiments using DKO mouse bone marrow cells (FIG. 10A). Expression of each protein in GFP+ cells on day 2 was confirmed by ProteinSimple Western assays (FIG. 10B); the fraction of GFP+ cells in each transduction was similar at days 2 and 14 (FIG. 10C). The methylation values of DKO versus WT DMRs (n=23,411) were used to calibrate the heatmap showing in FIG. 10D, and methylation values for the addback samples were plotted passively. DNMT3A1 addback restored methylation values at these DMRs to near WT levels (FIGS. 10E and 10F) was defined. DNMT3B1 addback partially remethylated these sites, but not as efficiently as DNMT3A1 (FIG. 10G). It was unclear whether levels of DNMT3A1 and DNMT3B1 overexpression were equivalent, likely due to antibodies with different affinities for their target proteins used to detect their expression. As expected, the EV, DNMT3B3-, and DNMT3AR882H-expressing vectors had equivalent levels of methylation at DKO DMRs. Some “remethylation” of DKO DMRs was detected, which may reflect normal DNA methylation in the small fraction of residual WT cells (i.e., non-floxed) in these samples.

In summary, the addback experiments described above show that DNMT3A is the dominant de novo DNA methyltransferase in hematopoietic cells and that DNMT3B3 primarily acts as a chaperone to augment the activity of DNMT3A, consistent with previous observations. These data also showed that DNMT3L, which is minimally expressed in both human and mouse hematopoietic cells, is also be capable of augmenting the activity of DNMT3A with addback. In the next set of experiments, the ability of overexpressed DNMT3A and DNMT3L was tested to overcome the dominant-negative effect of the heterozygous Dnmt3aR878H mutation in hematopoietic cells.

DNMT3L Potentiates DNMT3A Activity More than DNMT3B3

Both DNMT3L and DNMT3B3 can facilitate DNA methylation in the presence of DNMT3A, but the relative abilities of each to augment DNMT3A have not been defined. An experiment was designed to test this question, using recombinant proteins. human embryonic kidney (HEK) 293T cells were transiently transfected with eukaryotic expression plasmids (using a pcDNA3.1 backbone) containing His-tagged DNMT3A1, with or without DNMT3L or DNMT3B3 (both without His-tags). Two days after transfection, DNMT3A was purified using immobilized metal affinity chromatography Nickel resin columns, as previously described elsewhere. Tryptic peptides from the enriched proteins were identified with mass spectrometry, and protein abundance was defined after normalization to DNMT3A content in each sample and to protein mass (FIG. 11A). When DNMT3A1 was transfected alone, little-to-no endogenous DNMT3B or DNMT3L was copurified with the His-tagged DNMT3A. However, when DNMT3A1 was co-transfected with DNMT3B3, a large amount of DNMT3B was copurified (the ratio of DNMT3A:DNMT3B was 1.3:1); similarly, co-transfection of DNMT3A1 and DNMT3L yielded a large amount of copurified DNMT3L (the ratio of DNMT3A:DNMT3L was 1:4:1). Therefore, in this system, the accessory proteins DNMT3B3 or DNMT3L are “captured” and copurified via their interactions the His-tagged DNMT3A1. Methyltransferase activities of the purified proteins from each transfection (performed in triplicate) were determined using an in vitro methyltransferase assay(FIG. 11B), as previously described elsewhere. Equivalent amounts of immunoreactive DNMT3A (defined by quantitative Western blotting) were evaluated in each assay. The methyltransferase activity of co-purified DNMT3A and DNMT3B3 was significantly increased compared to that of DNMT3A alone (49% increase). The methyltransferase activity of co-purified of DNMT3A and DNMT3L was significantly increased as well and 5.5 times higher than that of DNMT3A alone. These data strongly indicate that DNMT3L is more active than DNMT3B3 as a DNMT3A1 chaperone in vitro. The ability of DNMT3B3 and DNMT3L to augment the activity of purified recombinant DNMT3AR882H protein was also evaluated, which is much less active than WT DNMT3A (FIG. 11B). DNMT3B3 co-expression resulted in a nonsignificant augmentation of activity, but DNMT3L co-expression increased its activity 4.2-fold, similar to that of the 5.5-fold augmentation of WT DNMT3A.

### Overexpression of DNMT3A or DNMT3L can Overcome the Hypomethylation Defect Caused by the Dominant-Negative Dnmt3aR878H/+ Mutation in Hematopoietic Cells

To perform these experiments, a human DNMT3L cDNA was used in a retroviral construct. To determine whether human DNMT3L could efficiently interact with mouse DNMT3A, MYC-tagged human DNMT3L cDNA was co-expressed with either a mouse Dnmt3a1 cDNA or a human DNMT3A1 cDNA (neither of which were MYC-tagged). Pull-down experiments with an anti-MYC antibody followed by Western blotting revealed that both mouse and human DNMT3A1 were capable of interacting with human DNMT3L (FIG. 11C). Retroviral addback experiments using an empty MSCV vector or vectors containing DNMT3A1 or DNMT3L cDNAs were transduced into bone marrow cells from of 6- to 8-week-old Dnmt3aR878H/+×Vav1-Cre mice (FIG. 12A). GFP+ cells were purified after 2 days of in vitro culture exactly as described above and assessed for overexpression of each protein by Western blotting (FIG. 12B). GFP+ cells from both vectors were minimally changed after 14 days of culture, suggesting that these proteins did not select for or against expressing cells (FIG. 12C). GFP+ cells were purified after 14 days from three biological replicates, and WGBS was performed. Methylation data for these samples were plotted passively for the DMRs defined for R878H versus WT bone marrow cells (FIG. 12D). Although some remethylation was observed with the EV addback alone (for reasons described above), near-complete remethylation was observed with either DNMT3A1 or DNMT3L overexpression (FIGS. 12E and 12F); remethylation was virtually indistinguishable with the two vectors (FIG. 12G). The identical experiment, when performed with 3a KO cells as the substrate, revealed near-complete restoration of methylation with DNMT3A1 addback but no remethylation (over background) with DNMT3L overexpression (FIG. 13(A-D)). These data demonstrate that DNMT3L has no intrinsic activity in this system in the absence of DNMT3A, and confirms that remethylation in R878H cells requires an intact WT Dnmt3a allele.

### Overexpression of DNMT3A or DNMT3L can Overcome the Hypomethylation Defect Caused by the DNMT3AR878H/+ Mutation In Vivo

To determine whether overexpression of DNMT3A1 or DNMT3L in vivo could correct the methylation phenotype of the R878H mutation, the experiment shown in FIG. 14A was performed. Bone marrow cells were transduced with these vectors exactly as described above. Two days after transduction, 1 million total cells from each transduction (20 to 50% GFP+) were transferred to sub-lethally irradiated (6 Gy) C57BL/6 mice via retroorbital injection. Bone marrow samples were harvested 1 month after transplantation, and GFP+ cells were harvested for analysis. DNMT3A and DNMT3L protein levels in GFP+ cells were evaluated by Western blotting on the ProteinSimple platform (FIG. 14B), revealing persistent expression of both proteins in GFP+ cells at this time point. Methylation levels for the R878H DMRs in the addback samples are passively plotted in FIG. 14C, revealing near-complete remethylation with either vector after 1 month (FIG. 14D, and FIGS. 15A and 15B) in four independent biological replicates. DNMT3A and DNMT3L addback minimally altered global methylation levels (FIG. 14(A-E) and FIG. 15C), but analysis of DMRs in all annotated genomic regions revealed near-complete correction of methylation values after 1 month in vivo (FIG. 14E).

To further define the functional consequences of overexpressing DNMT3A and DNMT3L in hematopoietic cells, scRNA-seq was performed on the GFP+ in vivo Dnmt3aR878H/+ addback samples 1 month after transplantation (FIG. 16A), as well as unmanipulated WT and Dnmt3aR878H/+ whole-bone marrow cells from littermate-matched, 8-week-old mice. The in vivo addback samples (EV, DNMT3A, and DNMT3L) were all subjected to ex vivo cytokine-driven expansion and retroviral transduction, and then transplanted into sub-lethally irradiated mice; 1 month later, the transduced, GFP+ cells were purified for scRNA-seq. These cells are therefore not equivalent to the unmanipulated marrow controls from WT versus Dnmt3aR878H/+ mice. Regardless, several key observations can be made from these data. First, persistent overexpression of the human DNMT3A and DNMT3L mRNAs for 1 month was confirmed in the scRNA-seq data (FIGS. 17A and 17B). Second, scRNA-seq data from unmanipulated bone marrow cells of 2 month old WT versus R878H mice revealed a small increase in bone marrow B cells with the R878H sample and a small decrease in mature monocytes and polymorphonuclear cells (PMNs; FIG. 16B); these data are similar to that of the bone marrow of mice with a germline Dnmt3aR878H/+ mutation. Third, the control (EV) in vivo addback R878H sample exhibited even more prominent skewing toward B cell progenitors and B cells and away from mature PMNs and monocytes (FIGS. 16A and 16C). The more notable lineage shifts (compared to the unmanipulated R878H marrow) may be related to the proliferative stress caused by transplantation. Fourth, the addback of DNMT3L and DNMT3A both caused a partial reversal of this abnormal lineage shift, reducing B cells and increasing the proportion of mature PMNs and monocytes in the addback samples (FIGS. 16A and 16C). Last, global patterns of gene expression were compared for genes within 1 kb of Dnmt3aR878H/+ DMRs, using the addback scRNA-seq data for PMNs, B cell progenitors, and monocytes, because these populations were abundant in all samples (FIG. 16D, 16E, 16F). The methylation levels at DMRs in the R878H samples were nearly completely corrected with DNMT3L or DNMT3A addback in vivo (FIG. 14(A-E) and FIG. 15(A-C), for global CpG remethylation). However, the expression of genes within 1 kb of the R878H DMRs were small and most were nonsignificant; there were <50 differentially expressed genes in each compartment for the comparison of WT versus Dnmt3aR878H/+ mice, and the global expression patterns of these genes were not notably altered with addback (FIG. 16D, 16E, 16F). This analysis was extended to all genes within 10 kb of DMRs, with similar results (FIG. 18(A-C)).

Last, to determine whether long-term overexpression of DNMT3A1 or DNMT3L might have adverse consequences in WT hematopoietic cells, in vivo addback experiments were performed using an EV or DNMT3A or DNMT3L vectors in WT mouse bone marrow cells, exactly as described in FIG. 14(A-E). One and 2 months after transplantation, purified GFP+ cells were subjected to WGBS. DMRs were identified by comparing EV GFP+ cells (from both the 1- and 2-month samples) with the 1-month samples from the DNMT3A and DNMT3L addback. Only 39 DMRs were identified, of which 34 were hypermethylated (FIG. 19A). Evaluation of the methylation status of these DMRs in the samples harvested at 2 months revealed similar findings at most sites. The nearest neighbor genes for these DMRs were not dysregulated in bulk RNA-seq datasets for WT versus R882H bone marrow samples, signifying that the expression of these genes was not affected by this mutation. Last, complete blood counts were performed on mice transplanted with marrows transduced with EV, DNMT3A, or DNMT3L after 1 or 2 months and noted only small changes (FIG. 19B).

### Discussion

As described herein, the DNA methylation phenotypes of unmanipulated primary bone marrow samples from mice that are deficient for one or both of the de novo DNA methyltransferases or expressing the Dnmt3aR878H/+ mutation. In all cases, highly efficient floxing was induced with Vav1-Cre. Focal, canonical hypomethylation phenotypes in similar areas of the genome were observed in all the mouse models, with different grades of severity depending on genotypes (DKO>3a KO>R878H>3b KO). Using a retroviral overexpression system, an efficient method was established to restore the methylation values of DMRs in each deficiency state in 14 days. Overexpression of active full-length DNMT3A1 remethylated DMRs in all DNA methyltransferase-deficient bone marrow cells. DNMT3B1 overexpression fully restored Dnmt3b KO DMRs and partially restored the methylation phenotype in other models. DNMT3B3 was inactive in 3a and DKO mice, but its addback was able to reverse the hypomethylation phenotype in 3b KO mice, indicating that it acts to increase the activity of DNMT3A in this setting. Because DNMT3L also is known to augment the activity of DNMT3A and DNMT3B during early embryogenesis, its ability was evaluated to augment the activity of the R878H mice; short-term overexpression of DNMT3L nearly completely corrected the hypomethylation phenotype of these cells within weeks, without causing inappropriate hypermethylation. Last, addback of DNMT3L or DNMT3A in vivo also had functional consequences for hematopoietic differentiation, partially correcting a differentiation block in terminal myeloid maturation associated with the Dnmt3aR878H mutation.

Previous studies have suggested that DNMT3B has distinct functions from DNMT3A and that it methylates different regions of the genome during embryogenesis. However, in both human and mouse hematopoietic cells, the dominant splice isoform detected is DNMT3B3, which is missing a portion of the catalytic domain and therefore inactive as a DNA methyltransferase. The hematopoietic cells of 3b KO mice had far fewer DMRs than 3a KO mice, and the level of hypomethylation was less severe. However, the locations of these DMRs overlapped almost completely with the 3a KO DMRs, suggesting that these two enzymes may synergize to provide the de novo methyltransferase activity of early hematopoietic cells. Addback experiments strongly implied that this synergy is a consequence of DNMT3B augmenting the activity of DNMT3A, at least in some embodiments by forming a functional heterodimer and/or heterotetramers with DNMT3A as previously suggested. The 3b KO DMRs may actually reflect a small decrease in the activity of DNMT3A, and is also supported by the similarity of the methylation phenotypes of 3b KO and R878H mice (see FIG. 3(A-H)).

The ability of DNMT3B3 to augment the function of DNMT3A is reminiscent of the chaperone function of DNMT3L in early embryogenesis. DNMT3L not only can substantially enhance the methyltransferase activity of DNMT3A in vitro and in tissue culture cells but also can increase the binding of DNMT3A to its DNA targets by changing the conformation of DNMT3A multimers. DNMT3L can also recognize modified histone marks (including unmethylated H3K4 and H3K36me3-enriched regions). Because DNMT3L is epigenetically silenced in adult hematopoietic cells (including AML cells), it likely does not contribute to the function of DNMT3A in this compartment. However, if reactivated genetically or pharmacologically, targeting DNMT3L reactivation can remethylate DNA via the resulting restored/increased DNMT3A activity if cells contain a residual WT DNMT3A allele.

The overexpression of DNMT3A1 or DNMT3L in Dnmt3aR878H/+ mouse bone marrow cells accurately restored the methylation landscape of these cells within weeks. Inappropriate hypermethylation was not detected at promoters or CpG islands nor at regions where DNMT3A is not normally active (low methylated regions). Hypermethylated regions were not detected genome-wide nor in any annotated regions of the genome. Furthermore, overexpression of DNMT3A1 or DNMT3L in WT bone marrow cells in vivo for 1 or 2 months caused only 39 DMRs in the entire genome and did not significantly alter the blood counts of these mice (FIG. 19(A-B)). These data suggest that additional (as yet undefined) factors may restrict the ability of DNMT3A to methylate some specific CpGs; further, an autoregulatory loop was implied by the downregulation of endogenous Dnmt3a and Dnmt3b expression with the in vivo addback of DNMT3A or DNMT3L for 1 month (see FIG. 17A). Alternatively, it is possible that hypermethylation is not tolerated and that cells with this phenotype are selected against. However, evidence for selection against DNMT3A overexpressing cells was not found in any in vitro addback experiment (based on stable levels of GFP+ cells overexpressing DNMT3A1 for 2 weeks after transduction). Because the correction of hypomethylated DMRs was complete within weeks for DNMT3A1 or DNMT3L addback, and because the maintenance methyltransferase, DNMT1, would be expected to maintain a corrected methylation phenotype, it may be possible to repair hypomethylation phenotypes with short-term bursts of excess DNMT3A activity mediated by DNMT3A or DNMT3L. If persistent DNMT3A loss-of-function (and its associated hypomethylation phenotype) is essential for the survival of fully transformed AML cells, transient reactivation of DNMT3L will allow for the remethylation of these cells, causing differentiation and/or growth arrest.

In summary, these observations provide a relevant strategy for reactivating DNMT3A function: re-expression of DNMT3L. The rapidity and accuracy of remethylation caused by short-term re-expression of DNMT3L do not appear to be deleterious in WT cells, nor in non-transformed hematopoietic cells expressing the Dnmt3aR878H mutation. It is as yet undetermined whether fully transformed AML cells with DNMT3AR882 mutations are “addicted” to their DNA hypomethylation phenotype, and whether its correction will slow the growth of these cells or change their developmental fate. However, therapies that are capable of reactivating the expression of DNMT3L in human cells [including HDAC inhibitors and/or HMAs] have already shown promise in treating AML patients.

### Materials and Methods

### Mouse Models

All mice were in the C57BL/6J background. Dnmt3a+/− and Dnmt3a+/− mice were generated by crossing Cre/loxP conditional mutant mice Dnmt3atm3.1 Enl from the Mutant Mouse Regional Resource Centers repository (MMRRC strain name: B6; 12954-Dnmt3atm3.1Enl/Mmnc) and Vav1-iCre mice [the Jackson Laboratory, B6.Cg-Commd10Tg(Vav1-icre)A2Kio/J]. Dnmt3b−/− mice were generated by crossing Cre/loxP conditional mutant mice Dnmt3btm5.1Enl from the Mutant Mouse Regional Resource Centers repository [MMRRC strain name: B6.129S4(Cg)-Dnmt3btm5.1 Enl/Mmnc] with Vav-iCre mice. Dnmt3a−/−×Dnmt3b−/− were generated with the Dnmt3atm3.1Enl, Dnmt3btm5.1 Enl, and Vav1-iCre mice. Mice with Dnmt3aR878H/+ mutation (mouse homolog of the human DNMT3AR882H mutation) in bone marrow cells were generated by crossing Cre/loxP conditional mutant mice C57BL/6-Dnmt3atml Rlvn/GrynvJ (the Jackson Laboratory, strain no. 031514) with Vav1-iCre mice. These mice were provided by O. Guryanova and R. Levine. Whenever possible, littermate controls were used for all experiments. All mouse studies were done in accordance with institutional guidelines and were approved by the Animal Studies Committee at Washington University.

### Retroviral Transductions

Retroviruses were generated by transfecting GP2-293 T cells (Takara Bio) with murine stem cell virus (MSCV)-internal ribosomal entry site-GFP retroviral plasmids having either no insertion, full-length WT DNMT3A1, DNMT3AR882H [generated by QuickChange II site directed mutagenesis (Agilent)], full-length DNMT3B1, DNMT3B3, or DNMT3L cDNAs using TranslT-LT1 (Muris Bio). The DNA sequences of all plasmids used in these studies were verified by whole plasmid sequencing performed by Plasmidsaurus. Supernatants were harvested at 24 and 48 hours after transfection by filtering through 0.45-μm filter (Corning) and added the filtered viral supernatants to six-well non-tissue-treated plates coated with Retronectin (Takara Bio; 5 μg/ml, 24 hours at 4° C.). The plates containing viral supernatants were spun at 2500 g for 90 min at 32° C. One to 2 million lineage depleted cells in bone marrow transplant media were added to each well of the six-well plate after the viral supernatants were discarded and were spun at 1000 rpm for 7 min at 32° C. Cells were scraped from the plates and transduced again with the 48-hour harvested viral supernatants.

### Bone Marrow Transplantation

Femur, tibia, pelvic, and humerus-derived bone marrow cells were harvested from mice in RPMI 1640 media (Gibco) with 15% FBS (Atlanta Biologicals). Bone marrow cells were treated with 1×ammonium chloride/KCl red blood cell lysis buffer and resuspended in transplant media [RPMI 1640 with 15% FBS, 1% penicillin-streptomycin (Gibco), and cytokines (mouse FLT3L, 50 ng/ml; mouse Kit Ligand, 100 ng/ml; mouse IL-3, 6 ng/ml; and mouse TPO, 10 ng/ml)] for ex vivo cultures.

Ly45.1 C57BL/6 recipients were purchased from Charles River Laboratories. Sublethal irradiation was performed by exposing mice to 6 Gy as a single dose. Transduced bone marrow cells were resuspended in phosphate-buffered saline (PBS) buffer at 1 million cells/100 pl. Transplantation was performed by injecting 1 million transduced bone marrow cells into the retro-orbital venous sinus. Mice were on antibiotic-supplemented water (sulfamethoxazole and trimethoprim) for 2 weeks after transplantation. Peripheral blood was obtained by piercing retro-orbital veins with heparinized capillary tubes (Thermo Fisher Scientific) after anesthesia with isoflurane.

### WGBS and Analysis

DNA was isolated with the QIAamp DNA Micro Kit (QIAGEN, 56304). Input DNA (50 to 100 ng) was bisulfite converted with the DNA Methylation Gold Kit (Zymo Research). Whole-genome bisulfite-converted sequencing libraries were generated with an Accel NGS Methyl-Seq DNA library kit (Swift Biosciences, no. 30096). Indexed sequencing was performed on Illumina NovoSeq 6000 instruments. Reads were mapped using biscuit, DMRs were called using metilene, and WGBS data workflow is as described previously elsewhere. TheAll data for WGBS studies were deposited in the Short Read Archive (SRA) under BioProject PRJNA1008414.

ProteinSimple Western blotting Western blotting was performed on the Jess ProteinSimple platform. Lysates were generated by sonicating cells (100,000 cells/μl) in 1×NuPAGE lithium dodecyl sulfate (LDS) sample buffer (Invitrogen) with the Sonifier 450 (Branson) setting Output Control to 3, 20% pulse for 20 s. Lysates were clarified at 13,000 rpm for 10 min at 4° C. Samples were blotted with rabbit anti-DNMT3A (Cell Signaling Technology, D23G1; 1:100 dilution), rabbit anti-DNMT3B (Cell Signaling Technology, D7070; 1:100 dilution), mouse anti-Beta-Actin (Novus, NB600-532S; 1:100 dilution), rabbit anti-DNMT3B (Abcam, ab2851; 1:50 dilution), rabbit anti-DNMT3L (Abcam, ab194094; 1:50 dilution), horseradish peroxidase (HRP)-conjugated anti-Rabbit (ProteinSimple), near-infrared (NIR)-conjugated anti-mouse (ProteinSimple), and HRP-conjugated anti-mouse (ProteinSimple). Protein abundance on blots was quantified by Compass for Simple Western software.

scRNA-Seq and Analysis

scRNA-seq was performed as previously described elsewhere. Libraries were generated with the 10× Genomics system Chromium Single Cell 5′ library Kit (v2) and sequenced on Illumina NovaSeq machines. scRNA-seq data were aligned using the Cell Ranger pipeline. Cells were annotated on the basis of the Haemopedia expression atlas. Cell populations were further defined by manual review on Partek Flow software, based on gene expression profiles. Differentially expressed genes were defined by the analysis of variance (ANOVA) algorithm with Partek Flow software. All RNA-seq data were deposited in the SRA (under BioProject PRJNA1008414).

### Total RNA-Seq and Identification of Transcript Isoforms

Total RNA-seq of Dnmt3aWT and Dnmt3aR878H/+ mice was performed using the Illumina TruSeq Stranded Total RNA Library Kit on deoxyribonuclease-treated RNA and sequenced on the NovaSeq 6000 platform, with 2×151-bp reads.

Dnmt3a and Dnmt3b isoforms were defined using the following approach. The Dnmt3a1 (Ensembl transcript ENSMUST00000020991) and Dnmt3a2 (Ensembl transcript ENSMUST00000172689) isoforms were differentiated using bulk RNA-seq and based on RegTools analysis [PubMed IDentifier (PMID): 36949070] of splice junction usage at exon 7 of the longer Dnmt3a1 transcript. Reads splicing from the 3′ untranslated region of the internal promotor to exon 7 supported Dnmt3a2, while reads splicing from exon 6 to exon 7 supported the full-length Dnmt3a1. Isoform usage percentage was calculated as the number of reads supporting one junction over the total number of reads in both junctions. Dnmt3b isoform usage was calculated similarly, using the count of splice junctions that skip exons 22 and 23 to identify the inactive Dnmt3b3 isoform (ENSMUST00000088976/ENSMUST00000103150) and the mean count of splice junctions that connect exons between 21 and 24 to determine the Dnmt3b1 counts (ENSMUST00000109774/ENSMUST00000081628). A Fisher's exact test on pooled counts was used to test the difference in proportions between WT and R878H mice.

### Recombinant DNMT3A 1 Production

This procedure was performed as previously described elsewhere. Six million HEK293T cells were plated on 15-cm plates in RPMI 1640 (Gibco) [including 10% FBS (Atlanta Biologicals) and 1% penicillin-streptomycin (Gibco)] overnight. Total 6×His-DNMT3A-FLAG expression plasmids (25 μg), 6×His-DNMT3A-FLAG (12.5 μg) with 12.5 μg of DNMT3B3-myc expression plasmids, or 6×His-DNMT3A-FLAG (12.5 μg) with 12.5 μg of DNMT3L-myc expression plasmids were transfected with calcium-phosphate transfection methods on the following day. Medium was changed without disturbing the cells 16 hours after transfection. Cells were harvested in cold PBS at 48 hours after transfection and resuspended at 10 million cells/ml in lysis buffer [20 mM sodium phosphate (pH 7.65), 150 mM NaCl, and 20 mM imidazole]. Cell lysates were prepared by sonication, using a Sonifier 450 (Branson) (8 s, Output Control to 3.5, constant, repeated three times). Lysates were clarified at 13,000 rpm for 10 min at 4° C. The supernatants were passed through 0.45-μm filters (Corning) and loaded into a 1-ml HisTrap HP column (Cytiva) and washed with 10 ml of buffer [20 mM sodium phosphate (pH 7.65), 150 mM NaCl, and 50 mM imidazole]. Proteins were eluted in elution buffer [20 mM sodium phosphate (pH 7.65), 150 mM NaCl, and 400 mM imidazole] and dialyzed into 20 mM Hepes (pH 7.65), 30 mM NaCl, and 1 mM EDTA with 10% glycerol. DNMT3A protein abundance was measured using Western blotting using the ProteinSimple Jess system. Purified proteins were stored at −80° C. in individual aliquots and never subjected to more than one freeze-thaw cycle.

Mass spectrometry and data analysis Purified samples with recombinant DNMT3A were digested for 2 hours at 30° C. in a ThermoMixer with gyration at 750 rpm. Trypsin (1 μg) was added, and the samples were incubated overnight at 30° C. in the ThermoMixer, gyrating at 750 rpm. Tryptic peptides were then transferred to a fresh tube, the bead samples were washed with an additional 50 μl of ammonium bicarbonate (ABC) buffer, and the wash was combined with the peptides. Residual detergent was removed by ethyl acetate extraction. In preparation for desalting, peptides were acidified to pH 2 with 1% trifluoroacetic acid (TFA) final concentration. The peptides were desalted using two micro-tips (porous graphite carbon, BIOMETNT3CAR) (Glygen) on a Beckman robot (Biomek NX). The peptides were eluted with 60% MeCN in 0.1% TFA and dried in a Speed-Vac (Thermo Scientific, Model No. Savant DNA 120 concentrator) after adding TFA to 5%. The peptides were dissolved in 20 μl of 1% MeCN in water. An aliquot (10%) was removed for quantification using the Pierce Quantitative Fluorometric Peptide Assay kit (Thermo Fisher Scientific, cat. no. 23290). The remaining peptides were transferred to autosampler vials (Sun-Sri, cat. no. 200046), dried, and stored at −80° C. for liquid chromatography-mass spectrometry (MS) analysis on timsTOF Pro mass spectrometer. Peptide counts were normalized according to the size of each protein, and relative abundance was calculated after normalization to detected levels of DNMT3A as follows: Data from the mass spectrometer were converted to peak lists using Proteome Discoverer (version 2.1.0.81, Thermo Fisher Scientific). The MS2 spectra with charges+2, +3, and +4 were analyzed using Mascot software (Matrix Science, London, UK; version 2.8.1). The searches were performed with a mass tolerance of 20 parts per million for both precursor and fragment ions. Tandem MS spectra were set up to search against a UniProt database of human proteins (January 2023; 20,423 entries) and common contaminant proteins (version 1.0; January 2012; 116 entries), assuming that the digestion enzyme was trypsin/P with a maximum of four missed cleavages allowed. Carbamidomethylation of cysteine was specified as a fixed modification. Deamidation of asparagine and glutamine, formation of pyro-glutamic acid from N-terminal glutamine, acetylation of protein N terminus, and oxidation of methionine were specified as variable modifications. Peptide spectrum matches (PSMs) were filtered at 1% FDR by searching against a reversed database and the ascribed peptide, and protein identities were accepted.

The precursor intensities were converted to logarithmic ratios at base two (log 2FC), relative to the average precursor intensity across all samples. Under each sample, Dixon's outlier removals were carried out recursively for peptides with greater than two identifying PSMs. The median of the PSM log 2FC that could be assigned to the same peptide was taken to represent the ratios of the incumbent peptide. The median of the peptide log 2FC was taken to represent the log 2FC of the inferred protein. To align protein ratios across samples, likelihood functions were first estimated for the protein log 2FC using finite mixture modeling (R package: mixtools::normalmixEM), assuming two-component Gaussian mixtures. The distributions of log 2FC were then aligned so that maximum likelihood was centered at zero for each sample. Scaling normalization was performed to standardize the protein log 2FC across all samples. To reduce the influence of outliers, the values between the 5th and 95th percentile of log 2FC and 5th and 95th percentile of intensity were used in the calculations of SDs. Following the normalization of protein log 2FC, protein intensities were calibrated by an anti-logarithmic conversion of 2d, where d is the distance of log 2FC before and after the ratio normalization. The obtained protein intensities were used for protein stoichiometry predictions. These data are available at ProteomeXchange (accession number: PXD044808).

In vitro methyltransferase assay Equivalent amount of immunoreactive DNMT3A, defined by quantification with Western blotting (DNMT3A, DNMT3A/DNMT3B3, and DNMT3A/DNMT3L), was incubated with 5 μM 3H-labeled S-Adenosyl methionine (SAM) (PerkinElmer) and 1 μg of pcDNA3.1 (Invitrogen) in 20 mM Hepes (pH 7.65), 30 mM NaCl, 1 mM EDTA, 0.5 mM dithiothreitol, and bovine serum albumin (0.2 mg/ml) at 37° C. for 20 hours. Samples were spotted on filters in NucleoSpin Gel and PCR clean-up kits (Macherey-Nagel) and washed twice with the washing buffer provided in the kit. The columns were dried by centrifugation at 13,000 rpm for 2 min, and incorporated radioactivity was measured by liquid scintillation (Beckman Coulter LS 6500).

### Coimmunoprecipitation Assays

Three million K562 cells were electroporated with 4 μg of a plasmid expressing a human DNMT3L-myc-tagged cDNA alone or with 4 μg of plasmids expressing either a human DNMT3A cDNA or a mouse Dnmt3a cDNA, using the Lonza SF cell line kit (Lonza, V4XC-2024). After electroporation, cells were cultured in RPMI 1640 media with 10% FBS and harvested 24 later in 500 μl of Pierce IP lysis buffer (Thermo Fisher Scientific, product no. 87787), followed by sonication with Sonifier 450 (Branson) setting Output Control to 3, 30% pulse, for 20 s. Lysates were clarified at 13,000 rpm for 10 min at 4° C. Myc-binding beads were prepared by adding 10 μl of a conjugated anti-myc-tag antibody (Cell Signaling Technology, no. 9B11) with 50 μl of prewashed Pierce Protein A/G Magnetic Beads (Thermo Fisher Scientific, product no. 88802) in 250 μl of Pierce IP lysis buffer for 1 hour at 4° C. Supernatants were removed, and then 500 μl of the clarified cell lysates were added to each tube, rotating the mixture gently overnight at 4° C. Beads were purified using a magnetic stand and washed three times with 500 μl of 1×tris-buffered saline (Thermo Fisher Scientific, product no. 28360). Last, 100 μl of LDS sample buffer was added to the beads, and proteins were eluted at 99° C. for 10 min; the supernatants were then analyzed by Western blotting on the ProteinSimple platform. Antibodies used for Western blotting included anti-myc-tag antibody (Cell Signaling Technology, no. 9B11) for Myc-tagged DNMT3L and anti-DNMT3A antibody (Cell Signaling Technology, no. D23G1) for DNMT3A, which cross-reacts with mouse and human DNMT3A.

### Statistical Comparisons

All statistical comparisons were made using GraphPad Prism 5 software, except for statistics on sequencing data, which were calculated using the R statistical programming software as described above. Statistical tests used and significance cutoffs are detailed in each figure legend. All data represent means±SD or SEM, as specified in the figure legends.

What is claimed is:

1. A method of increasing DNMT3A activity in a subject having a DNMT3A mutation, the method comprising:
reactivating expression of DNMT3L by administering a retroviral vector comprising DNMT3L cDNA to the subject.

2. The method of claim 1, wherein the DNMT3A mutation is selected from a loss-of function mutation and a dominant negative mutation.

3. The method of claim 2, wherein the DNMT3A mutation is selected from R882H and R878H.

4. The method of claim 1, wherein the subject has a DNMT3A deficiency-associated disease selected from leukemia, acute myeloid leukemia (AML), a lymphoid malignancy, clonal hematopoiesis, and DNMT3A Overgrowth Syndrome.

5. The method of claim 1, further comprising administering a therapeutic agent selected from an histone deacetylase (HDAC) inhibitor and a hypomethylating agent.

6. The method of claim 5, wherein the therapeutic agent is selected from Azacitidine, Romidepsin, and 5-azacytidine.

7. A method of reversing a hypomethylation phenotype in bone marrow cells of a subject having a DNMT3A mutation, the method comprising:
increasing DNMT3A activity by administering a retroviral vector comprising one or cDNA vectors selected from DNMT3L, a combination of DNMT3A and DNMT3L, and a combination of DNMT3A and DNMT3B.

8. The method of claim 7, wherein the retroviral vector comprising is administered directly to the bone marrow cells of the subject.

9. The method of claim 7, wherein the DNMT3A mutation is selected from a loss-of function mutation and a dominant negative mutation.

10. The method of claim 9, wherein the DNMT3A mutation is selected from R882H and R878H.

11. The method of claim 7, wherein the subject has a DNMT3A deficiency-associated disease selected from leukemia, acute myeloid leukemia (AML), a lymphoid malignancy, clonal hematopoiesis, and DNMT3A Overgrowth Syndrome.

12. The method of claim 7, further comprising administering a therapeutic agent selected from an histone deacetylase (HDAC) inhibitor and a hypomethylating agent.

13. The method of claim 12, wherein the therapeutic agent is selected from Azacitidine, Romidepsin, and 5-azacytidine.

14. A method of promoting cancer cell death in a subject having Acute Myeloid Leukemia (AML), the method comprising:
increasing DNMT3A activity by administering a retroviral vector comprising one or cDNA vectors selected from DNMT3L, a combination of DNMT3A and DNMT3L, and a combination of DNMT3A and DNMT3B.

15. The method of claim 14, wherein the retroviral vector is administered directly to bone marrow cells of the subject.

16. The method of claim 14, wherein the subject has a loss-of function DNMT3A mutation.

17. The method of claim 14, wherein the subject has a dominant negative DNMT3A mutation.

18. The method of claim 14, wherein the subject has a DNMT3A mutation selected from R882H and R878H.

19. The method of claim 14, further comprising administering a therapeutic agent selected from an histone deacetylase (HDAC) inhibitor and a hypomethylating agent.

20. The method of claim 19, wherein the therapeutic agent is selected from Azacitidine, Romidepsin, and 5-azacytidine.
